(19) United States
(12) Patent Application Publication
(10) Pub. No.: US 2024/0084391 A1
(43) Pub. Date: Mar. 14, 2024

(54) Diagnostic Methods and Methods of Treatment of Ovarian Cancer

(71) Applicant: Genentech, Inc., South San Francisco, CA (US)

(72) Inventors: Milena Rosa Hornburg, Foster City, CA (US); Yulei Wang, Foster City, CA (US)

(73) Assignee: Genentech, Inc., South San Francisco, CA (US)

(21) Appl. No.: 18/237,959

(22) Filed: Aug. 25, 2023

Related U.S. Application Data
(63) Continuation of application No. PCT/US22/17890, filed on Feb. 25, 2022.
(60) Provisional application No. 63/155,089, filed on Mar. 1, 2021.
(60) Provisional application No. 63/222,167, filed on Jul. 15, 2021.
(60) Provisional application No. 63/155,089, filed on Mar. 1, 2021.

(51) Int. Cl. C12Q 1/6886; G01N 33/574

(52) U.S. Cl. CPC C12Q 1/6886; G01N 33/57449; C12Q 2600/158; G01N 2800/52

(57) ABSTRACT

This application discloses methods of designing a treatment protocol for a human patient with ovarian cancer, as well as methods of treatment of ovarian cancer. This application also includes methods of characterizing an ovarian cancer in a human patient by the type of tumor.

### CROSS-REFERENCE TO RELATED APPLICATIONS

This application is a continuation of International Application No. PCT/US2022/017890, filed Feb. 25, 2022, which claims the benefits of priority of US Provisional Application Nos. 63/155,089, filed Mar. 1, 2021, and 63/222,167, filed Jul. 15, 2021, both of which are incorporated by reference herein in their entireties.

### FIELD

Methods of designing a treatment protocol for a human patient with ovarian cancer, methods of treatment of ovarian cancer, and methods of characterizing an ovarian cancer in a human patient by the type of tumor.

### BACKGROUND

The tumor microenvironment (TME) is a complex ecosystem comprised of tumor cells, infiltrating immune cells, and stromal cells intertwined with non-cellular components. The diverse cellular and functional phenotypes, as well as the dynamic interplay within and between these components, may shape a tumor's distinct biology and contribute to different responses to immunotherapies. However, a high-resolution characterization of these important cellular heterogeneities and interactions is lacking. Most of the previous studies relied on relatively low-resolution techniques such as immunohistochemistry (IHC) or bulk RNA sequencing (RNAseq) deconvolution algorithms (e.g., CIBERSORT, xCell). Zhang, L. et al., N. Engl. J. Med., 348:2023-213 (2003); Newman, A. et al., Nat. Meth., 12:453-457 (2015); Aran, D. et al., Genome Biol., 18:202, doi:10.1186/s13059-017-1349-1 (2017). Although recent work integrated multi-omics platforms and in situ lymphocyte quantifications identifying distinct immune phenotypes (Thorsson, V. et al., Immunity, 48:812-830.e14 (2018)), these studies lacked higher-resolution information on cellular heterogeneity and spatial distribution.

The concept of a tumor immunity continuum was introduced to better capture the spatial distribution of the immune infiltrates in addition to overall quantities. Hegde, P. S. et al., Clinical Cancer Research, 22:1865-1874 (2016); Hegde, P. S. Chen, D. S., Immunity, 52:17-35 (2020). The TME continuum comprises three immune phenotypes based on the spatial distribution of T cells in the TME: (1) the immune inflamed/infiltrated phenotype where the T cells infiltrate the tumor epithelium; (2) the immune excluded phenotype in which infiltrating T cells accumulate in the tumor stroma rather than the tumor epithelium, and (3) the immune desert phenotype in which T cells are either present in very low numbers or completely absent. Building upon this model, a machine learning approach was developed that integrates digital pathology CD8 IHC with bulk transcriptome analysis to classify ovarian tumors according to their tumor immune phenotypes. Desbois, M. et al., Cancer Res., 79:463 (2019). This classification enabled characterization of features associated with the different immune phenotypes based on bulk RNAseq data. However, an innate limitation of bulk RNAseq is that it lacks (1) the resolution to interrogate the heterogeneity of TME at the cellular level; and (2) the sensitivity to capture changes in underrepresented cell populations. To this end, single-cell RNA sequencing (scRNAseq) has been used over the last decade to dissect the composition of the TME in various cancer indications. Guo, X. et al., Nature Medicine, 24:978-985 (2018); Lambrechts, D. et al., Nature Medicine, 24:1277-1289 (2018); Li, H. et al., Cell, 176:77-789, doi:10.1016/j.cell.2018.11.043 (2019); Puram, S. V. et al., Cell, 171:1611-1624.e24 (2017); Savas, P. et al., Nature Medicine, 24:986-993 (2018); Tirosh, I. et al., Science, 352:189-196 (2016); Wu, T. D. et al., Nature, 579:274-278 (2020); Yost, K. E. et al., Nature Medicine, 25:1251-1259 (2019); Zhang, L. et al., Nature, 54:321-33 (2018); Zhang, Q. et al., Cell 179:829-845 (2019). However, most scRNAseq studies focused on the characterization of tumor infiltrating T cells. A systematic single-cell characterization of how other cell types in the TME shape the immune phenotypes has not been reported to date.

### SUMMARY

The present disclosure relates to diagnostic methods, methods of treatment, and methods for predicting patient outcomes for human ovarian cancer. The disclosure includes multiple embodiments, including, but not limited to, the following embodiments.

Embodiment 1 is a method of designing a treatment protocol for a human patient with ovarian cancer comprising:
a. determining the expression level of at least one of GZMK, TREM1, and TREM2 in a tumor sample from the patient; and
b. comparing the expression level to the expression level in a reference sample, wherein an increased level of GZMK expression compared to the reference sample is associated with reduced survival, an increased level of TREM1 compared to the reference sample is associated with the presence of MDSC-like myeloid cells, and an increased level of TREM2 compared to the reference sample is associated with the presence of TAM-like macrophages.

Embodiment 2 is a method treatment of ovarian cancer in a human patient comprising:
a. determining the expression level of at least one of GZMK, TREM1, and TREM2 in a tumor sample from the patient,
b. comparing the expression level to the expression level in a reference sample, wherein an increased level of GZMK expression is associated with reduced survival, an increased level of TREM1 is associated with the presence of MDSC-like myeloid cells, and an increased level of TREM2 is associated with the presence of TAM-like macrophages; and
c. if the patient has
   i. increased expression of TREM1 compared to the reference sample, administering to the patient a therapy targeting MDSC-like myeloid cells;
   ii. increased expression of TREM2 compared to the reference sample, administering to the patient a therapy targeting TAM-like macrophages; and/or
   iii. decreased expression of GZMK compared to the reference sample, administering to the patient chemotherapy.

Embodiment 3 is the method of treatment of embodiment 1 or 2, wherein after increased GZMK expression has been shown in the tumor, chemotherapy is stopped. In some embodiments, the GZMK level is increased relative to the same expression level at an earlier time point while on chemotherapy (in the same patient). In some embodiments, the GZMK level is increased relative to a reference sample.

Embodiment 4 is the method of treatment of any one of embodiments 1-3, wherein after increased GZMK expression has been shown in the tumor, palliative care is given to the patient.

Embodiment 5 is the method of characterizing an ovarian cancer in a human patient as a desert, excluded, or infiltrated type of tumor comprising:
a. determining the expression level of at least one of GZMB, GZMK, CD8A, TREM1, and TREM2 in a tumor sample from the patient,
b. comparing the expression level to the expression level in a reference sample, wherein higher expression of GZMB compared to the reference sample is associated with infiltrated type of tumors, higher expression of GZMK compared to the reference sample is associated with excluded tumors, higher expression of CD8A/GZMK double positive cells compared to the reference sample is associated with excluded tumors, higher expression of TREM1 compared to the reference sample is associated with desert tumors, and higher expression of TREM2 compared to the reference sample is associated with excluded and infiltrated tumors.

Embodiment 6 is the method of treating a human patient with ovarian cancer comprising:
a. characterizing an ovarian cancer as desert, excluded, or infiltrated by determining the expression level of at least one of GZMB, GZMK, CD8A, TREM1, and TREM2 in a tumor sample from the patient,
b. comparing the expression level to the expression level in a reference sample, wherein higher expression of GZMB compared to the reference sample is associated with infiltrated type of tumors, higher expression of GZMK compared to the reference sample is associated with excluded tumors, higher expression of CD8A/GZMK double positive cells compared to the reference sample is associated with excluded tumors, higher expression of TREM1 compared to the reference sample is associated with desert tumors, and higher expression of TREM2 compared to the reference sample is associated with excluded and infiltrated tumors and
c. treating the patient with:
   i. chemotherapy or autologous/allogenic effector cells if the higher expression of TREM1 suggests a desert tumor;
   ii. immunotherapy (including checkpoint therapy) if the higher expression of GZMB and/or TREM2 suggests an infiltrated tumor;
   iii. palliative care if the higher expression of GZMK suggests an excluded tumor;
   iv. palliative care if the higher expression of CD8A/GZMK double positive cells suggests an excluded tumor; and/or
   v. immune effector cells, such as T cell trafficking modulators, epigenetic modulators, tumor microenvironment (TME) remodeling molecules, and/or radiation therapy.

Embodiment 7 is the method of any one of embodiments 1-6, where the reference sample is a healthy subject.

Embodiment 8 is the method of any one of embodiments 1-6, wherein the reference sample is a patient who responded to therapy, and in some cases the patient may be a patient with ovarian cancer.

Embodiment 9 is the method of any one of embodiments 1-6, wherein the reference sample is a patient who has a known desert tumor, and in some cases the patient may be a patient with ovarian cancer.

Embodiment 10 is the method of any one of embodiments 1-6, wherein the reference sample is a patient who has a known excluded tumor, and in some cases the patient may be a patient with ovarian cancer.

Embodiment 11 is the method of any one of embodiments 1-6, wherein the reference sample is a patient who has a known infiltrated tumor, and in some cases the patient may be a patient with ovarian cancer.

Embodiment 12 is the method of any one of embodiments 1-6, wherein the reference sample is data compiled across a plurality of patients and/or subjects, and in some cases the patient may be a patient with ovarian cancer.

Embodiment 13 is the method of any one of embodiments 5-12, wherein the method comprises evaluating all of GMZB, TREM1, and TREM2 in a tumor cell from the patient. The tumor cell may be a stromal or immune cell from the tumor.

Embodiment 14 is the method of any one of embodiments 1-4 or 7-13, wherein the method comprises evaluating all of GMZK, TREM1, and TREM2 in a tumor cell from the patient.

Embodiment 15 is the method of any one of embodiments 1-14, wherein the method further comprises obtaining a tumor sample from the patient before determining the expression level of at least one of GZMB, GZMK, TREM1, and TREM2.

Embodiment 16 is the method of any one of embodiments 1-15, wherein the expression level of GZMB, GZMK, TREM1, and/or TREM2 is determined using immunohistochemistry.

Embodiment 17 is the method of any one of embodiments 1-15, wherein the expression level of GZMB, GZMK, TREM1, and/or TREM2 is determined by measuring mRNA transcript levels.

Embodiment 18 is the method of embodiments 17, wherein the method further comprises determining the expression level of at least one reference gene in the tumor sample, i.e., wherein the reference gene is the same gene as the gene for which the investigator is determining the expression level of at least one of in a tumor sample from the patient.

Embodiment 19 is the method of embodiments 17 or 18, wherein the method further comprises normalizing the level of the mRNA transcripts against a level of an mRNA transcript of the at least one reference gene in the tumor sample to provide a normalized level of the mRNA transcript of GZMB, GZMK, TREM1, and/or TREM2.

Embodiment 20 is the method of any one of embodiments 17-19, wherein the levels of the mRNA transcripts is determined by scRNAseq.

Embodiment 21 is the method of any one of embodiments 1-20, wherein the tumor sample is separated into tumor, stromal, and immune cells before evaluating the expression level of at least one of GZMB, GZMK, TREM1, and or TREM2.

Embodiment 22 is the method of embodiments 21, wherein the cell separation occurs through FACS.

Embodiment 23 is the method of any one of embodiments 19-22, wherein an increased normalized level of mRNA transcripts of GZMK is in CD8+ T cells.

Embodiment 24 is the method of embodiments 23, wherein the number of CD8+ T cells that are GZMK positive are greater than the number of CD8+ T cells that are GZMK negative.

Embodiment 25 is the method of any one of embodiments 19-24, wherein the increased normalized level of mRNA transcripts of TREM2 is in macrophages.

Embodiment 26 is the method of any one of embodiments 2-4 or 6-25, wherein the chemotherapy comprises Albumin bound paclitaxel (nab-paclitaxel (Abraxane®)), altretamine (Hexalen®), Capecitabine (Xeloda®), carboplatin, cisplatin, cyclophosphamide (Cytoxan®), docetaxel (Taxotere®), etoposide (VP-16), gemcitabine (Gemzar®), ifosfamide (Ifex®), irinotecan (CPT-11 (Camptosar®)), doxorubicin (such as liposomal doxorubicin (Doxil®)), melphalan, paclitaxel (Taxol®), pemetrexed (Alimta®), topotecan, vinorelbine (Navelbine®), Niclosamide, Metformin, BAY 87-2243, Decitabine, Guadecitabine, Azacytidine, Abagovomab, Oregovomab, NeoVax with Nivolumab, Anlotinib, Enoxaparin with Rosuvastatin, Niraparib, Chiauranib, Trabectedin with pegylated liposomal Doxorubicin, ACB-S6-500, SGI-110, Letrozole, Pazopanib, Palbociclib, Apatinib, Masitinib, Cabazitaxel, IMAB027, Fludarabine, ABT-888, Fostamatinib, Olaparib, Temozolomide, Talazoparib, P53-SLP, OMP-54F28, Hydralazine and magnesium valproate, Fludarabine, Lapatinib, Bendamustine HCL, Sorafenib, Camrelizumab, Tremelilumab, Tocotrienol, and/or Exemestane.

Embodiment 27 is the method of any one of embodiments 2-4, 7-12, or 14-25, wherein a therapy targeting MDSC myeloid cells comprises:
a. Cisplatin, 5-flurouracil, gemcitabine, and/or paclitaxel;
b. Liver X receptor (LXR) beta agonist;
c. a checkpoint inhibitor, such as
   i. anti-PD-1 therapy (such as anti-PD-1 antibodies including pembrolizumab (Ketruda®), nivolumab (Opdivo®), cemiplimab (Libtayo®), Toripalimab, CT-011, monoclonal antibody HX0088, and antibody AK105;
   ii. anti-PD-L1 therapy (such as anti PD-L1 antibodies including atezolizumab (Tecentriq®), avelumab (Bavencio®), durvalumab (MEDI4736 anti-PD-L1; Imfinzi®), Tremelimumab, mAb ZKAB001, Tremelimumab, and Ramucirumab (Cyramza); and/or
   d. Anti-TGFβ therapy (such as anti-TGFβ antibodies including Pembrolizumab and Fresolimumab).

Embodiment 28 is the method of any one of embodiments 6-13 or 15-26, wherein the cancer immunotherapy agent comprises Durvalumab (MEDI4736 anti-PD-L1; Imfinzi®), motolimod, oncolytic virus, NY-ESO-1 cancer vaccine, anti-XBP1 therapy, anti-angiopoietin therapy, anti-DLL/Notch therapy, anti-HER2 therapy, anti-mesothelin therapy, anti-RANKL therapy, anti-TROP2 therapy, and/or VEGF/VEGF-R therapy.

### BRIEF DESCRIPTION OF THE DRAWINGS

FIGS. 1A-G shows tumor, immune, and stromal compartments from all 15 ovarian cancer samples analyzed by scRNAseq. FIG. 1A shows a uMAP projection of all cells aggregated from all sequenced libraries, combining tumor, stromal and immune compartments of all patients. Cells are colored according to their FACS sorted compartments. Stromal and immune cells that have been combined for the single cell RNA sequencing are colored in dark blue. FIG. 1B shows a uMAP indexed by seurat louvain clustering. Cluster 34 was identified as normal epithelial cells based on the expression of epithelial markers (PIFO, CAPS, TMEM190, SNTN) and was removed from further analysis. Lambretchs, D. et al., Nat. Med. 1-19, doi:10.1038/s41591-018-0096-5 (2018); Wu, T. D. et al., Nature, 579:274-278 (2020). FIG. 1C shows a uMAP projection as in B, but with normal epithelial cells removed, and colored by patient identity. FIG. 1D shows a uMAP projection of computationally filtered subset of cells and colored by computationally assigned tumor, stromal and immune compartments. FIG. 1E shows marker gene expression of representative stromal cell markers (COL3A1, DCN), immune cell markers (PTPRC, CD79A) and normal epithelial cell markers (PIFO, CAPS, TMEM190, SNTN). Darker shades indicate higher expression. FIG. 1F shows a uMAP projection of stromal cells of all patients colored by the patient origin. FIG. 1G shows a uMAP projection of immune cells of all patients colored by the patient origin.

FIGS. 2A-F show the complete cellular ecosystem of patient-derived primary ovarian tumors as defined by scRNAseq. FIG. 2A shows an overview of the study design and workflow. Bulk RNA sequencing and CD8 IHC staining was performed for 42 ovarian cancer samples. The combined information was used to determine the immune phenotype of each sample. Of the 42 samples, 15 samples with the most clear immune phenotype were selected, 5 of each immune phenotype: infiltrated, excluded and desert. From each single cell dissociated sample, the tumor, immune and stromal cell populations were sorted and then subjected to 10× single cell RNA sequencing. Computational analyses included cluster, celltype and single patient analysis, batch effect correction and functional as well as cell interaction analysis. Findings were validated using immunofluorescence and RNAish assays, and deconvolution analyses of clinical bulk RNAseq datasets. FIG. 2B shows a uMAP projection of tumor cells of all 15 patients colored by patient. Abbreviations in the plot legend correspond to the tumor immune phenotype and the patient number within each tumor immune phenotype: des=desert, exc=excluded, inf=infiltrated. FIG. 2C shows a uMAP projection of stromal cells of all patients colored by the identified cell type. Identical uMAPs show the cell type marker gene expression levels. Darker shades indicate higher expression. FIG. 2D shows a uMAP projection of immune cells of all patients colored by the identified cell types. Identical uMAPs show the cell type marker gene expression levels. Darker shades indicate higher expression. FIG. 2E shows stromal cell type fractions compared to total stromal cell count per patient. Each stacked bar represents a patient for which the total stromal cell count was scaled to 1. FIG. 2F shows immune cell type fractions compared to the total immune cell count per patient.

FIGS. 3A-D show gene expression and immune cell infiltration analyses of scRNAseq selected ovarian cancer samples. FIG. 3A shows a heatmap of immune phenotype classifier genes, previously described in Desbois, M et al., Nature Communications, 11:5583, doi:10.1038/s41467-020-19408-2 (2020). Gene expression from bulk RNA sequencing of 15 ovarian cancer samples. FIG. 3B shows CD8 IHC staining of the 15 selected ovarian cancer samples: 5 infiltrated, 5 excluded and 5 desert tumors. FIG. 3C shows flow cytometry gating strategy for the cell sorting of the tumor, stromal and immune cells of each patient. FIG. 3D shows percentage of FACS sorted EpCAM+ (tumor), CD45+(immune) and CD45− EpCAM− (stromal) cells for each patient and grouped by immune phenotype. The graphs show the mean±SD where each dot represents a patient. Stacked boxplot shows the compartment distribution by immune phenotype using the mean percentage of tumor, stromal, immune cells among viable cells.

FIGS. 4A-C show tumor-intrinsic features associated with different tumor immune phenotypes. FIG. 4A shows significantly enriched HALLMARK gene sets in the tumor cells of desert compared to the combined set of excluded/infiltrated tumors (adjusted p-value<0.05). Pseudobulk differential expression analysis was performed comparing the tumor cell expression of desert tumors to excluded/infiltrated tumors while correcting for the G2/M status of each tumor in the differential expression model (Methods). Fold change expression values and adjusted p-values were combined to rank the genes as input for the gene set enrichment analysis. FIG. 4B shows a heatmap of the antigen presentation gene set expression by patient. Z-scored gene expression was calculated based on scran normalized expression values. Violin plot shows the distribution of the antigen presentation signature score per cell by immune phenotype. The antigen presentation signature score was calculated based on the genes shown in the heatmap. FIG. 4C shows a heatmap of oxidative phosphorylation gene set expression by patient. Z-scored gene expression was calculated based on scran normalized expression values. Violin plot shows the distribution of the oxidative phosphorylation signature score per cell by immune phenotype. The signature score was calculated based on the genes shown in the heatmap.

FIGS. 5A-B show tumor, immune, and stromal compartments from all 15 ovarian cancer samples analyzed by scRNAseq. FIG. 5A shows a heatmap of the Hallmark EMT leading edge genes from desert (des) vs infiltrated (inf)/excluded (exc) gene set enrichment analyses. FIG. 5B shows a heatmap of the Hallmark angiogenesis leading edge genes from des vs inf/exc gene set enrichment analyses.

FIG. 6 shows cellular and functional characterization of T cells infiltrating ovarian tumors. FIG. 6 shows a uMAP project of T cells of all patients before and after batch balanced k-nearest neighbor correction.

FIG. 7 shows characterization of myeloid cell subsets in ovarian cancer. FIG. 7 shows a uMAP projection of myeloid cells of all patients before and after batch balanced k-nearest neighbor correction.

FIGS. 8A-J show that distinct states of CD8+ T cells characterize immune infiltrated and excluded tumors immune phenotypes. FIG. 8A shows a uMAP projection of all T cells from all patients colored by identified cell populations. FIG. 8B shows a uMAP projection of all T cells colored by the expression of CD4+ and CD8+ T cell markers. Darker shades indicate higher expression. FIG. 8C shows a heatmap of z-scored gene expression of the top 20 significant markers (adjusted p-value<0.05) of each T cell population, selected gene labels are shown. Z-scored gene expression was calculated based on scran normalized and per cell population averaged expression values. FIG. 8D shows a uMAP projection of all T cells colored by the expression of GZMB and GMZK. Darker shades indicate higher expression. FIG. 8E shows a heatmap of the z-scored gene expression values of T cell activation and exhaustion markers for the CD8+ GZMB and CD8+ GZMK populations. The z-scores were calculated based on the average scran normalized expression of all cells in the respective cell population and from the excluded or infiltrated immune phenotype. FIG. 8F shows a uMAP projection of all T cells colored by the expression of EOMES, KLRG1, CMC1, ENTPD1 and CXCL13. Darker shades indicate higher expression. FIG. 8G shows a RNAscope assay quantification boxplots (top). Each data point represents the fraction of the double positive CD8A/GZMK or CD8A/GZMB cells in stroma or tumor relative to the total number of CD8A/GZMK or CD8A/GZMB positive cells in tumor and stroma. Representative images of CD8A/GZMB (bottom left) and CD8A/GZMK (bottom right) co-hybridization. Blue: CD8A. Pink: GZMB or GZMK. Arrows are highlighting double positive cells. T: Tumor area. S: Stroma area. FIG. 8H shows a stacked bar plot with the fraction of CD8+ GZMK T cells relative to all CD8+ T cells in green and the fraction of CD8+ GZMB T cells relative to all CD8+ T cells in blue. Each bar represents one tumor, with the first 5 bars showing excluded tumors and the last 5 bars the infiltrated tumors. FIG. 8I shows boxplots of the per sample GZMK/CD8+ or GZMB/CD8+ score by immune phenotype calculated using the ICON7 (n=351), ROSiA (n=308) and TCGA (n=412) bulk RNA sequencing data. The statistical significance was determined using Wilcoxon test. The right panel shows bar graphs indicating the explained variance of the GZMB/CD8+ score, the GZMK/CD8+ score, or their combination in a logistic regression model predicting whether a sample has an excluded or infiltrated immune phenotype in each of the three datasets. Significant differences in the variance explained by each model was tested using a chi-squared test. FIG. 8J shows a cox proportional hazard model hazard ratio with 95% confidence interval for CD8+ score and GZMK/CD8+ score variables. Model based on ICON7 chemo arm patients with infiltrated and excluded tumors only. The bar graph shows explained variance of the single models with CD8+ score or GZMK/CD8+ score only, compared to the additive model with CD8+ score and GZMK/CD8+ score. Statistical difference of the models was tested using a chi-squared test. * p<0.05; ** p<0.01; *** p<0.001; **** p<0.0001.

FIGS. 9A-H show cellular and functional characterization of T cells infiltrating ovarian tumors. FIG. 9A shows a stacked bar plot with the fraction of each T cell population relative to the total T cell count in each patient. FIG. 9B shows bar plots with the fraction of CD4+IL7R cells, CD4+ CXCL13 and CD4+ FOXP3 cells compared to total CD4+ T cell count grouped by tumor immune phenotype. FIG. 9C shows bar plots with the fraction of Tgd/NK FGFBP2 and CD8+ FGFBP2 cells relative to the total T cell count grouped by immune phenotype. FIG. 9D shows a uMAP projection of all T cells colored by the gene expression of selected effector and memory T cell markers. Darker shades indicate higher expression. FIG. 9E shows a multiplex IF quantification boxplot. Each data point represents the fraction of triple positive CD3+ GZMB+PD-1+ cells relative to the double positive CD3+ GZMB+ cells in the tumor or stroma area. Representative IF images of Excluded (top) and Infiltrated (bottom) tumors. Dark blue: DAPI, light blue: CD3, pink: Granzyme B (GZMB), yellow: PD-1, green: PD-L1 and red: PanCK. Yellow arrows represent double positive CD3+ GZMB+ cells, and green arrows the triple positive CD3+ GZMB+PD-1+ cells. FIG. 9F shows RNAscope assay quantification boxplots. Each data point represents the fraction of the total stroma+tumor double positive CD8A/GZMK or CD8A/GZMB cells relative to the total number of CD8A in tumor and stroma. FIG. 9G shows boxplots with the per patient CIBERSORT CD8+ T cell signature z-score by immune phenotype for the bulk RNA sequencing data of the 15 ovarian cancer samples that have been single cell profiled for this study, the ICON7 and ROSiA clinical trial datasets and TCGA. GZMB and GZMK gene expression has been excluded from the signature z-score calculation. FIG. 9H shows a cox proportional hazard model hazard ratio with 95% confidence interval for CD8+ score, GZMB/CD8+ score and GZMK/CD8+ score variables. Model based on ICON7 chemo arm patients with infiltrated and excluded tumors only. For FIGS. 9B-C, significance was determined by t-statistic accounting for the patient variability as a random effect. For FIGS. 9E-G, significance was determined by a Wilcoxon test. * p-value<0.05, **** p-value<0.0001. n.s: non-significant.

FIGS. 10A-D show association of fibroblast phenotypes with the localization of T cells. FIG. 10A shows a uMAP projection of fibroblasts from all patients colored by identified cell populations. FIG. 10B shows a uMAP projection of all fibroblasts colored by the signature score expression. Signature score expression was derived as previously described in Dominguez, C. X et al., Cancer Discovery, 10:232-253 (2020); and Elyada, E. et al., Cancer Discovery, 9:1102-1123 (2019). FIG. 10C shows a heatmap of z-scored gene expression of the top 20 markers of each fibroblast population. Z-scored gene expression was calculated based on scran normalized and per cell population averaged expression values. FIG. 10D shows bar plots with the fraction of TGFB CAF and IL1 CAF cells compared to total fibroblast count grouped by tumor immune phenotype. Each single dot represents a patient. Significance was determined by the t-statistic accounting for the patient variability as a random effect.

FIGS. 11A-B show fibroblast populations in ovarian cancer. FIG. 11A shows a violin plot of the per cell CAF signature score distribution grouped by identified uMAP clusters. CAF signature scores were derived as previously described in Dominguez, C. X. et al., Cancer Discovery, 10:232-253 (2020); and Elyada, E. et al., Cancer Discovery, 9:1102-1123 (2019). FIG. 11B shows a stacked bar plot with the fraction of each fibroblast population relative to the total fibroblast count in each patient.

FIGS. 12A-F show phenotypically and functionally diverse subsets of myeloid cells linked to different tumor immune phenotypes. FIG. 12A shows a uMAP projection of myeloid cells from all patients colored by identified cell populations. FIG. 12B shows a uMAP projection of all myeloid cells colored by the expression of selected cell population marker genes. Darker shades indicate higher expression. FIG. 12C shows a diffusion map projection of all monocytes/macrophages from all patients colored by the identified monocyte/macrophage population. The diffusion map was computed based on the bbknn corrected data projection. FIG. 12D shows a uMAP projection of myeloid cells colored by signature scores of the TAM-like and MDSC-like myeloid subsets from Zhang, Q. et al. Cell, 179, 829-845 e820 (2019). Violin plot with per cell signature scores grouped by identified macrophage/monocyte populations. FIG. 12E shows a uMAP projection of all myeloid cells colored by TREM1 and TREM2 expression. Darker shades indicate higher expression. FIG. 12F shows bar plots with the fraction of the combined CD169/CX3CR1 macrophages and MARCO macrophages/FCN1 monocytes relative to the total monocyte and macrophage cell count. Significance was determined by using a Wilcoxon tank sum test.

FIGS. 13A-D show characterization of myeloid cell subsets in ovarian cancer. FIG. 13A shows a uMAP projection of myeloid cells of all patients colored by the expression of selected myeloid cell population markers. Darker shades indicate higher expression. FIG. 13B shows a heatmap of z-scored gene expression of the top 20 markers of each identified monocyte/macrophage population. Z-scored gene expression was calculated based on scran normalized and per cell population averaged expression values. FIG. 13C shows a violin plot of CIBERSORT monocyte and macrophage signature scores by cell based on the scran normalized expression values. Violin plots are grouped by identified macrophage/monocyte populations. FIG. 13D shows a stacked bar plot with the fraction of each monocyte/macrophage population relative to the total monocyte/macrophage count in each patient.

FIGS. 14A-G show tumor immune phenotypes shaped by cross-compartment interactions. FIG. 14A shows a dot plot of CXCL16 and CXCR6 expression by compartment and cell type. FIG. 14B shows a boxplot of the average CXCL16 scran normalized expression in tumor cells for each patient, grouped by immune phenotype. FIG. 14C shows a dot plot of the CXCR3 and corresponding chemokine ligand expression by cell type with heatmap depicting the enrichment of the corresponding cells in the three tumor immune phenotypes. FIG. 14D shows dot plot of the average expression of CXCL14, CXCL12, and CXCR4 in immune and stromal populations as well as in the tumor cell compartment with heatmap depicting the enrichment of the corresponding cells in the three tumor immune phenotypes. FIG. 14E shows a dot plot CX3CR1 and corresponding chemokine ligand expression by cell type. FIG. 14F shows a box plot of CX3CR1 expression by myeloid cell type. FIG. 14G shows model of cross-compartment chemokine ligand-receptor interactions in the context of the TME of each immune phenotype. The model for fibroblasts is depicted based on a trend for higher IL1 CAFs in infiltrated tumors. For FIGS. 14A, C, D and E, the color intensity of each dot indicates the average scran-normalized expression across all patients; the size represents the percentage of cells that express a gene compared to the total number of cells in that group. For FIGS. 14B and F, each dot indicates the average expression level for each patient. Statistical significance was calculated using a Wilcoxon test.

FIGS. 15A-I show chemokine ligand-receptor analysis across tumor, immune, and stromal compartments. FIG. 15A shows a boxplot of the CXCL16 expression by immune cell population. FIG. 15B shows a scatter plot of the CXCR6 expression in T cells vs CXCL16 expression in tumor cells colored by immune phenotype. FIG. 15C shows a scatter plot of the CXCR3 expression in all immune cells vs CXCL19, CXCL10 or CXCL11 expression in monocytes/macrophages colored by immune phenotype. Dot size indicates the relative fraction of CD169 macrophages compared to all monocytes/macrophages. FIG. 15D shows a dotplot of CXCR5 and CXCL13 expression by cell type. FIG. 15E shows a scatter plot of the CXCR5 expression in all B-TILs vs CXCL13 expression in CD4+ T cells colored by immune phenotype. Dot size indicates the relative fraction of CD4+ CXCL13 T cells compared to all CD4+ T cells. FIG. 15F shows a scatter plot of the CXCR4 expression in all immune cells vs CXCL12 or CXCL14 expression in fibroblasts colored by immune phenotype. Dot size indicates the relative fraction of IL1 CAFs compared to all fibroblasts. FIG. 15G shows a scatter plot of the CX3CR1 expression in all myeloid cells vs CCL26 or CX3CL1 expression in stromal cells colored by immune phenotype. FIG. 15H shows a dotplot of CCR7 and corresponding chemokine ligand expression by cell type. Boxplot of the average CCL21 scran normalized expression in endothelial cells for each patient, grouped by immune phenotype. Statistical significance was calculated using a Wilcoxon test. FIG. 15I shows a scatter plot of the CCR7 expression in B-TILs vs CCL21 expression in endothelial cells colored by immune phenotype. In FIGS. 15D and 15H, the color intensity of each dot indicates the average scran-normalized expression across all patients; the size represents the percentage of cells that express a gene compared to the total number of cells in that group.

FIG. 16 shows violin plots showing the distribution of the per cell antigen presentation and oxidative phosphorylation signature score for each patient. Z-scored gene expression was calculated based on scran normalized expression values.

FIGS. 17A and 17B show cellular and functional characterization of T cells infiltrating ovarian tumors. FIG. 17A shows a violin plot of the per cell Dysfunction and Exhaustion score grouped by identified T cell populations. Signatures scores have been derived from Li H. et al., Cell, 176, 775-789 e718 (2019) and Yost, K. E. et al., Nature Medicine, 25, 1251-1259 (2019). Significance was determined by t-statistic accounting for the patient variability as a random effect. FIG. 17B shows a box plot of TCF7 expression in each T cell subpopulation. Each dot represents the mean TCF7 expression in one patient and T cell subpopulation. Significance was determined by a Wilcoxon test.

FIG. 18 shows characterization of myeloid cell subsets in ovarian cancer. FIG. 18 shows a uMAP projection of DC cells of all patients after bbknn correction. Cells are colored by their identification DC subpopulation cluster.

FIG. 19 shows characterization of myeloid cell subsets in ovarian cancer. FIG. 19 shows a heatmap of z-scored gene expression of the top 20 markers of each identified DC subpopulation. Z-score gene expression was calculated based on scran normalized and per cell population averaged expression values.

### DETAILED DESCRIPTION

### I. Definitions

Unless stated otherwise, the following terms and phrases as used herein are intended to have the following meanings:

The term “near absence of T cells” as used herein refers to the amount of T cells present that includes from very low amounts (less than 20% of tumor area occupied by T cells and the T cell density is 0 out of a pathologist-defined T cell relative density range of 0-3) to undetectable amounts or amounts under the limit of detection (LOD) using conventional methods or measurement or detection.

An “effective amount” of an agent, e.g., a pharmaceutical composition, refers to an amount effective, at dosages and for periods of time necessary, to achieve the desired therapeutic or prophylactic result.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

All numbers in the specification and claims are modified by the term “about”. This means that each number includes minor variations as defined 10% of the numerical value or range in questions.

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### II. Diagnostic Methods for Designing a Treatment Protocol

The present application includes diagnostic methods for ovarian cancer in human patients. In some embodiments, the methods comprise designing a treatment protocol based on these diagnostic methods. The methods relate the expression of at least one of granzyme K (GZMK), granzyme B (GZMB) and the Triggering Receptor Expressed on Myeloid Cells (TREM) proteins TREM1 and TREM2 in a tumor sample from the patient.

In some embodiments, the method comprises evaluating all of GMZK, TREM1, and TREM2 in a tumor sample from the patient. In some embodiments, the method comprises evaluating all of GMZB, TREM1, and TREM2 in a tumor cell from the patient.

Thus, in some embodiments, the tumor sample may be a tumor cell. The tumor cell may be a stromal or immune cell from the tumor. In some embodiments, the tumor sample may be a tumor biopsy.

A. GZMK Expression

CD8+ T cells function in the defense response against tumor development and viral or bacterial infection. In some embodiments, CD8+ T cell quantity is associated with progress-free survival in cancer.

Upon binding of a surface antigen, CD8+ T cells may differentially express genes that encode key effector molecules of cytotoxicity. Granzymes are a group of effector molecules that are expressed to recognize, bind, and lyse target cells. Examples of granzymes include granzymes A, B, H, K, and M (GZMA, GZMB, GZMH, GZMK, and GZMM). In many embodiments, GZMK expression is elevated in CD8+ T cells in excluded tumor cells. In some embodiments, GZMK-expressing CD8+ T cells (GZMK/CD8+ T cells) are found in infiltrated and desert tumor cells. In some embodiments, GZMK/CD8+ T cells are nearly absent in infiltrated and desert tumor cells.

In many embodiments, methods of designing a treatment protocol for a human patient with ovarian cancer comprises determining the expression level of GZMK. In some embodiments, the method comprises determining GZMK expression level in a tumor sample from the patient. In some embodiments, the tumor sample comprises cells of the TME, the immune infiltrated phenotype, the immune excluded phenotype, and/or the immune desert phenotype.

In many embodiments, the methods further comprise comparing the expression level of GZMK to the expression level in a reference sample, as described below in Section II.E. In some embodiments, higher expression of GZMK when compared to a reference sample is associated with reduced survival. In some embodiments, increased level of GZMK compared to a reference sample is associated with excluded tumors.

B. GZMB Expression

In some embodiments, GZMB expression is elevated in CD8+ T cells instead of GZMK. In some embodiments, higher expression of GZMB when compared to a reference sample, as described below in Section II.E, is associated with the infiltrated tumor phenotype.

C. TREM1 Expression

In many embodiments, methods of designing a treatment protocol for a human patient with ovarian cancer comprises determining the expression level of TREM1 in a tumor sample from the patient. High TREM1 expression in macrophages infiltrating human tumors is associated with aggressive tumor behavior and poor patient survival. Pharmacological inhibition of TREM1 may provide survival advantage and protection from organ damage or tumor growth by attenuating inflammatory responses.

In many embodiments, the method comprises comparing TREM1 expression level to a reference sample, as described below in Section II.E. In some embodiments, an increased level of TREM1 expression compared to a reference sample is associated with the presence of MDSC-like myeloid cells. In some embodiments, increased level of TREM1 compared to a reference sample is associated with desert tumors.

D. TREM2 Expression

In many embodiments, methods of designing a treatment protocol for a human patient with ovarian cancer comprises determining the expression level of TREM2 in a tumor sample from the patient. TREM2 acts as a tumor suppressor in hepatocellular carcinoma and colorectal cancer. TREM1 and TREM2 are each linked to different subsets of myeloid cells and may inform treatment selection for ovarian cancer.

In many embodiments, the method comprises comparing TREM2 expression level to a reference sample, as described below in Section II.E. In some embodiments, an increased level of TREM2 expression compared to a reference sample is associated with the presence of TAM-like macrophages. In some embodiments, increased level of TREM2 compared to a reference sample is associated with excluded and/or infiltrated tumors.

E. Use of Reference Samples

In many embodiments, expression levels of GZMK, GZMB, TREM1, and/or TREM2 is compared to a reference sample.

In some embodiments, the reference sample is a healthy subject. In some embodiments, the reference sample is normal tissue, such as from the same part of the body. The normal tissue may be from a healthy subject or pool of healthy subjects, the same patient, a different patient, or a pool of patients.

In some embodiments, the reference sample is a patient who has a known desert tumor, a known excluded tumor, or a known infiltrated tumor. A reference sample may refer to cells across the TME of a patient tumor sample, or cells with a specific immune phenotype, such as infiltrated, excluded, or desert. In some embodiments, the reference sample is a sample of infiltrated cells, excluded cells, and/or desert cells. In some embodiments, the reference sample is a tumor sample obtained at a particular time point in a subject's medical history or a patient's treatment regimen. In some embodiments, the reference sample may be from the same patient, a different patient, or a pool of patients.

In some embodiments, the reference sample is a patient who has responded to therapy.

In some embodiments, the reference sample is data compiled across a plurality of patients and/or subjects. In some embodiments, the reference sample will be a pool of patient tumor tissue samples with pre-validated threshold for high vs. low for each of the GZMK, TREM1, and TREM2 gene expression levels relative to a reference gene or genes (i.e., housekeeping genes).

F. Gene Expression Profiling

Various technologies may be used to measure the expression levels of GZMK, GZMB, TREM1, and/or TREM2 from a patient tumor sample. In some embodiments, the expression levels are determined using a DNA or RNA sequencing method. In some embodiments, the method comprises measuring mRNA or RNA transcripts. In some embodiments, the RNA transcripts are determined by scRNAseq. The measured levels of mRNA transcripts of GZMK, GZMB, TREM1, and/or TREM2 may be normalized against the mRNA transcripts of at least one reference gene (i.e., housekeeping gene) in the tumor sample.

In some embodiments, the increased normalized level of RNA transcripts of GZMK is in CD8+ T cells. In some embodiments, the number of CD8+ T cells that are GZMK positive are greater than the number of CD8+ T cells that are GZMK negative. In some embodiments, the increase normalized level of RNA transcripts of TREM2 is in macrophages.

In some embodiments, the expression levels of GZMK, GZMB, TREM1, and/or TREM2 is determined using immunohistochemistry.

In some embodiments, gene expression profiling may comprise a cell sorting technique, such as fluorescence-activated cell sorting (FACS).

In some embodiments, the tumor sample may be separated into tumor, stromal, and immune cells before evaluating the expression level of at least one of GZMK, GZMB, TREM1, and/or TREM2.

### III. Methods of Characterizing an Ovarian Cancer as a Desert, Excluded, or Infiltrated Type of Tumor

The methods of the invention include methods of characterizing an ovarian cancer in a human patient according to the immune phenotypes: desert, excluded, and infiltrated. The methods may comprise determining the expression level of at least one of GZMK, GZMB, TREM1, and TREM2 in a tumor sample from the patient. As described above in Section II, each gene expression level is compared to the expression level in a reference sample. The characterization methods disclosed herein may be used to inform methods of treating a human patient with ovarian cancer. In some embodiments, a method of treatment comprises characterizing an ovarian cancer as desert, excluded, or infiltrated.

In some embodiments, higher expression of GZMK when compared to the reference sample is associated with the excluded tumor phenotype.

In some embodiments, higher expression of GZMB when compared to the reference sample is associated with the infiltrated tumor phenotype.

In some embodiments, higher expression of TREM1 when compared to the reference sample is associated with desert tumor phenotype.

In some embodiments, higher expression of TREM2 when compared to the reference sample is associated with excluded and infiltrated tumor phenotypes.

Thus, these associations allow a clinician to characterize an ovarian cancer as desert, excluded, or infiltrated, either as the only point of data or in conjunction with other points of data.

### IV. Methods of Treatment

The methods of the invention include methods of treatment of ovarian cancer in human patients. In many embodiments, the methods comprise determining the expression level of at least one of GZMK, GZMB, TREM1, and TREM2 in a tumor sample from the patient, as described above in Section II. In many embodiments, the methods comprise comparing the expression level of at least one of GZMK, TREM1, and TREM2 to expression level in a reference sample, as described above in Section II.E.

In some embodiments, the methods of characterizing an ovarian cancer as desert, excluded, or infiltrated, as described above in Section III, may inform the methods of treatment. The characterization methods comprise determining the expression level of at least one of GZMK, GZMB, TREM1, and TREM2 in a tumor sample from the patient. As described above in Section II, the expression of each gene is compared to a reference sample.

A. Methods of Treating Tumors with Lower GZMK Expression Associated with Increased Patient Survival

In some embodiments, lower GZMK expression when compared to the reference sample is associated with increased patient survival. In some embodiments, the methods comprise administering to the patient chemotherapy. In some embodiments, the chemotherapy comprises Albumin bound paclitaxel (nab-paclitaxel (Abraxane®)), altretamine (Hexalene), Capecitabine (Xeloda®), carboplatin, cisplatin, cyclophosphamide (Cytoxan®), docetaxel (Taxotere®), etoposide (VP-16), gemcitabine (Gemzar®), ifosfamide (Ifex®), irinotecan (CPT-11 (Camptosar®)), doxorubicin (such as liposomal doxorubicin (Doxil®)), melphalan, paclitaxel (Taxol®), pemetrexed (Alimta®), topotecan, vinorelbine (Navelbine®), Niclosamide, Metformin, BAY 87-2243, Decitabine, Guadecitabine, Azacytidine, Abagovomab, Oregovomab, NeoVax with Nivolumab, Anlotinib, Enoxaparin with Rosuvastatin, Niraparib, Chiauranib, Trabectedin with pegylated liposomal Doxorubicin, ACB-S6-500, SGI-110, Letrozole, Pazopanib, Palbociclib, Apatinib, Masitinib, Cabazitaxel, IMAB027, Fludarabine, ABT-888, Fostamatinib, Olaparib, Temozolomide, Talazoparib, P53-SLP, OMP-54F28, Hydralazine and magnesium valproate, Fludarabine, Lapatinib, Bendamustine HCL, Sorafenib, Camrelizumab, Tremelilumab, Tocotrienol, and/or Exemestane.

B. Methods of Treating Tumors with Higher GZMK Expression Indicating an Excluded Tumor

In some embodiments, higher GZMK expression is associated with reduced patient survival and/or indicates an excluded tumor. In some embodiments, the methods comprise administering to the patient chemotherapy. In some embodiments, higher GZMK expression is associated with reduced patient survival. In some embodiments, chemotherapy is stopped after GZMK expression has been shown in the tumor. In some embodiments, palliative care is given to the patient after GZMK expression has been shown in the tumor.

C. Methods of Treating Tumors with Higher TREM1 Expression Indicating a Desert Tumor

In some embodiments, higher TREM1 expression indicates a desert tumor. In some embodiments, the tumor is an ovarian tumor. In some of these embodiments, the treatment methods comprise treating the patient with chemotherapy or autologous/allogenic effector cells. In some embodiments, the chemotherapy comprises Albumin bound paclitaxel (nab-paclitaxel (Abraxan®)), altretarmine (Hexalen®), Capecitabine (Xeloda®), carboplatin, cisplatin, cyclophosphamide (Cytoxan®), docetaxel (Taxotere®), etoposide (VP-16), gemcitabine (Gemzar®), ifosfamide (Ifex®), irinotecan (CPT-11 (Camptosar®)), doxorubicin (such as liposomal doxorubicin (Doxil®)), melphalan, paclitaxel (Taxol®), pemetrexed (Alimta®), topotecan, vinorelbine (Navelbine®), Niclosamide, Metformin, BAY 87-2243, Decitabine, Guadecitabine, Azacytidine, Abagovomab, Oregovomab, NeoVax with Nivolumab, Anlotinib, Enoxaparin with Rosuvastatin, Niraparib, Chiauranib, Trabectedin with pegylated liposomal Doxorubicin, ACB-S6-500, SGI-110, Letrozole, Pazopanib, Palbociclib, Apatinib, Masitinib, Cabazitaxel, IMAB027, Fludarabine, ABT-888, Fostamatinib, Olaparib, Temozolomide, Talazoparib, P53-SLP, OMP-54F28, Hydralazine and magnesium valproate, Fludarabine, Lapatinib, Bendamustine HCL, Sorafenib, Camrelizumab, Tremelilumab, Tocotrienol, and/or Exemestane.

In some embodiments, the treatment comprises a therapy targeting MDSC-like myeloid cells. In some embodiments, the MDSC-like myeloid cells are ovarian tumor cells. In some embodiments, the therapy targeting MDSC-like myeloid cells may comprise cisplatin, 5-flurouracil, gemcitabine, paclitaxel, a liver X receptor (LXR) beta agonist, a checkpoint inhibitor, and/or anti-TGFβ therapy (such as anti-TGFβ antibodies including Pembrolizumab and Fresolimumab). In some embodiments, the checkpoint inhibitor therapy is an anti-PD-1 therapy, such as pembrolizumab (Ketruda®), nivolumab (Opdivo®), cemiplimab (Libtayo®), Toripalimab, CT-011, monoclonal antibody HX0088, and antibody AK105. In some embodiments, the checkpoint inhibitor therapy is an anti-PD-L1 therapy, such as atezolizumab (Tecentriq®), avelumab (Bavencio®), durvalumab (MEDI4736 anti-PD-L1; Imfinzi®), Tremelimumab, mAb ZKAB001, Tremelimumab, and Ramucirumab (Cyramza).

D. Methods of Treating Tumors with Higher GZMB and/or Higher TREM2 Expression Indicating an Infiltrated Tumor

In some embodiments, higher GZMB expression and/or higher TREM2 expression indicates an infiltrated tumor. In some embodiments, the infiltrated tumor is an ovarian tumor. In some embodiments, the treatment comprises a therapy targeting TAM-like macrophage cells. In some embodiments, the treatment methods comprise treating the patient with cancer immunotherapy, such as, for example, checkpoint inhibitor therapy. In some embodiments, the checkpoint inhibitor therapy is an anti-PD-1 therapy, such as pembrolizumab (Ketruda®), nivolumab (Opdivo®), cemiplimab (Libtayo®), Toripalimab, CT-011, monoclonal antibody HX0088, and antibody AK105. In some embodiments, the checkpoint inhibitor therapy is an anti-PD-L1 therapy, such as atezolizumab (Tecentriq®), avelumab (Bavencio®), durvalumab (MEDI4736 anti-PD-L1; Imfinzi®), Tremelimumab, mAb ZKAB001, Tremelimumab, and Ramucirumab (Cyramza). In some embodiments, the cancer immunotherapy agent may comprise Durvalumab (MEDI4736 anti-PD-L1; Imfinzi®), motolimod, oncolytic virus, NY-ESO-1 cancer vaccine, anti-XBP1 therapy, anti-angiopoietin therapy, anti-DLL/Notch therapy, anti-HER2 therapy, anti-mesothelin therapy, anti-RANKL therapy, anti-TROP2 therapy, and VEGF/VEGF-R therapy.

E. Methods of Treating Tumors with Higher Expression of GZMB, TREM1, and/or TREM2

In some embodiments, the methods of treating tumors with higher expression of GZMB, TREM1, and/or TREM2 comprise treating the patient with immune effector cells. Examples of immune effector cells include T cell trafficking modulators, epigenetic modulators, TME remodeling molecules, and radiation therapy.

### EXAMPLES

The following are examples of methods and compositions of the invention. It is understood that various other embodiments may be practiced, given the general description provided above.

We transcriptionally dissect the tumor immunity continuum in human ovarian cancer, characterizing the composition of and cell-cell interactions in this complex ecosystem with scRNAseq profiling and immunohistochemistry. A total of 93,218 single cells from tumor tissues derived from 15 patients with newly diagnosed ovarian cancer are analyzed, enabling us to define cellular and functional phenotypes for the tumor, immune and stromal compartments in the context of the three immune phenotypes. In parallel, we employ in situ assays to characterize the spatial distribution of CD8+ GZMB and CD8+ GZMK T cells in the infiltrated and excluded tumors, and validate their differential enrichment in different immune phenotypes in multiple independent large bulk RNAseq clinical datasets from ovarian cancer patients. Finally, we identify potential crosstalk within and between these diverse cellular phenotypes in the context of chemokine ligand-receptor interactions. Our comprehensive single-cell profiling study provides additional insights into the biology that shapes the distinct tumor immune phenotypes, and it may inform new therapeutic strategies for shifting tumors along the immunity continuum and thereby optimizing the clinical benefits of cancer immunotherapies.

### Example 1. Single Cell Transcriptional Profiling Defines the Complete Cellular Ecosystem of Patient-Derived Primary Ovarian Tumors

### 1.1. Clinical Samples—Human Tumor Specimens, Procurement and Processing

KIYATEC sample collection. After providing written informed consent, patients >18 years of age with suspected or known ovarian cancer were enrolled onto an Institutional Review Board (IRB) approved biology protocol by Prisma Health, formerly known as Greenville Health System, Cancer Institute (IRB-Committee C). Tissue acquisition was carried out in accordance with the guidelines and regulations specified by Prisma Health and informed consent was obtained from all participants. Fresh tissue was collected at surgical debulking or laparoscopic biopsy of primary tumor sites.

In situ validation collection. An independent ovarian cancer tissue collection (n=17) was procured from Cureline, Inc (Brisbane, CA, US) for in situ validation. Procured samples also had an appropriate Institutional Review Board (IRB) approval.

### 1.2. CD8 Immunohistochemistry

Fresh tissue samples were fixed in formalin and embedded in paraffin and 10 μm sections were mounted onto glass slides. Rehydration and antigen retrieval were performed using Tris-EDTA buffer, pH 9.0 (Abcam, Cambridge, MA). Slides were stained with anti-CD8 [144B] (Abcam, Cambridge, MA) or IgG1 [B11/6] Isotype control (Abcam, Cambridge, MA) both at a dilution of 1:50 for 2 hours at room temperature. Staining was detected using Mouse and Rabbit Specific HRP/DAB IHC Detection kit (Abcam, Cambridge, MA). Images were taken on an Olympus IX70 microscope with a Jenoptik (Jena, Germany) ProgRes C14plus camera and ProgRes CapturePro software.

### 1.3. Cell Preparation for scRNAseq

Within 24 hours of surgery, ovarian tissues were received and immediately enzymatically dissociated into single cells which were cryopreserved in media containing 10% DMSO with the exception of exc5 for which the tissue was frozen before dissociation. This protocol has been validated by KIYATEC. Frozen tumor cell suspensions were thawed and diluted in FACS Stain buffer (1×PBS pH 7.4, 0.2% BSA, 0.09% NaAzide). Cells were counted on Cellometer Auto 2000 with the dual-fluorescence AO/PI. Then, the cells were incubated for 30 min with FcR blocking reagent followed by antibodies for another 30 min on ice. Immediately prior to sorting on FACSaria Fusion, cells were stained with live and dead markers 7-AAD ( 1/16) and Calcein Blue ( 1/500). Doublets were excluded and viable cells identified based on low 7-AAD and high Calcein blue. Antibodies used for sorting cells into a tumor, immune and stromal compartment per ovarian tissue were anti-CD45-APC-Cy7 ( 1/100, #304014, BioLegend) and anti-EpCAM-APC ( 1/20, #324208, BioLegend). After sorting, samples were immediately spun and resuspended in PBS at 600-1,000 cells/μL according to the cell count provided by the cell sorter. To prepare Mater Mix+cell suspension, we refer to the Cell Suspension Volume Calculator Table in Chromium Single Cell 3′ Reagent Kits User Guide (v2 Chemistry) (10× Genomics, California, USA) to add the appropriate volume of nuclease-free water and corresponding volume of single cell suspension (targeting cell recovery 5000-6000 cells) to Master Mix for a total of 100 μl in each tube. 90 μl of the above mixture were loaded in Chromium Chip B, subsequently gel beads and other reagents loaded in the chip according to the protocol (Gel Bead & Multiplex Kit V2 and Chip Kit (#PN-120237, 10× Genomics)). After running Chromium Controller for Gel Bead-In Emulsions (GEMs) generation and cell barcoding, GEMs were transferred to thermal cycler for GEMs reverse transcription incubation, followed by post GEMs-RT Cleanup, cDNA Amplification, QC and quantification.

### 1.4. Preparation of scRNASeq Libraries

For each patient, we generated one library per compartment (tumor, immune and stromal) resulting in three libraries per patient, with the exception of four desert tumors for which immune and stromal cells were not separated and two libraries per compartment (tumor and immune/stromal) were generated. 3′ Gene Expression Library Construction using the Chromium Single Cell 3′ Library (v2 chemistry) was performed according to manufacturer's instructions (support.10×genomics.com/single-cell-gene-expression/library-prep/doc/user-guide-chromium-single-cell-3-reagent-kits-user-guide-v2-chemistry). To reduce technical batch effects, we randomized the generation of libraries by both compartment and immune phenotype. Post library construction QC was done by Agilent Bioanalyzer High Sensitivity chip (#5067-4627, Agilent Technologies, Santa Clara, California, USA) and libraries were quantified by KAPA library quantification universal kit (#07960140001, Roche).

### 1.5. Sequencing and Raw Data Processing of scRNAseq Libraries

All libraries per patient were pooled and sequenced on an Illumina NextSeq500 with the High output kit v2 or v2.5 (150 cycles). We confirmed that both kit v2 and v2.5 generated similar results and no technical batch effects were detected comparing the sequencing results from both kits on the same library. Reads were mapped to the human genome (GRCh38) using CellRanger v3.0.2 (10×genomics.com). First, the cellranger mkfastq command with the cellranger sample sheet was used to demultiplex the base call files for each flow cell into fastq files. Second, the cellranger count command was called to generate single cell feature counts for each library by specifying the library name in the argument. The filtered feature barcode matrix was used for further data analysis.

### 1.6. Analysis of scRNA Sequencing Data

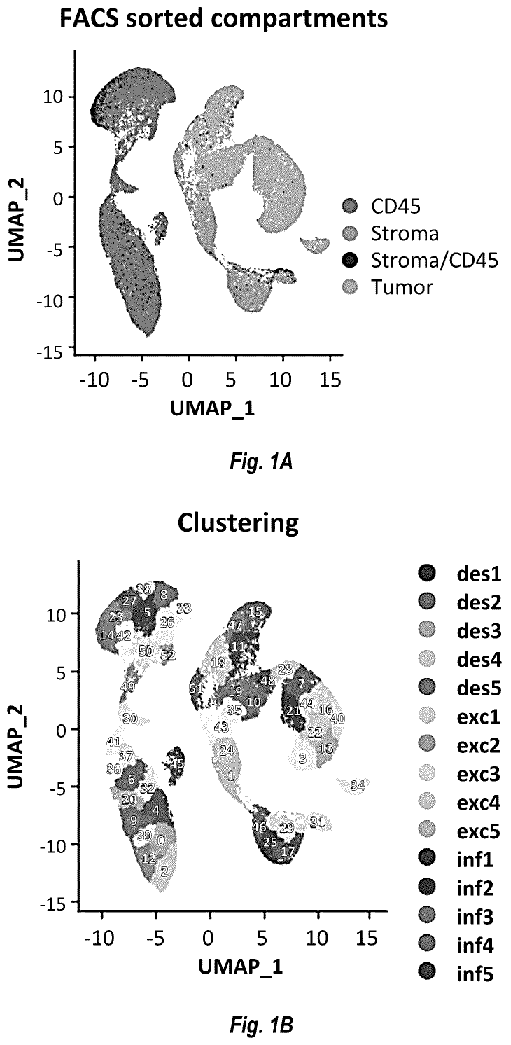

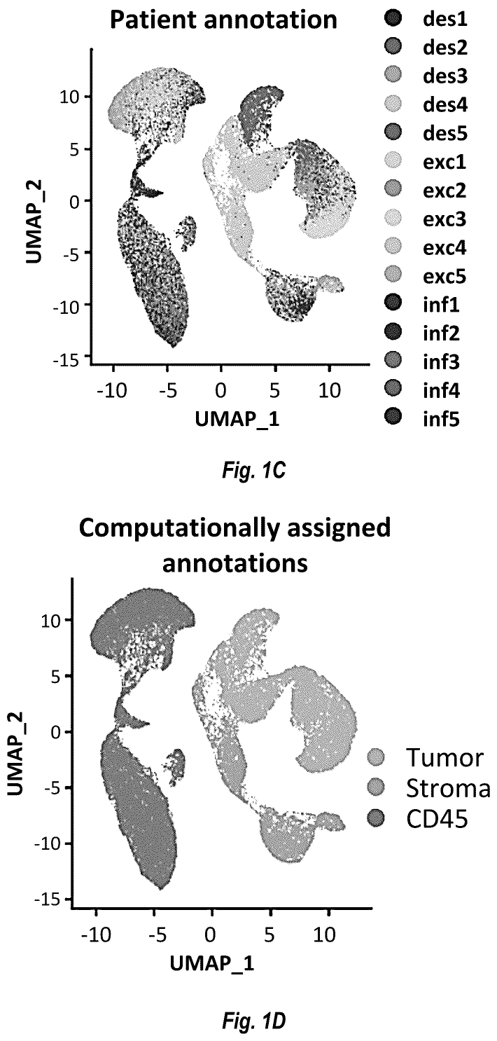

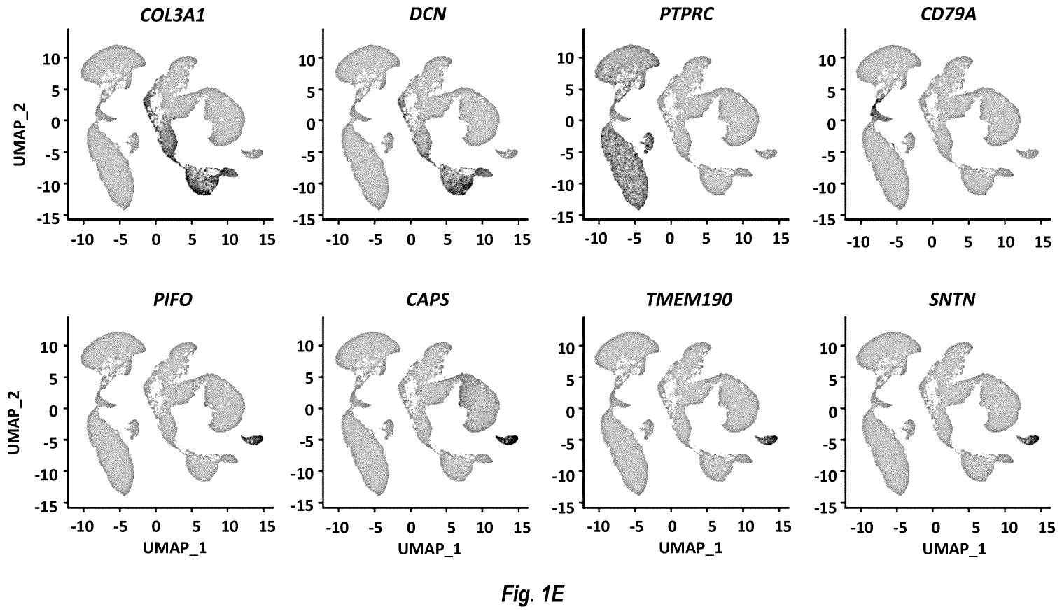

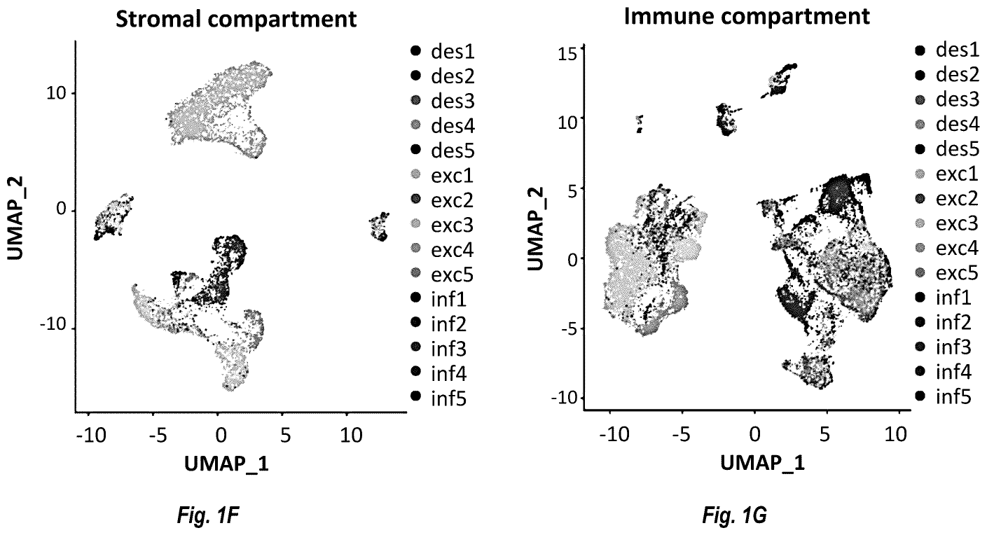

The core scRNAseq steps from CellRanger were performed using seurat v3.0.0. First, each library was converted into a seurat object using read10× and makeseuratobject. To perform filtering of the compartment annotations and to separate the desert stromal from desert immune cells that were sequenced in a pooled library, the data of all 44 libraries were merged (FIGS. 1A-E). The data was filtered to include genes that were expressed in at least 10 cells and cells that expressed at least 200 genes, not more than 6000 genes and less than 5% of mitochondrial transcripts. These cutoffs were determined through QC inspection of each library. Subsequently, the data was log normalized, variable genes detected through a mean-variance inspection, scaled and the principle components computed. The top principle components were identified using an elbow plot and used for the uMAP dimensionality reduction. Cluster identification was done at a resolution that would best separate the mixed stroma-tumor interface based on the FACS stromal and tumor annotation. To identify the immune, stromal, tumor and normal epithelial clusters, mean expression of the following gene markers for each cluster was calculated: 1) immune compartment: PTPRC, CD79A, 2) stromal compartment: VWF, PECAM1, COL1A1, COL3A1, DCN, 3) tumor compartment: EPCAM, 4) normal epithelial cells: PIFO, CAPS, TMEM190, SNTN. The cluster identity was determined at a mean expression cutoff greater than 0.8, which resulted in most concordant annotation with the FACS annotation. Cells that clustered with compartments other than their FACS annotated compartment and a cluster of normal epithelial cells (cluster 34) were removed from further analysis.

The raw data of each filtered and newly annotated compartment was processed separately for further downstream analysis as described above. The number of principal components for the dimensionality reduction was determined for each compartment individually. The major cell types in the stromal and immune compartments were defined through per cluster mean expression of the following gene markers: 1) fibroblasts: DCN, C1R, PDGFRA, OGN, 2) endothelial cells: PECAM1, 3) pericytes: RGS5, 4) B-TILs: MS4A1, 5) plasma cells: SDC1, 6) T cells: CD2, CD3E, 7) myeloid cells: CD14, CSF1R, LILRA4. For further analysis of the fibroblast, T cell and myeloid cell populations the data was subsetted to these cells and processed starting from the raw data as described above.

The analysis of each individual patient's T cell and myeloid cell population was performed by splitting the subsetted population by patient and processing the raw data as described above. Patient data with less than 50 myeloid or T cells were excluded from the single patient analysis. Positive cluster gene markers were identified using the seurat FindAllMarkers function and the Wilcoxon test. For the analysis of the fibroblast phenotypes, desert 4 fibroblasts were excluded since they are clustering separately from all other tumors.

Gene expression values plotted in uMAPs, heatmaps, violin plots, boxplots are scran normalized expression values calculated based on raw expression values (scran v1.10.2). Lun, A., Genome Biol. 1-14 (2016); doi:10.1186/s13059-016-0947-7 (2016).

### 1.7. Results

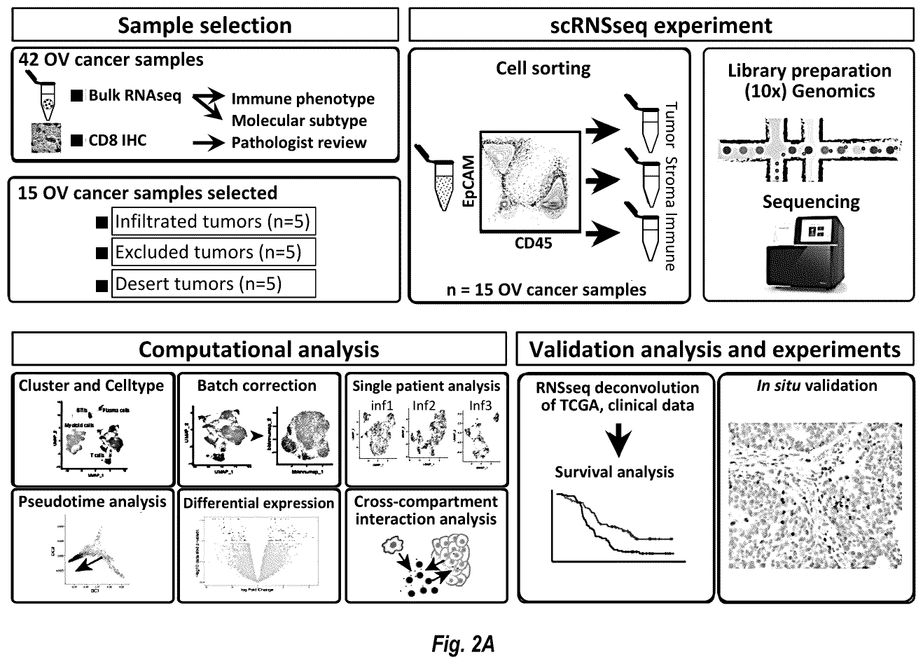

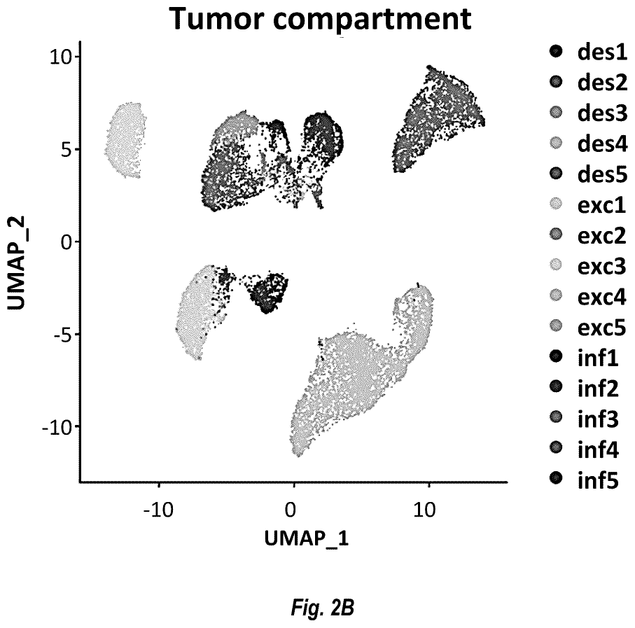

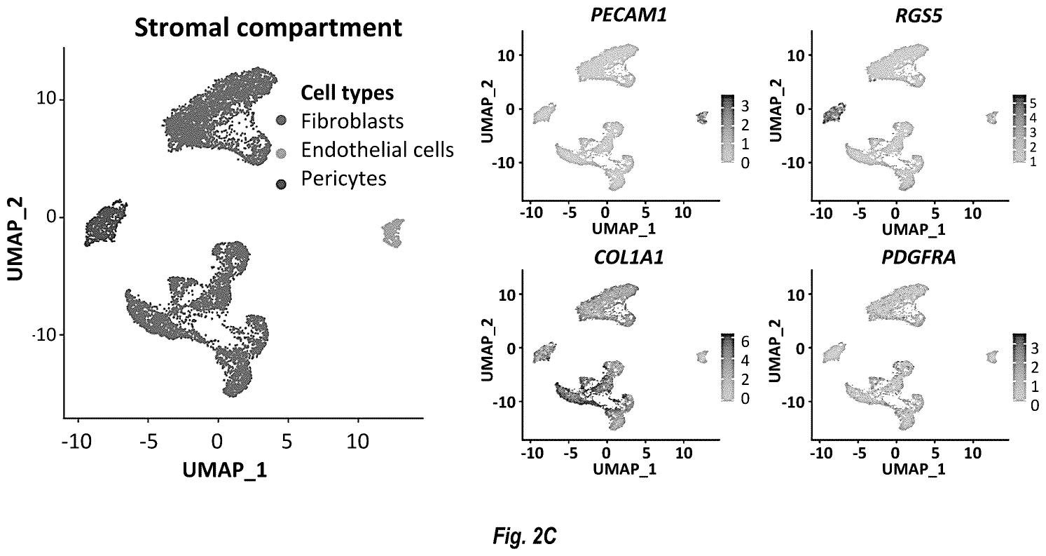

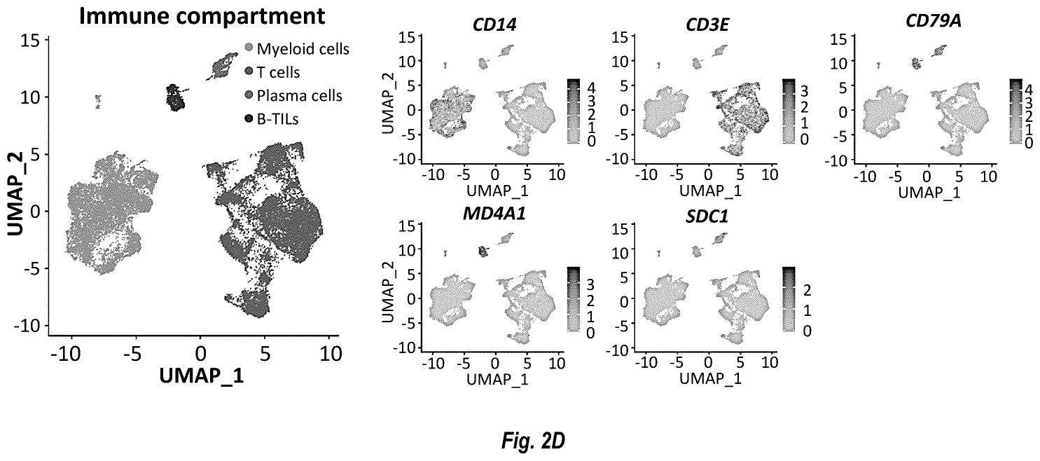

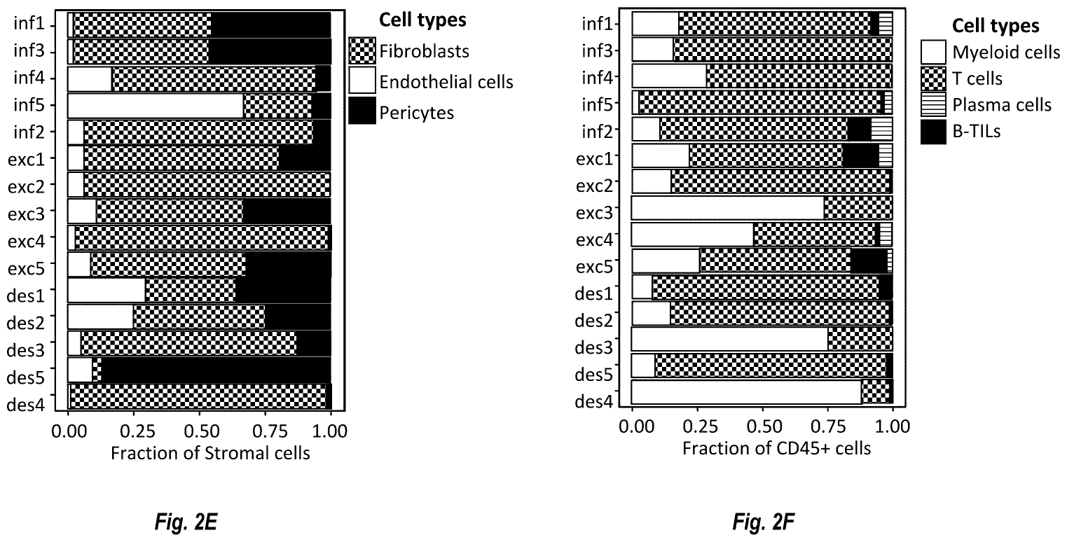

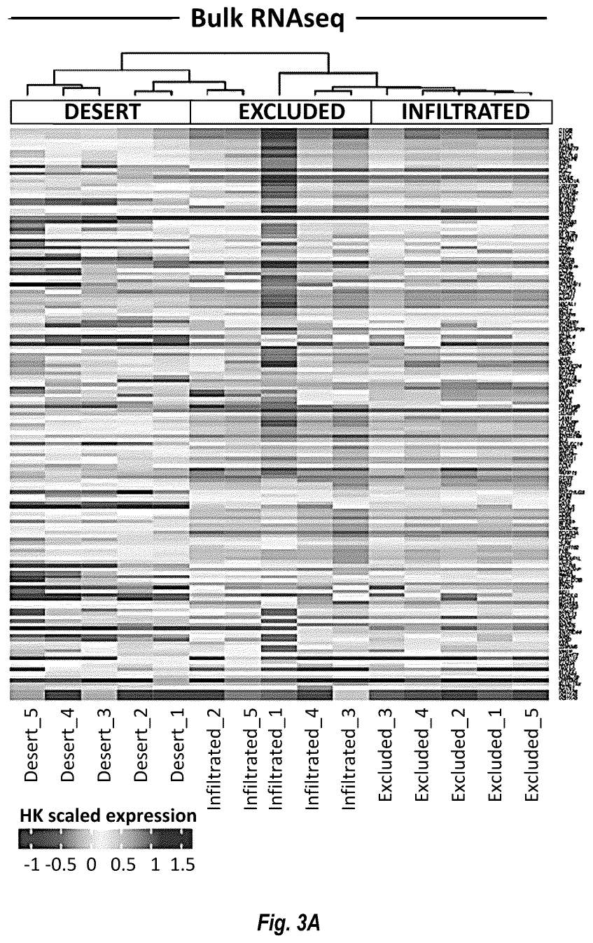

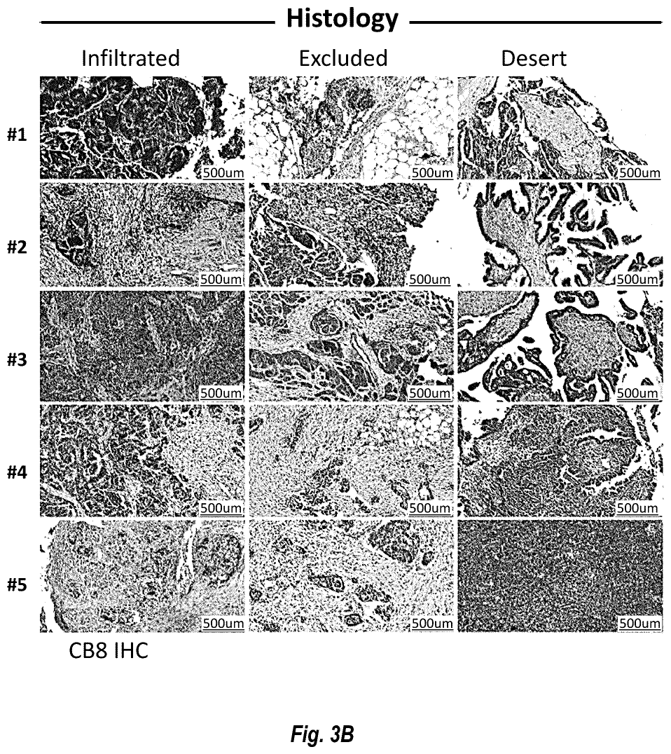

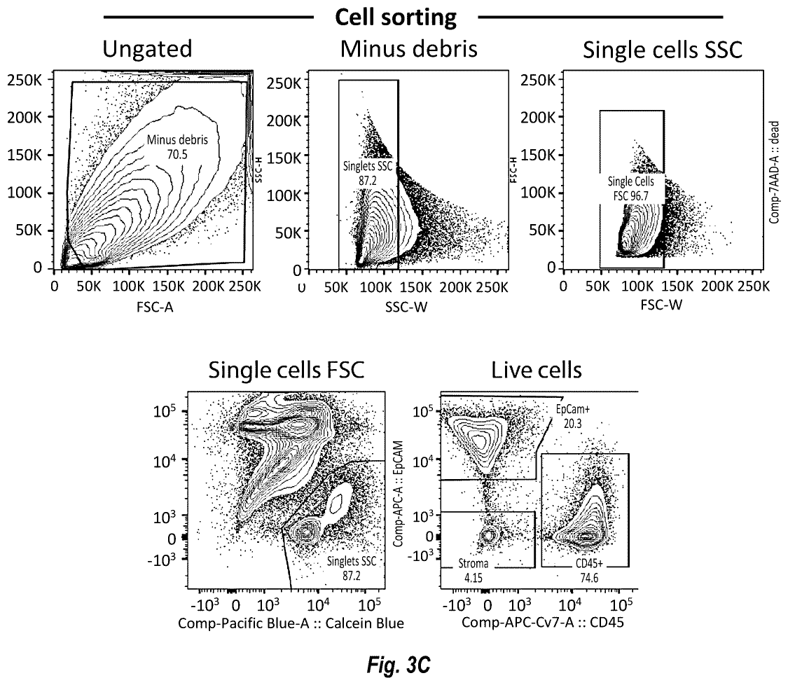

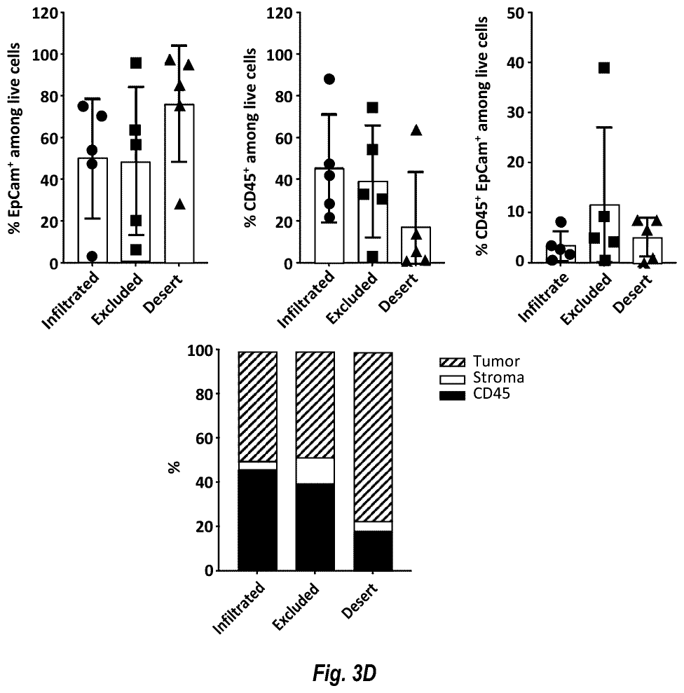

To dissect the landscape of the tumor immunity continuum in ovarian cancer, we performed RNAseq and CD8 IHC analysis on tumor samples collected from 42 newly-diagnosed ovarian cancer patients (FIG. 2A). The RNAseq data were utilized to predict the immune phenotype of each tumor (i.e. infiltrated, excluded or desert) with a previously developed, ovarian cancer-specific, transcriptional classifier (Desbois, M et al., Nature Communications, 11:5583, doi:10.1038/s41467-020-19408-2 (2020)) (FIG. 3A). In parallel, CD8+ T cell infiltration into the stroma and tumor epithelium was evaluated based on CD8 IHC staining (FIG. 3B). We employed both the classifier-based predictions and the CD8 IHC categorization to assign a tumor immune phenotype to each tumor sample (data not shown). Next, we selected a total of 15 ovarian tumors, with 5 tumors representative of each of the three immune phenotypes, and performed scRNAseq to further characterize the cellular composition associated with these phenotypes. To separate the tumor, stromal, and immune compartments, we used flow cytometry (FACS) to sort the tumor cells (EpCAM+CD45−), the stromal cells (EpCAM− CD45−) and the immune cells (EpCAM− CD45+) of each tumor sample (FIG. 3C). Each of the three compartments (tumor, immune, stromal) was subjected to scRNAseq as a separate library, with the exception of the desert stromal and immune cells (stromal/immune) which were pooled by patient due to the low fraction of CD45+ immune cells in desert tumors (FIG. 3D). In total, we sequenced 44 libraries resulting in 40,539 tumor cells, 35,296 stromal cells, 15,049 immune cells and 2,334 stromal/immune cells from the pooled libraries. Stromal and immune cell identities in the pooled desert tumor libraries were assigned computationally as described above in Example 1, Section 1.6 (FIGS. 1A-E).

Next, we analyzed each compartment separately to better characterize the heterogeneity and cellular composition. The tumor cells primarily clustered by patient, which likely reflects the strong interpatient heterogeneity shown in previous studies (FIG. 2B). Jerby-Arnon, L. et al., Cell, 175:984-997.e24 (2018); Puram, S. V. et al., Cell, 171:1644.el-1611.e24 (2017); Tirosh, I. et al., Science, 352:189-196 (2016). In contrast, cells from both the stromal and immune compartments clustered by cell type, with secondary clustering by patient to varying extent within the cell type clusters (FIGS. 2C, 2D, 1F, and 1G). We defined the cell types based on the expression of known cell type markers: fibroblasts (COL1A1, PDGFRA), endothelial cells (PECAM1) and pericytes (RGS5) in the stromal compartment; and myeloid cells (CD14), T cells (CD3E), plasma cells (CD79A, SDC1), as well as tumor-infiltrating B lymphocytes (B-TILs) (CD79A, MS4A1), in the immune compartment (FIGS. 2C and 2D). The relative prevalence of cell types within stromal and the immune compartments was highly variable between patients and did not show a clear association with the tumor immune phenotypes (FIGS. 2E and 2F).

### Example 2. Tumor-Intrinsic Features Associated with Different Tumor Immune Phenotypes

### 2.1. Pseudo-Bulk Differential Expression and Gene Set Enrichment Analysis

We used a pseudo-bulk approach to perform differential gene expression (DGE) analysis. For each sample, raw UMI counts for each gene were summed across cells of a cell population of interest derived from that sample, resulting in sample-level UMI counts. Samples with fewer than five cells and genes with less than 50 reads across samples were excluded from the analysis. We then calculated the size factors for each pseudo-bulk sample using calcNormFactors (edgeR) and used voom-limma to perform differential gene expression analysis on these sample-level pseudo-bulk expression profiles. For the G2/M corrected pseudo-bulk expression analysis in the tumor compartment, we calculated a G2/M score per cell using the CellCycleScoring function with G2/M cell cycle genes as previously described. Tirosh, I. et al., Science, 352:189-196 (2016). The per cell scores were averaged by patient and added to the voom-limma design matrix as a covariate. Gene set enrichment analysis was performed on the results of the differential gene expression analysis using the fgsea package and the hallmark gene set from the molecular signatures database collection using the msigdbr package. Subramaniam, A. et al., Proc. Natl. Acad. Sci. U.S.A., 102:15545-15550 (2005). In detail, the differentially expressed gene list was ranked according to the combined log fold change and adjusted p-value and used as an input for the gene set enrichment analysis.

### 2.2. Statistical Analysis

Statistical analysis was performed in R. The statistical methods used for each analysis are described within the figure legends. All boxplots report the 25% (lower hinge), 50%, and 75% quantiles (upper hinge). The lower whiskers indicate the smallest observation greater than or equal to lower hinge—1.5*interquartile range, the upper whiskers indicate the largest observation less than or equal to upper hinge+1.5*interquartile range as default in the geom_boxplot( ) function. Error bars in barplots represent the standard deviation.

### 2.3. Results

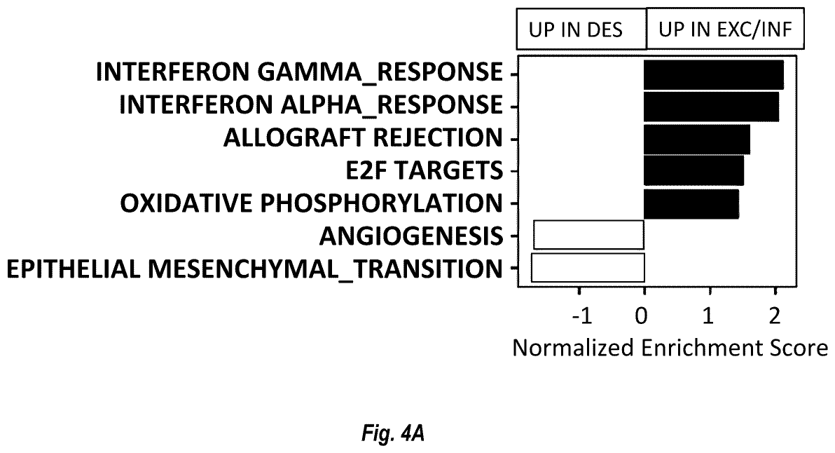

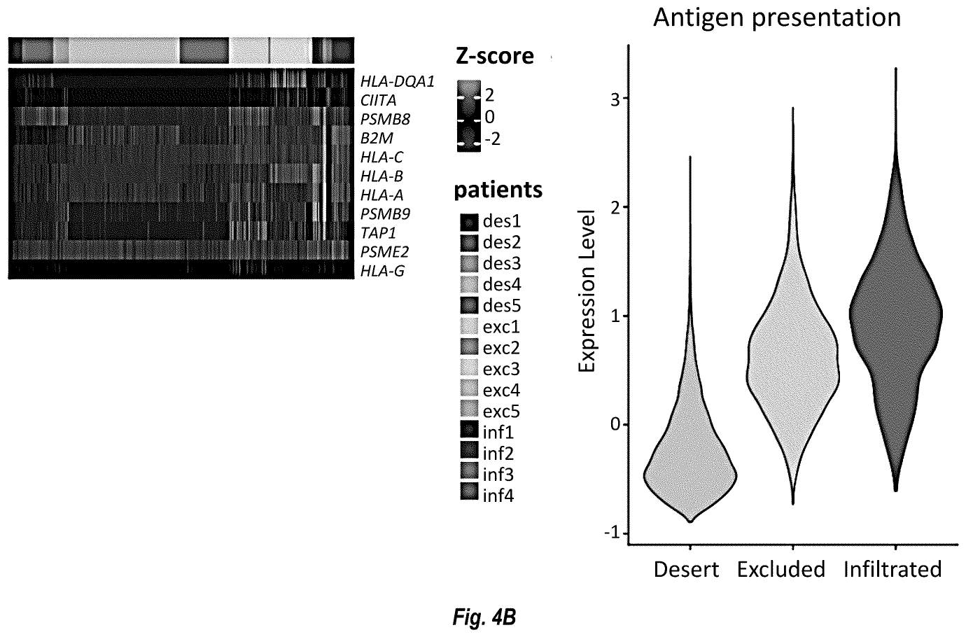

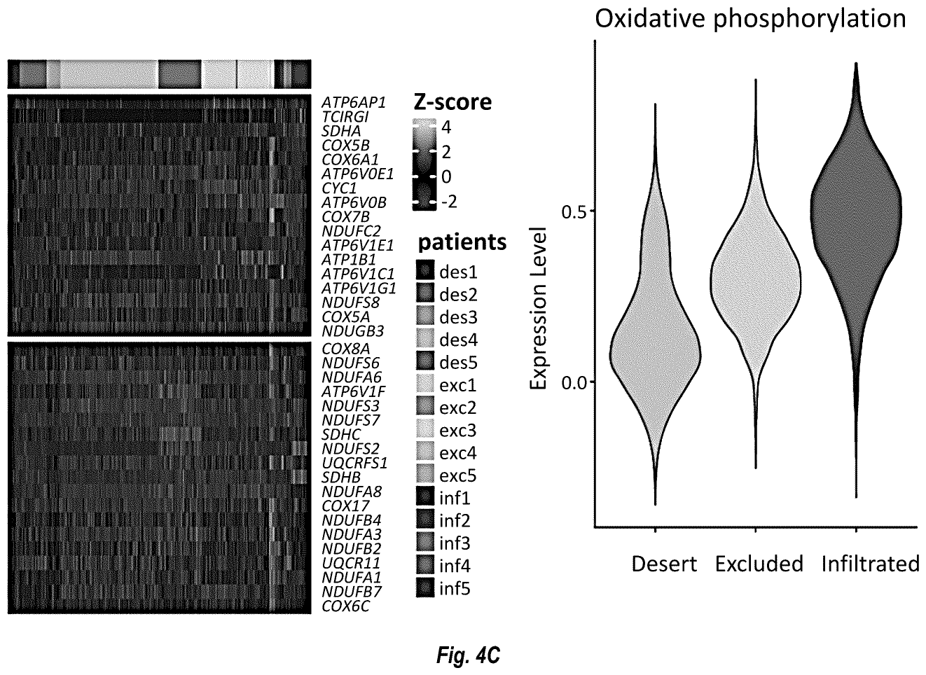

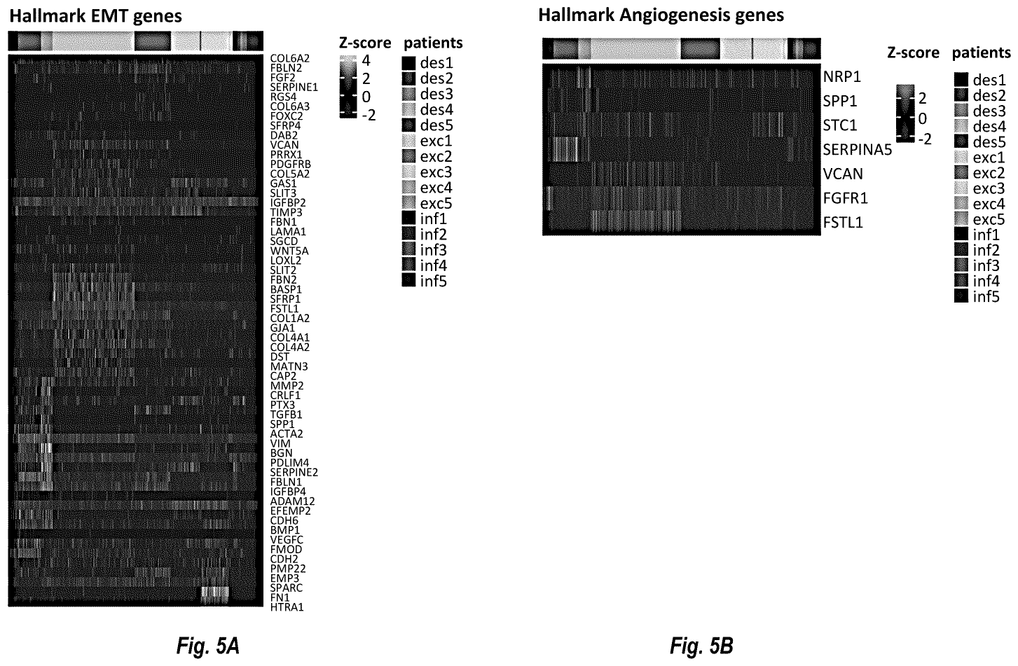

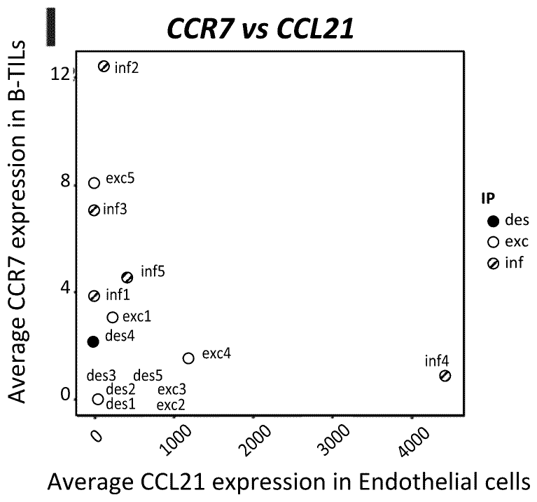

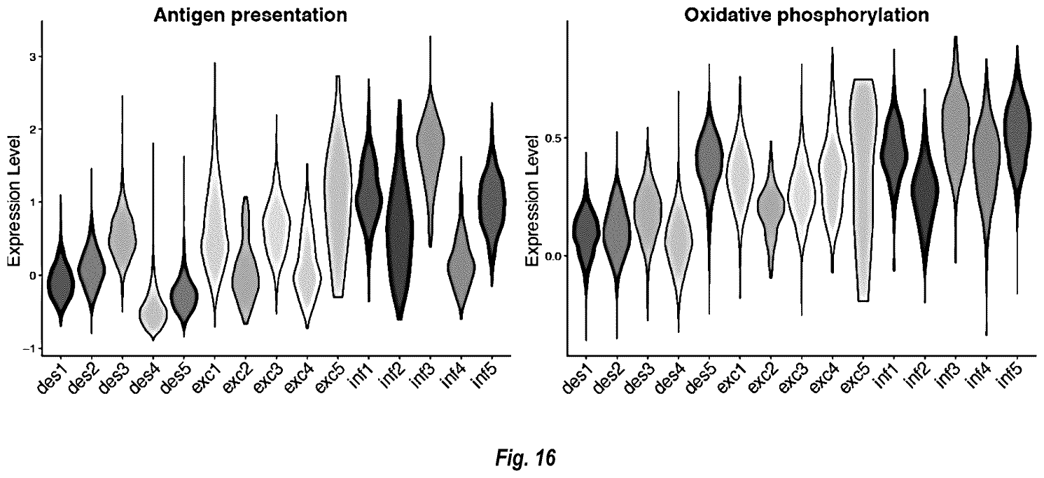

We first investigated whether tumor-intrinsic features contribute to patterns of immune infiltration. Using the scRNAseq data from the tumor cell compartment, we performed a pseudo-bulk differential expression analysis between the tumor immune phenotypes to identify differential expression patterns. Notably, there were no significant tumor-cell transcriptional differences between excluded versus infiltrated tumors (all adjusted p-value>0.05). This may be due to inter-tumor heterogeneity, even within an immune phenotype (FIG. 2B), which limits statistical power; alternately, it may suggest that the major drivers of CD8+ T cell exclusion vs. infiltration in the tumor epithelium lie within other cells in the TME, not the tumor cells themselves. In contrast, 29 genes were significantly differentially expressed between desert tumors and the combined set of infiltrated and excluded tumors. Gene set enrichment analysis revealed that these genes were mainly associated with proliferative pathways, and that their identification was driven by three desert tumors with a proliferative molecular subtype. Hence, to identify other biological processes, we accounted for the G2/M state. The G2/M-corrected analysis found genes involved in epithelial-mesenchymal transition (EMT) and angiogenesis to be significantly enriched in desert tumors (adjusted p-value=0.03 and 0.01, respectively, FIGS. 4A, 5A, and 5B; and Table 1). On the other hand, tumor cells of excluded and infiltrated tumors were significantly enriched in interferon response pathways (adjusted p-value=0.03), mainly driven by genes encoding machinery for MHC class I and II processing and presentation (e.g. HLA-A,B,C; B2M; HLA-DQA1; TAP1; PSMB8; PSMB9) (FIGS. 4A, 4B, and 16; and Table 1). Interestingly, we also observed a significant enrichment of the oxidative phosphorylation pathway in the excluded and infiltrated tumors (adjusted p-value=0.04, FIGS. 4A, 4C, and 16). Components of this pathway including subunits of NADH dehydrogenase (NDUF variant genes), succinate dehydrogenase (SDHC, SDHA, SDHB), cytochrome c oxidase (COX variant genes) and V-type ATPase showed a continuum of expression levels, from low (on average) in tumor cells of desert tumors, to intermediate in excluded tumors, to high expression in infiltrated tumors (FIGS. 4C and 16).

### Example 3. Distinct States of CD8⁺ T Cells Characterize Immune Infiltrated and Excluded Tumors Immune Phenotypes

### 3.1. Multiplex Immunofluorescence (IF)

4 μM tissue sections from 17 ovarian cancer samples procured from Cureline, Inc were subject to multiplex IF assays performed on a VENTANA BenchMark Ultra automated staining instrument (Ventana Medical Systems). The detailed description of epitope retrieval from FFPE tissue sections, antibody titration, incubation and image acquisition were previously described. Zhang, W. et al., Lab. Invest., 97:873-885 (2017). In brief, for each target, the corresponding 1° antibody (10 Ab) (human CD3 (#ab135372, Abcam), GZMB (#14-8889-80, ThermoFisher Scientific), pan-cytokeratin (#760-2595, Roche), PD-1 (#ab52587, Abcam) or PD-L1 (#790-4905, Roche)) was incubated on the slide, followed with a horseradish peroxidase (HRP) conjugated 2° Ab (GaRt-HRP (#760-4457) for GZMB, GaMs-HRP (#760-7060) for PD1 and pan-cytokeratin, and GaRb-HRP (#760-7058) for CD3 and PDL1); the target was then detected with a tyramide-conjugated fluorophore (TSA-FL). The next target detection followed the same scheme, and so on. To prevent potential cross-reaction of same species 1° antibodies, a heating step was introduced to deactivate the 1° Ab & 2° Ab complex before detecting the next target. Slides were then counterstained with 4′,6-diamidino-2-phenylindole (DAPI) (Roche Tissue Diagnostics, Cat #760-4196). Slides were cover slipped using micro cover glass, 24×50 mm no. 1.5 (VWR, Cat #: 48393241) and a ProLong™ Diamond Antifade Mountant with DAPI (ThermoFisher Scientific, Cat #: P36962). The fluorescent image acquisition was performed in a ZEISS Axio Scan.Z1 (Oberkochen, Germany). Image analysis on counting of cells with either uniquely stained or concurrently stained markers within regions of interest (ROI), i.e. tumor, panCK+ epitumor or stroma regions was performed on scanned images as previously described. Racolta, A. et al., J. Immunotherapy Cancer, 7:282, doi:10.1186/s40425-019-0763-1 (2019). These ROIs were computed by the DP algorithms based on pathologists' manual annotation of tumor lesion and invasive front of the tumor on the images. Necrotic areas were manually excluded. For each whole slide, individual fields of view (FOV) are tiled and processed. Digital pathology (DP) algorithm is used to identify phenotypes/regions of interest that are detected by the markers. In detail, the algorithm includes the following steps: (1) Preprocessing: preprocessing was applied to remove a variety of fluorescence artifacts in FOVs. (2) Cell detection: the radial symmetry algorithm was used to detect and vote for the center of the cells. (3) Feature extraction: morphology, appearance, intensity, gradient, and direction features were extracted. (4) Cell classification: different machine learning classifiers such as support vector machine, random forest, and logistic regression algorithms were used. Accuracy of each classification was subsequently assessed. A classifier with the best accuracy was used to classify the cells. (5) Epitumor area and stroma region segmentation: a method combining region growing and adaptive thresholding was used to segment epitumor and stroma area. After identifying the phenotypes/regions of interest, the DP algorithm reports statistical metrics that characterize the density of objects and their spatial interrelationships in automatically computed ROIs. Different categories of readout analysis were reported: (1) ROI areas; (2) counts of phenotypes within different ROIs; (3) counts of cells with specific characteristics; and (4) counts of phenotypes at different distances from ROIs. Two samples were excluded from the downstream analysis due to lack of any triple positive cells.

### 3.2. Clinical Datasets

Three ovarian cancer clinical datasets were used in this study to validate our scRNAseq findings: i) ICON7 collection (n=351), clinical trial registration: NCT00483782; ii) ROSiA collection (n=308), clinical trial registration: NCT01239732; and iii) TCGA collection (n=412). Bell, D. et al., Nature Publishing Group, 474:609-615 (2011). Study protocols were compliant with good clinical practice guidelines and the Declaration of Helsinki. Ethics approval was obtained in all participating countries and where required in all participating centres. All patients provided written informed consent.

### 3.3. RNAsitu Hybridization (ISH)

RNA in situ hybridization assays for the dual detection of CD8A and GZMB or CD8A and GZMK in 5 μm FFPE ovarian tumor tissue sections were performed using the RNAscope® 2.5 LS Duplex Reagent Kit (ACD, Cat #322440) with the RNAscope® 2.5 LS Green Accessory Pack (ACD, Cat #322550) on the BOND RX automated stainer (Leica Biosystems) according to the manufacturer's instructions (Advanced Cell Diagnostics, a Bio-Techne brand, Newark, CA). Tissue RNA quality was assessed using positive control probes Hs-PPIB (ACD, Cat #313908) for human cyclophilin B (PPIB) and Hs-POLR2A (ACD, Cat #310458-C2) for human RNA polymerase subunit IIA (POLR2A) and negative control probe dapB (ACD, Cat #320758) for bacterial dihydrodipicolinate reductase (dapB). Only samples demonstrating acceptable RNA quality as defined by the presence of an average of ≥4 dots per cell with the positive control probe staining and an average of <1 dot per 10 cells with the negative control probe staining were further analyzed with the target probes for CD8A (ACD, Cat #560393, NM_001768.6, 971-2342 nt), GZMK (ACD, Cat #475903-C2, NM_002104.2, 25-1025 nt) and GZMB (ACD, Cat #445973-C2, NM_004131.4, 3-912 nt). FFPE HeLa cell control slides were tested in parallel for POLR2A and dapB as run controls along with the ovarian cancer FFPE tissue slides. The resulting slides were scanned with a 3DHistech Pannoramic™ SCAN II digital slide scanner (Thermo Fisher Scientific) using a 40× objective. Scanned images were analyzed for CD8A, GZMB and GZMK staining in the tumor and tumor associated stroma regions using the HALO® image analysis software (Indica Labs). RNAscope signals were counted and binned into 5 categories based on the number of dots per cell (bin 0=0 dot/cell, bin 1=1-3 dots/cell, bin 2=4-9 dots/cell, bin 3=10-15 dots/cell, and bin 4=>15 dots/cell with >10% of dots in clusters). A composite H-score was calculated to combine the signal level and the percentage of cells in each bin as follows: H-Score=(0×% cells in bin 0)+(1×% cells in bin 1)+(2×% cells in bin 2)+(3×% cells in bin 3)+(4×% cells in bin 4). The H-scores ranged from 0 to 400. H-scores for tumor and stroma regions were scored separately.

### 3.4. Preparation of Bulk RNAseq Libraries, and Sequencing

A fresh tissue sample was mechanically dissociated with RLT buffer (#79216, Qiagen), followed by RNA extraction (#74136, Qiagen). Libraries were generated using TruSeq (#20020595, Illumina) following the manufacturer's instructions, pooled and sequenced on an Illumina NextSeq500 with the High output kit v2 (#20024907, Illumina).

### 3.5. Batch Effect Correction and Diffusion Pseudotime Analysis

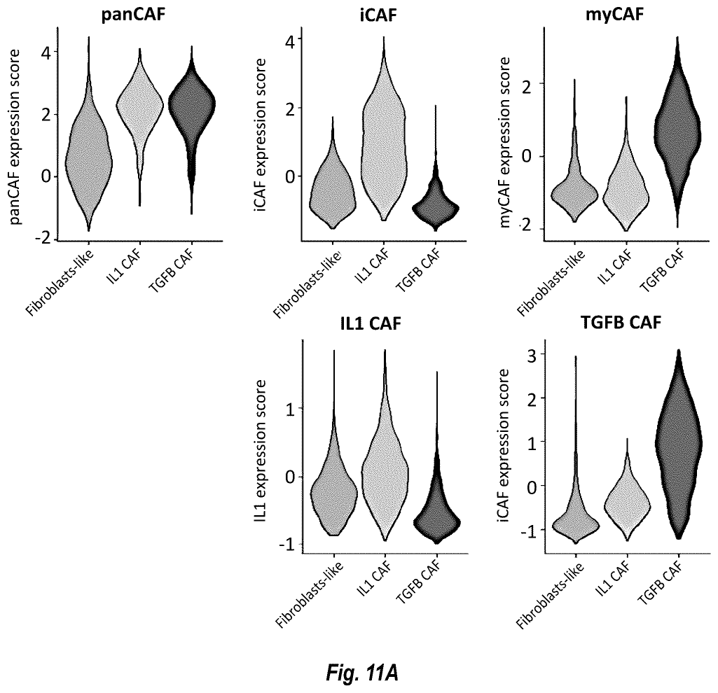

Since the analysis of the fibroblast, T cell and myeloid populations revealed patient-driven clustering, we corrected for the patient effects (herein batch effects) using the batch-balanced k-nearest neighbour correction (bbknn) method. Polanski, K. et al., Bioinformatics. 36:964-965 (2020). To this end, we imported the python modules scanpy v1.4.3 including anndata v0.6.21, numpy v1.17.0, bbknn v1.3.9 into R using reticulate v1.12. In brief, bbknn was applied to the top principal components as computed by seurat and determined by the elbow plot, clusters were identified using scanpy and all results transferred back to seurat. To validate that bbknn only corrected for technical differences and not for biological differences, the resulting clusters and their markers were manually compared to the results of a single library cluster analysis (FIGS. 11A and 11B).

### 3.6 Diffusion Pseudotime Analysis

The diffusion pseudotime analysis was performed through the scanpy diffusionmap function on the bbknn− corrected anndata object and transferred back to seurat.

### 3.7. Identification of Subpopulations and Subcellular Functions

The fibroblast cluster identities were determined by calculating gene signature scores using seurat AddModuleScore of previously identified fibroblast phenotypes: iCAF, myCAF, and IL1-driven and TGFB-driven CAF. Dominguez, C. X et al., Cancer Discovery, 10:232-253 (2020); Elyada, E. et al., Cancer Discovery, 9:1102-1123 (2019).

Established myeloid and T cell type clusters were annotated using the cluster mean expression of the following gene markers: 1) dendritic cells: CD1C, CLEC10A, CSF2RA, CCL19, CCR7, 2) plasmacytoid dendritic cells: LILRA4, 3) proliferative cells: MKI67, 4) Tgd and NK cells: TRDC, NCAM1, 5) CD8 T cells: CD8A, CD8B, 6) CD4 T cells: CD4, CD40LG. A cluster of cells with high expression of proliferative genes such as MKI67, PCNA and BIRC5 was observed in every cell type analyzed, as is often the case in single-cell analyses. These proliferative cell clusters typically represented a mixture of different subpopulations which cannot be separated due to the dominant cell cycle gene expression program, and we removed these uninformative clusters from all downstream analyses. Cutoffs for the annotation by mean expression were determined by manual inspection of the clusters and the gene expression distributions. The subpopulation gene markers of the T cell, myeloid cell and fibroblast populations as plotted on the gene marker heatmaps and were identified by testing for significant differential expression in a subpopulation against all other cells using the Wilcoxon test.

### 3.8. Bulk RNA Sequencing Data Processing and Immune Phenotype Prediction

Raw data processing of the KIYATEC, ICON7, ROSiA and TCGA datasets was performed as described previously. Desbois, M. et al., Cancer Res., 79:463 (2019). In brief, raw counts were filtered for lowly expressed genes for which the counts per million (CPM) were smaller than 0.25 in at least 10% of samples. CPM was calculated with the cpm function in the edgeR package. Based on the raw counts of the filtered gene expression matrix, size factors were calculated using CalcNormFactors (edgeR package) and used for subsequent voom-limma differential expression analysis. The immune phenotype of each sample was predicted using our previously built gene expression based classifier applied to housekeeping gene normalized data as described previously. (Desbois, M et al., Nature Communications, 11:5583, doi:10.1038/s41467-020-19408-2 (2020).

### 3.9. Bulk RNA Sequencing Data Deconvolution and Signature Scoring

To estimate the proportion of CD8+ GZMB and CD8+ GZMK positive cells, the expression level of GZMB or GZMK over the CD8+ CIBERSORT signature score was calculated for each sample. Newman, A. M. et al., Nat. Meth., 12:453-457 (2015). In detail, the log and voom transformed data was used to calculate a z-score of the CIBERSORT_LM22_T_cells_CD8 gene set using scoreSingleSamples from the multiGSEA package. GZMB and GZMK were excluded from the gene set. Then, the CD8+ signature score was subtracted from the log and voom transformed GZMB or GZMK expression.

### 3.10. Survival Analysis

Analysis of the association of CD8+ GZMK and GZMB T cells with progression-free survival in the ICON7 chemotherapy arm was conducted using the survival package.

### 3.11. Results

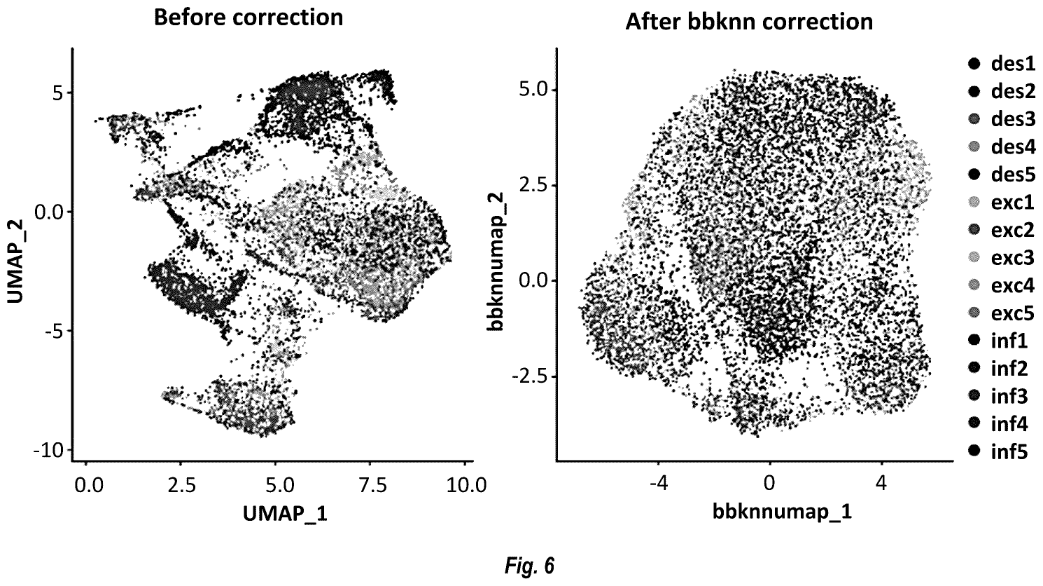

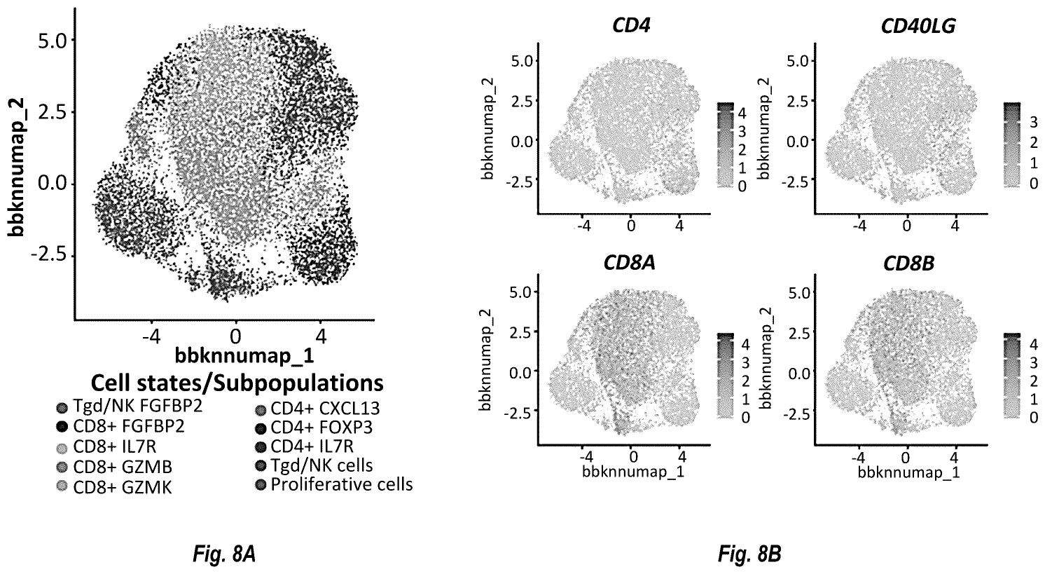

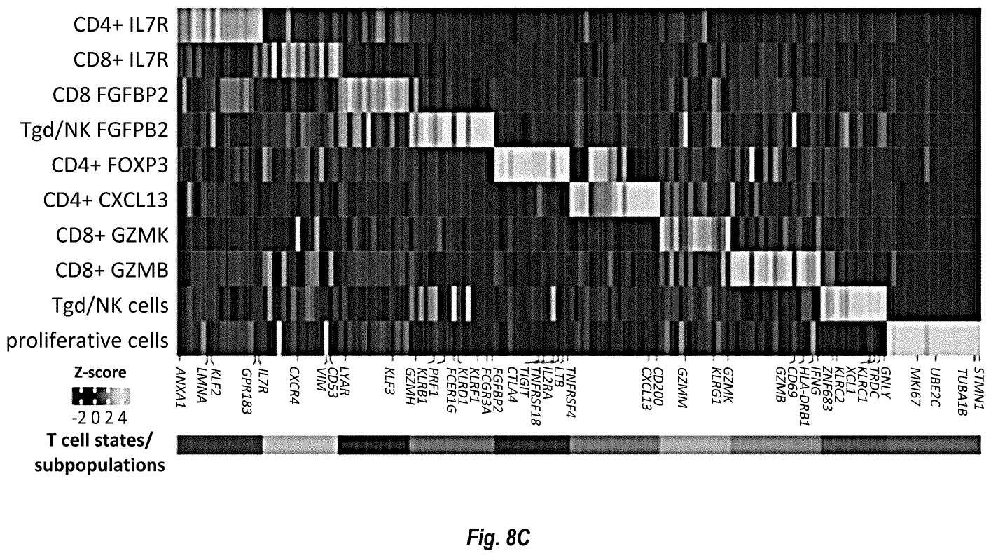

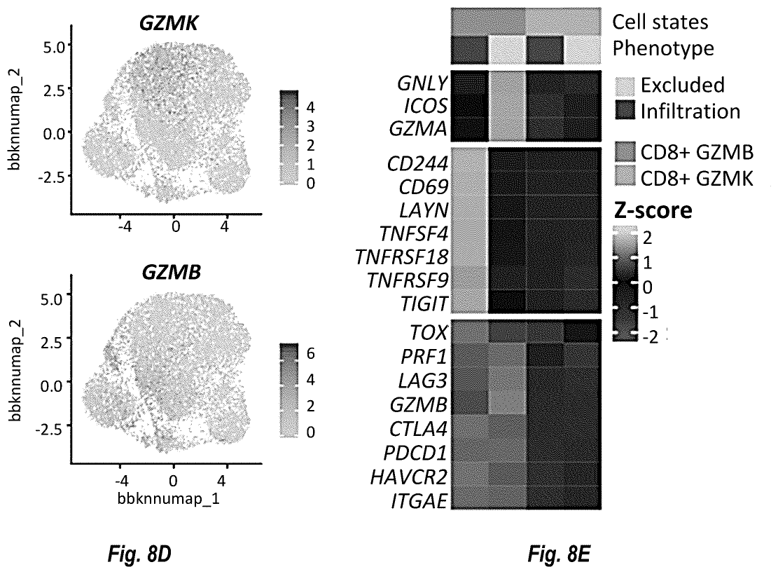

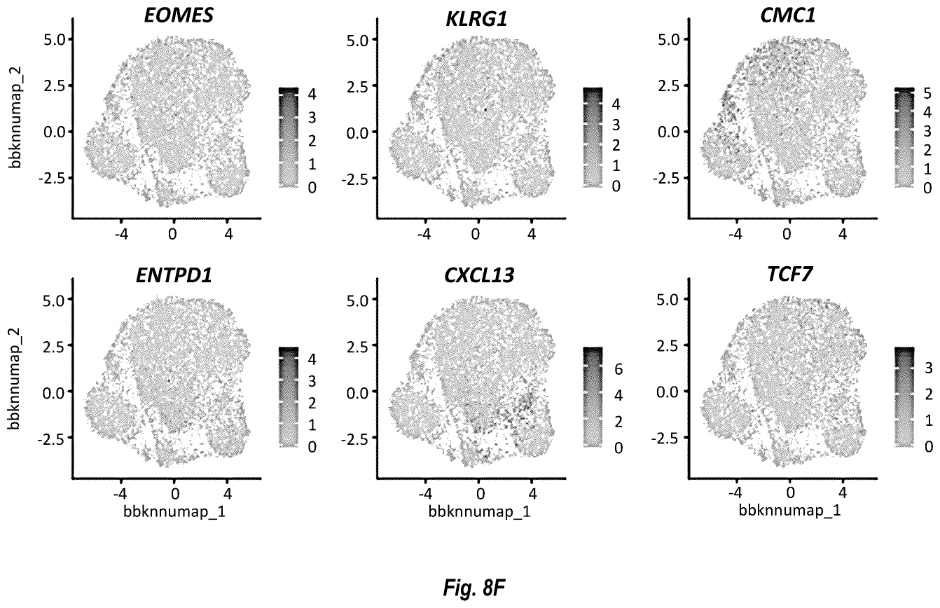

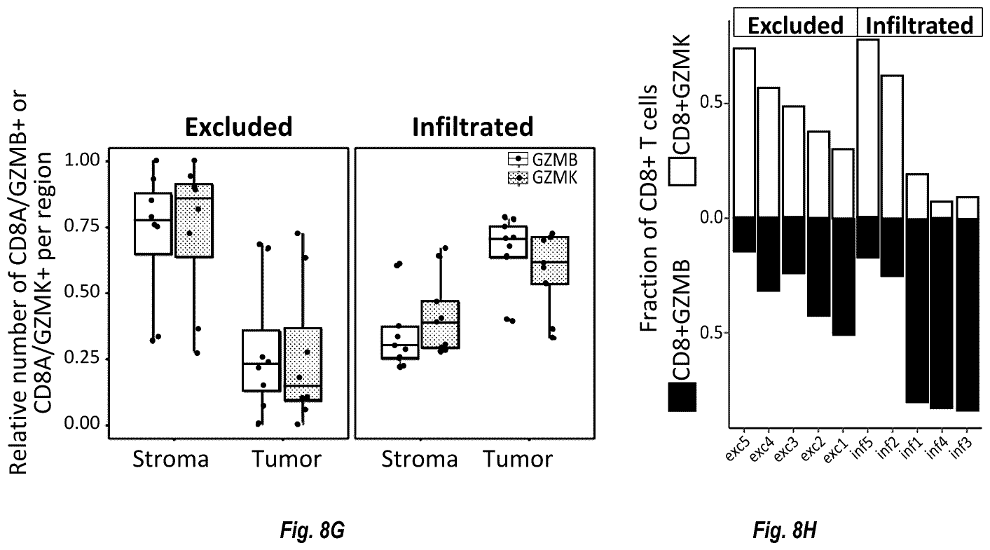

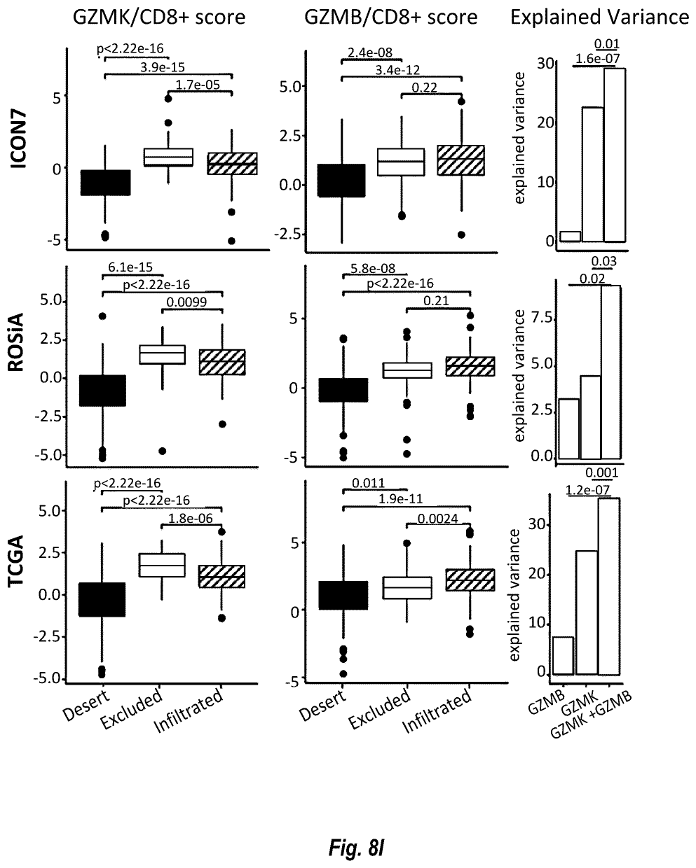

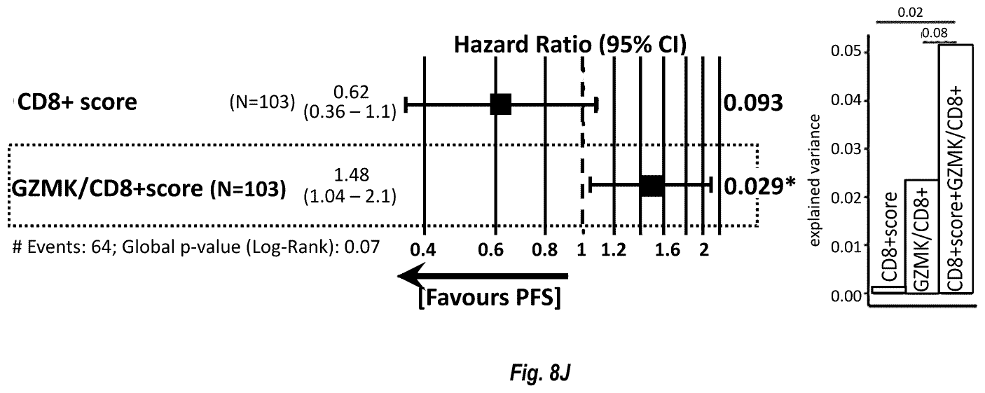

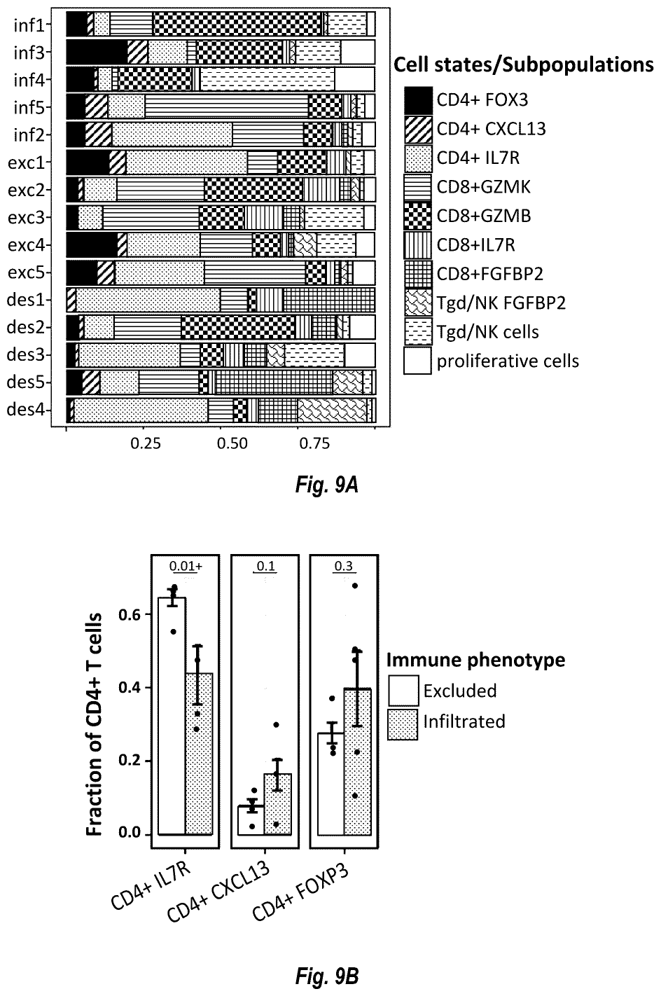

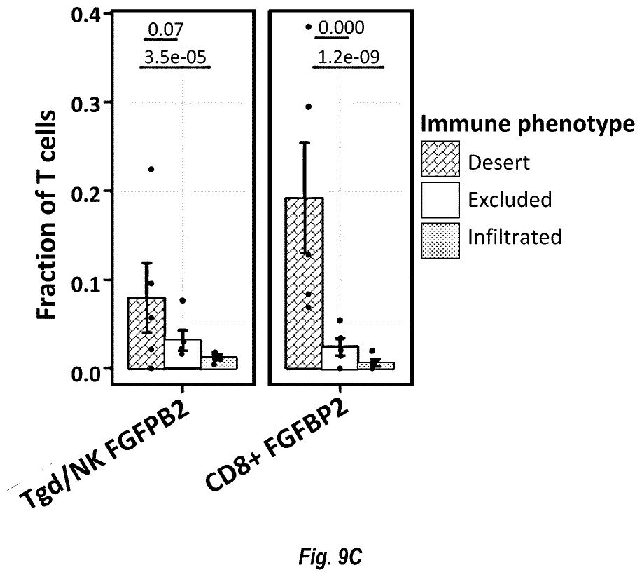

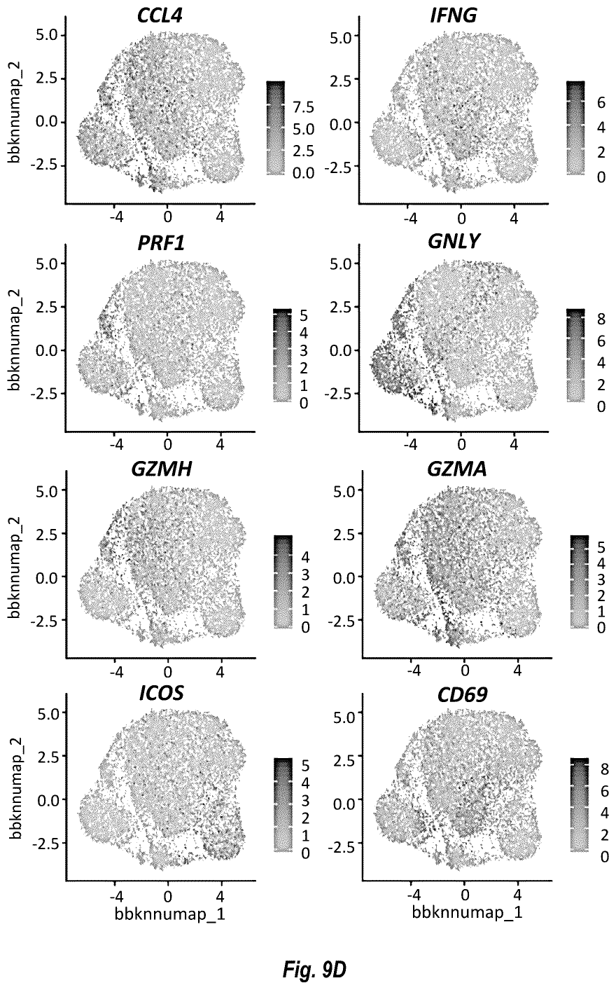

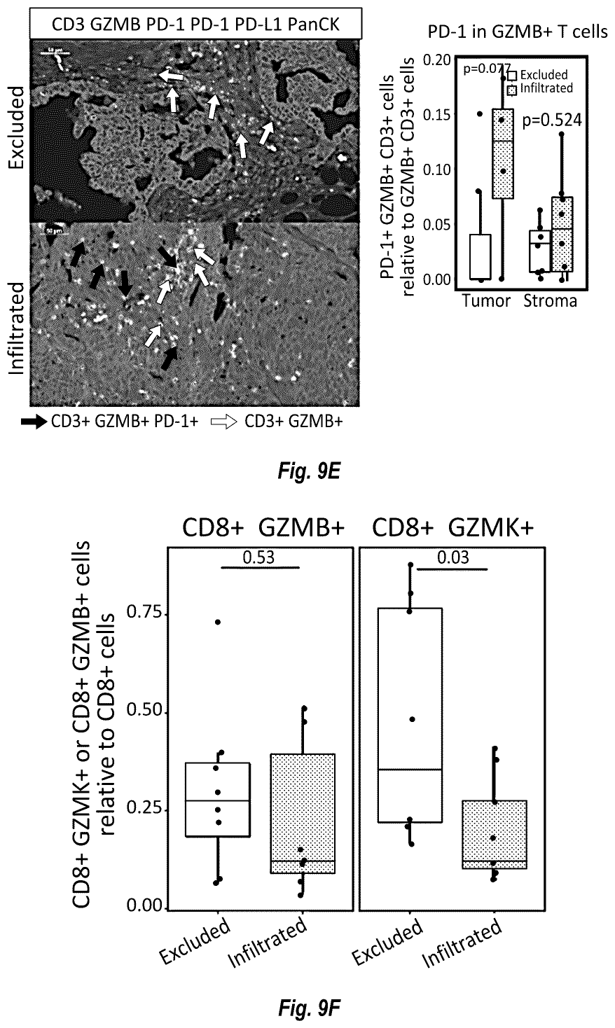

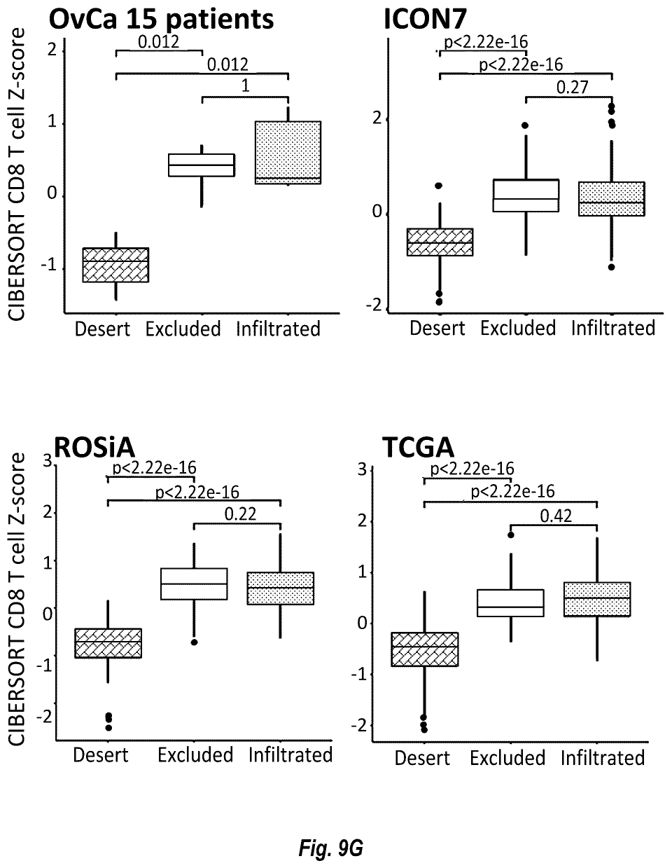

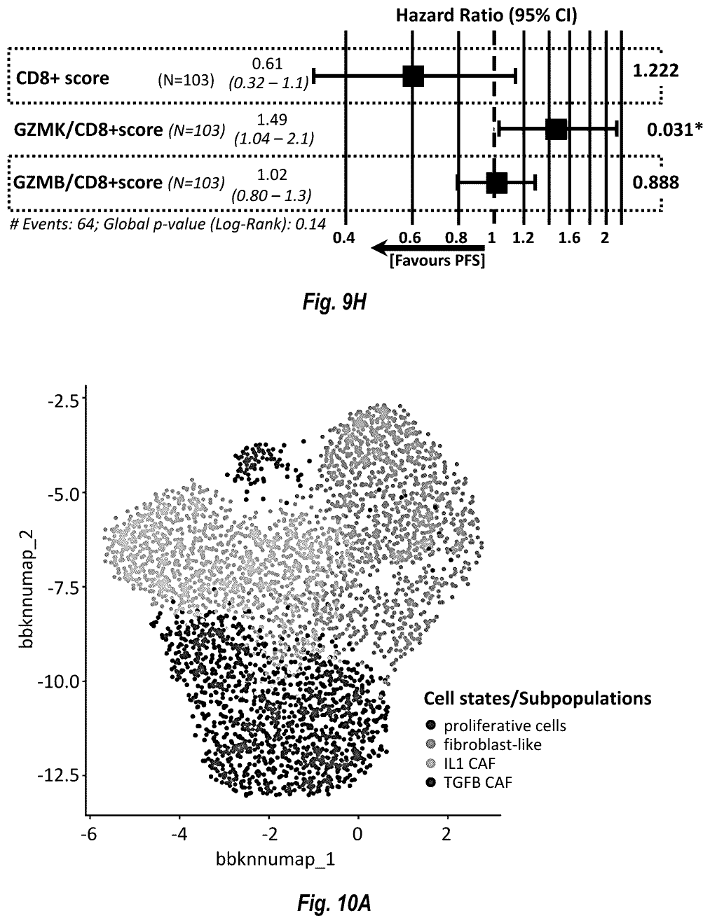

To dissect the composition of the tumor microenvironment for each immune phenotype, we investigated each cell type separately (i.e. T cells and myeloid cells) and defined cell subpopulations and cellular functions. Cluster analysis on the batch-corrected uMAP representation of all T cells revealed a separation of CD8+ from CD4+ T cells (FIGS. 8A, 8B, and 6A). In addition, we identified two clusters expressing the T gamma delta receptor gene, TRDC, and the NK cell marker, NCAM1. Because NK cells and T gamma delta (Tgd) cells are transcriptionally similar, we were not able to differentiate these two cell types. Within the CD4+ T cells, we distinguished three distinct functional and phenotypic states according to their gene expression markers, being regulatory CD4+ T cells (FOXP3, CTLA4, IL2RA), activated CD4+ T cells (CXCL13, CD200, ICOS) and resting CD4+ T cells (IL7R, GPR183, LMNA, ANXA1) (FIGS. 8A and 8C). The resting CD4+IL7R T cells were significantly enriched in excluded tumors compared to infiltrated tumors (p-value=0.01), while infiltrated tumors tended to have more activated and regulatory CD4+ T cells (p-value=0.1 and 0.3, FIGS. 9A and 9B). We identified four CD8+ T cell states and annotated them based on their featured marker and using the CD8+ T cell nomenclature reported by van der Leun and colleagues: i) CD8+ FGFBP2, ii) CD8+IL7R, iii) CD8+ GZMB and iv) CD8+ GZMK (FIGS. 8A and 8C). van der Leun, A. M. et al., Nat. Rev. Cancer, 20:218-232 (2020). CD8+ FGFBP2 cells have been previously reported as cytotoxic effector CD8+ T cells. Li, T. et al., Cell, 176(4):775-789.e18 (2019). Interestingly, a distinct cluster of the Tgd/NK cells shared markers with the CD8+ FGFBP2 population: FGFBP2, PRF1, GZMB, KLRG1 and GLNY (FIGS. 8A and 8C). Both CD8+ FGFBP2 and Tgd/NK FGFBP2 populations were significantly enriched in the desert tumors and almost absent in the infiltrated tumors (p=0.003 and 0.07 in excluded, p=1.2e-09 and 3.5e-05 in infiltrated, FIGS. 9a and 9c), however, the absolute cell numbers were relatively small (data not shown). The CD8+IL7R cells express IL7R and GPR183 (FIG. 8C), with a transcriptional profile resembling central memory CD8+ T cells previously observed in NSCLC and CRC (Guo, X. et al., Nature Medicine, 24:978-985 (2018); Zhang, L. et al., Nature, 564:268-272 (2018)) and regrouped under naïve-like cells based on the nomenclature proposed by Van der Leun, A. M. et al., Nat Rev Cancer, 20, 218-232 (2020).

We next focused on the two largest CD8+ T cell clusters, CD8+ GZMB and CD8+ GZMK T cells (FIGS. 8C and 8D), and further characterized their potential functional states and spatial distribution in detail. Although both GZMB and GZMK T cell populations have been previously reported in scRNAseq studies, their functions were previously poorly understood. Guo, X. et al., Nature Medicine, 24:978-985 (2018); Wu, T. D. et al., Nature Publishing Group, 579:274-278 (2020); Yost, K. E. et al., Nature Medicine, 25:1251-1259, doi:10.1038/s41591-019-0522-3 (2019); Zhang, L. et al., Nature, 564:268-272 (2018); Zhang, Q. et al., Cell, 179:829-845.e20 (2019). Our data revealed that both populations expressed markers that are suggestive of an activated phenotype (ICOS, CD69), and both demonstrated effector functions by expression of other granzymes (GZMA, GMZH), interferon gamma (IFNG), perforin (PRF1), granulysin (GNLY) and CCL4 (FIG. 9D). Yet, they differed markedly in their activation and exhaustion-like status. The CD8+ GZMB population displayed a profoundly activated and exhausted-like phenotype as suggested by expression of CTLA4, TOX, LAG3 and PDCD1 (FIG. 8E). In addition, the CD8+ GZMB population was marked by the expression of ENTPD1 (CD39) and CXCL13 (FIG. 8F). CD39 and CXCL13 have been previously identified as potential markers of tumor-reactive CD8+ T cells and late dysfunctional T cells, respectively. Simoni, Y. et al., Nature, 557:575-579 (2018); van der Leun, A. M. et al., Nat. Rev. Cancer, 20:218-232 (2020).

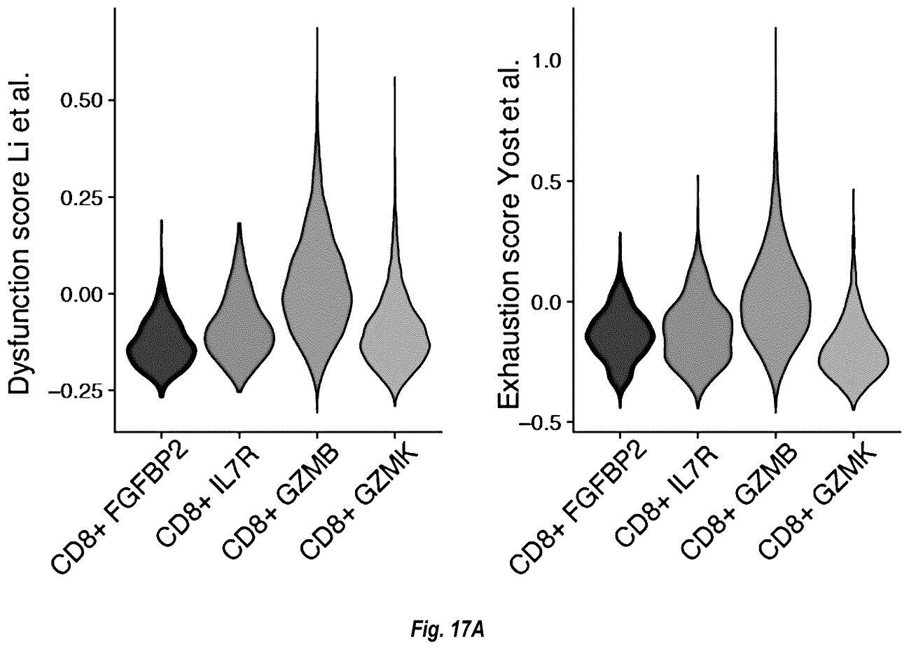

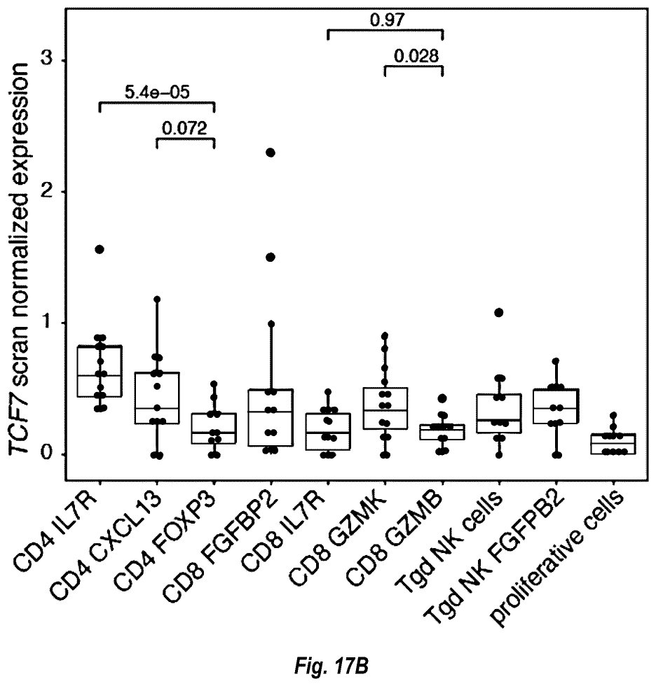

Interestingly, the activation/exhaustion state of CD8+ GZMB T cells was significantly greater in infiltrated compared to excluded tumors, with higher expression of activation and exhaustion markers such as LAYN, CD69 and TNFRSF9 (FIG. 8E). Applying previously developed dysfunction score (Li H. et al., Cell, 176, 775-789 e718 (2019)) and core T cell exhaustion signature (Yost, K. E. et al., Nature Medicine, 25, 1251-1259 (2019)) to our dataset also confirmed that the CD8+ GZMB T cells showed the highest degree of dysfunctionality and exhaustion (FIG. 17). To validate the activation state of CD8+ GZMB T cells in infiltrated versus excluded tumors at the protein level, we subjected 17 ovarian cancer tissues (9 infiltrated, 8 excluded) from an independent validation collection to multiplex immunofluorescence for cytokeratins (PanCK), CD3, PD-1 and GZMB. Among all GZMB+ T cells (CD3/GZMB double positive), the fraction of GZMB+ T cells positive for PD-1 was significantly higher in the infiltrated tumors compared to the excluded tumors in the tumor epithelium (p-value=0.077) (FIG. 9E) supporting the higher activation state of GZMB+ T cells in infiltrated tumors specifically of the GZMB+ T cells in the tumor area.

In contrast, CD8+ GZMK cells have been previously described as potential effector memory T cells or pre-dysfunctional T cells. Guo, X. et al., Nature Medicine, 24:978-985 (2018); Li, H. et al., Cell, 176:77-789 (2019); doi:10.1016/j.cell.2018.11.043; Wu, T. D. et al., Nature Publishing Group, 579:274-278 (2020); Zhang, L. et al., Nature, 564:268-272 (2018); Zheng, C. et al., Cell, 169:1342-1356.e16 (2017). We found a similar marker profile expression in the CD8+ GZMK cell population including EOMES, KLRG1 and CMC1 (FIG. 8F) as reported in these studies. Together, these observations suggest a more advanced dysfunctional state of CD8+ GZMB cells that might be linked to their tumor reactive potential while CD8+ GZMK T cells represent pre-dysfunctional effector memory cells.

Given the different dysfunctional states of CD8+ GZMB and CD8+ GZMK T cells, we explored whether the spatial distribution of these T cell subsets in stromal vs. tumor epithelium contributes to the difference in their functional state. We subjected the same 17 ovarian cancer samples from the validation collection to an RNAscope assay for in situ co-hybridization of GZMK/CD8A and GZMB/CD8A as well as localization within the stromal vs. tumor area through H&E staining. Both CD8A/GZMK and CD8A/GZMB double positive T cells accumulated in peritumoral stroma of excluded tumors, and in tumor epithelium of infiltrated tumors (FIG. 8G). From this observation we concluded that both CD8A/GZMK and CD8A/GZMB T cells have the capacity to infiltrate the tumor epithelium at least in infiltrated tumors and that therefore the proximity to the tumor epithelium cannot explain the differences in the dysfunctional states of the GZMB+ and GZMK+ populations.

We next asked whether the dysfunctional CD8+ GZMB and pre-dysfunctional CD8+ GZMK T cells showed a different prevalence in excluded compared to infiltrated tumors based on our scRNAseq analysis: 3 out of 5 infiltrated tumors showed a lower CD8+ GZMK fraction compared to all excluded tumors and the reverse for the CD8+ GZMB subpopulation (FIGS. 8H and 9A). We hypothesized that there is an association of the CD8+ GZMK pre-dysfunctional subpopulation with the excluded tumors and CD8+ GZMB dysfunctional subpopulation with infiltrated tumors. To validate this hypothesis, we used an RNAscope assay and a bulk RNAseq deconvolution approach. Our RNAscope assay results showed a significantly higher proportion of CD8A/GZMK double positive cells in excluded samples compared to infiltrated samples (p=0.03), but no significant difference in the proportion of CD8A/GZMB double positive cells (FIG. 9F).

In addition to in situ validation, we validated our scRNAseq findings in larger studies by investigating bulk RNAseq data from three ovarian cancer cohorts: TCGA (n=412), (Cancer Genome Atlas Research, 2011), the ICON7 clinical trial collection (n=351), and the ROSiA clinical trial cohort (n=308). Bell, D. et al., Nature Publishing Group, 474:609-615 (2011); Oza, A. M. et al., Int. J. Gynecol. Cancer, 27:50-58 (2017); Perren, T. J. et al., N. Engl. J. Med., 365:2484-2496 (2011). We first predicted the tumor immune phenotype of these samples with our previously established classifier. Desbois, M. et al., Cancer Res., 79:463 (2019). Deconvolving bulk RNAseq data using the CIBERSORT CD8 signature confirmed a higher CD8⁺ T cell content in excluded and infiltrated tumors in all three cohorts, similar to what we observed in the 15 ovarian cancer samples used in our study (FIG. 9G). To validate the immune phenotype-specific enrichment of the CD8+ GZMK versus GZMB T cell populations, we used the ratio of GZMK or GZMB expression over the CD8+ T cell signature score for each sample as a surrogate for the fraction of each population among CD8+ T cells. Consistent with our model, in all three clinical cohorts, excluded tumors showed a significantly higher GZMK/CD8+ surrogate ratio compared to infiltrated tumors (FIG. 8I). Similarly, the GZMB/CD8+ ratio was higher in infiltrated than excluded tumors, though statistical significance for this difference was only achieved in the larger TCGA dataset. Furthermore, combining the GZMB/CD8+ and GZMK/CD8+ ratios in a logistic regression model predicting infiltrated vs. excluded phenotype significantly increased the explained variance over either single-ratio model in all three cohorts (FIG. 8I), highlighting that the fraction of CD8+ GZMK and GZMB cell populations are both associated with phenotype, and thus supporting that these tumor immune phenotypes show important qualitative and quantitative differences in their CD8+ T cell functional states.

In our previous study we observed reduced survival of ovarian cancer patients on chemotherapy when their tumors exhibited an excluded phenotype compared with an infiltrated or desert phenotype. Desbois, M. et al., Cancer Res., 79:463 (2019). Because of the differential association of CD8+ GZMB and GZMK cells with infiltrated and excluded tumors, we asked whether the CD8+ T cell state also associates with clinical outcome under chemotherapy treatment. In a cohort of 103 patients from the chemo-treatment arm of the ICON7 clinical trial, we observed that CD8+ T cell quantity was weakly associated with longer progression-free survival (PFS) (p=0.093, FIG. 8J), consistent with previous findings indicating a good prognostic effect for CD8 T cell infiltration in ovarian cancer. Hwang, W. T. et al., Gynecol. Oncol., 124:192-198 (2012); Lo, C. S. et al., Clinical Cancer Research, 23:925-934 (2017); Zhang, L. et al., N. Engl. J. Med., 348:203-213 (2003). Interestingly, while the GZMB/CD8+ ratio was not associated with clinical outcome in this cohort, the GZMK/CD8+ ratio was significantly associated with shorter PFS, particularly when combined with the overall CD8+ T cell score in a multivariate model (p=0.029, FIGS. 8J and 9H). Thus, the relative proportion of CD8+ T cells that are GZMK positive is associated with reduced survival, and this ratio provides more information about patient outcome than overall CD8+ T cell content.

In summary, activated CD4+ and CD8+ GZMB T cells are enriched in infiltrated tumors, whereas resting CD4+ and CD8+ GZMK T cells are enriched in excluded tumors, and both CD8+ T cell types can be found in the tumor epithelium. Characterization of the CD8+ T cell populations revealed a more exhausted cytotoxic effector function phenotype for CD8+ GZMB T cells, while markers of effector memory T cells characterize the CD8+ GZMK T cells. Finally, a higher proportion of CD8+ GZMK T cells is associated with worse outcome under chemotherapy in ovarian cancer.

### Example 4. Fibroblast Phenotypes Associate with the Localization of T Cells

### 4.1. Results

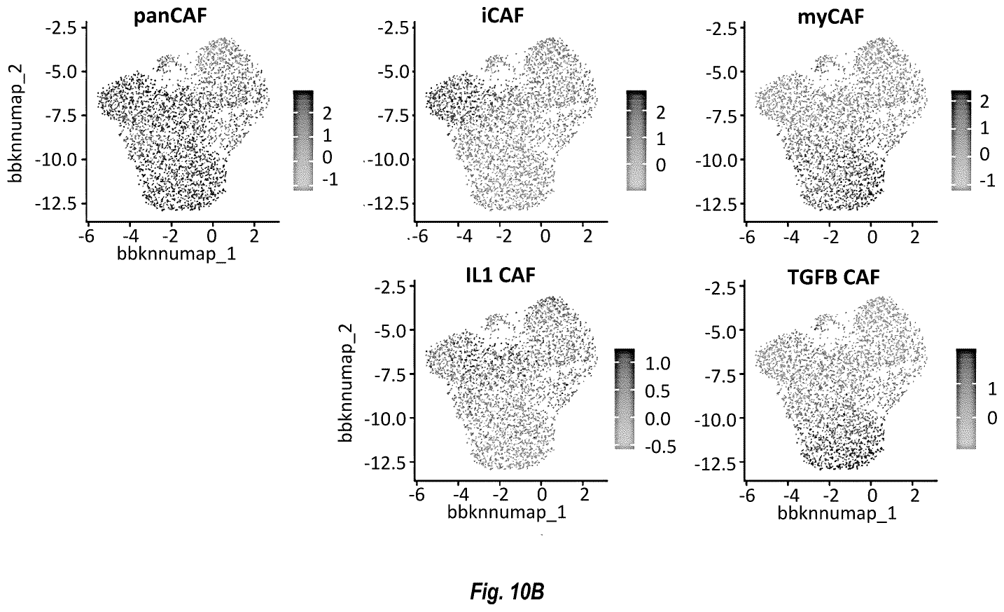

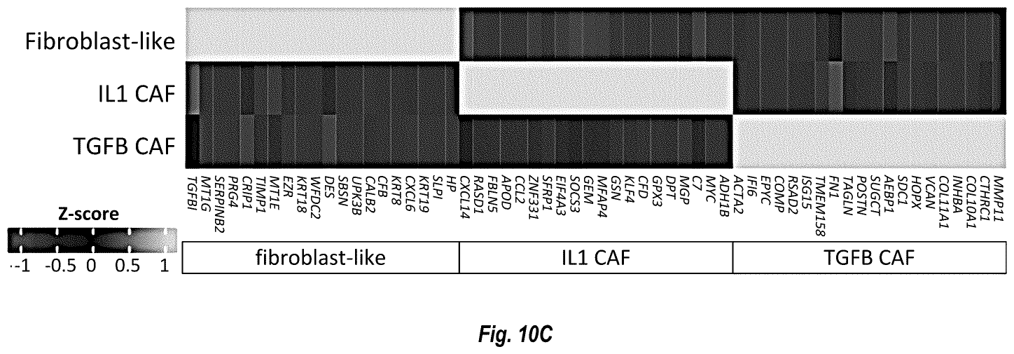

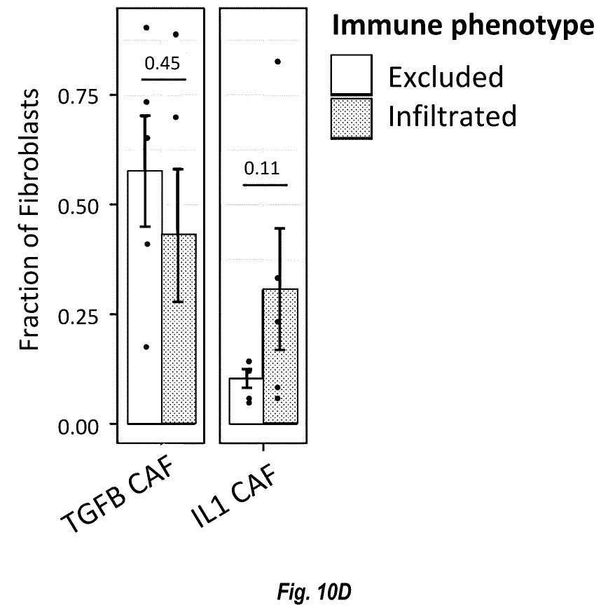

Recent studies have revealed distinct subsets of cancer associated fibroblasts (CAFs) in several tumor types. Avery, D. et al., Matrix Biol., 67:90-106 (2018); Costa, A. et al., Cancer Cell, 33:463-479.e10 (2018); Dominguez, C. X. et al., Cancer Discovery, 10:232-253 (2020); Ohlund, D. et al., J. Exp. Med., 214:579-596 (2017). However, their association with tumor immune phenotypes is poorly understood. We identified three main clusters of fibroblasts (FIG. 10A). Two out of the three fibroblast clusters showed a high expression of the panCAF signature (derived from Elyada, E. et al., Cancer Discovery, 9:1102-1123 (2019)), indicating the presence of CAFs (FIGS. 10B and 11A). Previously, CAFs have been described as either inflammatory CAFs (iCAF) and myofibroblasts (myCAF) or as IL1-activated (IL1 CAF) and TGFB-activated CAFs (TGFB CAF). Dominguez, C. X. et al., Cancer Discovery, 10:232-253 (2020); Elyada, E. et al., Cancer Discovery, 9:1102-1123 (2019). Of the two clusters with strongest panCAF signal, one cluster displayed high iCAF and IL1_CAF signature expression, while the other cluster showed high myCAF and TGFB_CAF expression (FIGS. 10B and 11A). We therefore annotated these clusters as IL1 CAF and TGFB CAF, respectively.

To gain further insights into the functions of these distinct CAF populations, we interrogated the gene expression profiles differentiating TGFB CAFs and IL1 CAFs. The top 20 markers of the TGFB CAF population include genes previously associated with TGFβ-induced reactive stroma: periostin (POSTN), smooth muscle actin (ACTA2), cartilage oligomeric matrix protein (COMP) as well as collagen subunits (COL10A1, COL11A1), matrix metalloproteinases (MMP11), transgelin (TAGLN) and fibronectin (FN1) (FIG. 10C). Ryner, L. et al., Clinical Cancer Research, 51:2941-2951 (2015). On the other hand, markers characterizing the IL1 CAF population included cytokine/chemokine signaling features such as CXCL14, CCL2, and suppressor of cytokine signaling 3 (SOCS3). Dominguez, C. X. et al., Cancer Discovery, 10:232-253 (2020); Elyada, E. et al., Cancer Discovery, 9:1102-1123 (2019); Higashino, N. et al., Lab. Invest., 99:777-7992 (2019). We previously identified an association between the TGFβ-mediated reactive stroma and the T cell excluded immune phenotype in ovarian cancer. Desbois, M. et al., Cancer Res., 79:463 (2019). Hence, we analyzed whether the distinct fibroblast subpopulations might be linked to different tumor immune phenotypes: IL1 CAFs show a trend to be enriched in infiltrated tumors (p=0.11), however, the association of TGFB CAFs with the excluded tumors is not significant, (FIGS. 10D and 11B). Although these TGFB CAFs express reactive stroma genes creating a dense matrix that could contribute to the exclusion of tumor cells and represent ≥40% of fibroblast cells in 4 out of 5 excluded tumors and only 2 out of 5 infiltrated tumors, additional investigations are warranted including the distribution of these cells in the TME to further dissect their association with excluded tumors.

The third cluster of fibroblasts showed appreciable but reduced expression of the panCAF signature (FIG. 11A). We annotated this cluster as a fibroblast-like population (FIG. 11B). Of note, these fibroblast-like cells were found almost exclusively in one desert (des3) and one excluded tumor (exc4) (FIG. 11i). The top markers for this cluster include genes that have been associated with epithelial cells (SLPI, KRT19, KRT8, KRT18, WFDC2). Shih, A. J. et al., PLoS ONE, 13: e0206785 (2018). As such, it is possible that this cluster represents tumor cells that have undergone EMT. Additional investigations are needed to better understand the nature of these cells.

### Example 5. Phenotypically and Functionally Diverse Subsets of Myeloid Cells Linked to Different Tumor Immune Phenotypes

### 5.1. Results

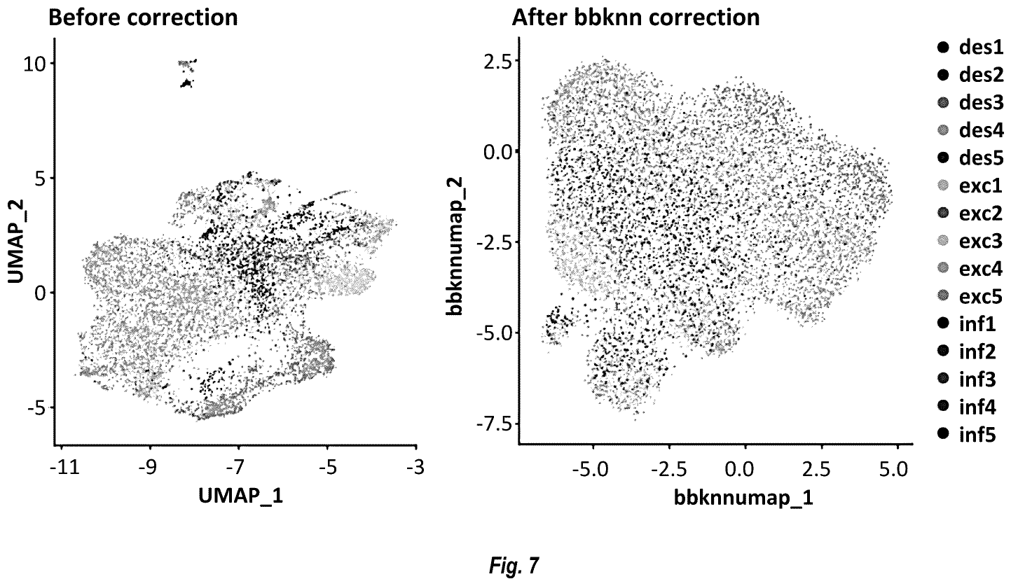

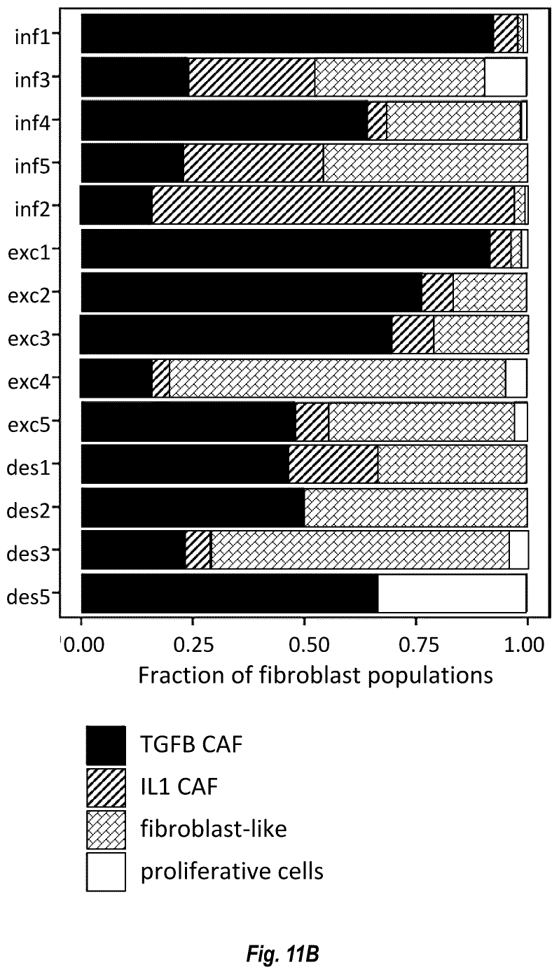

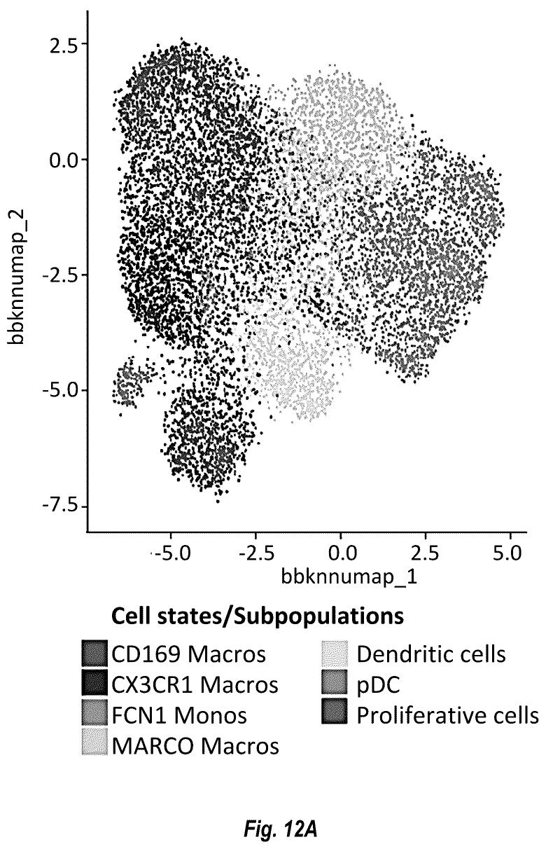

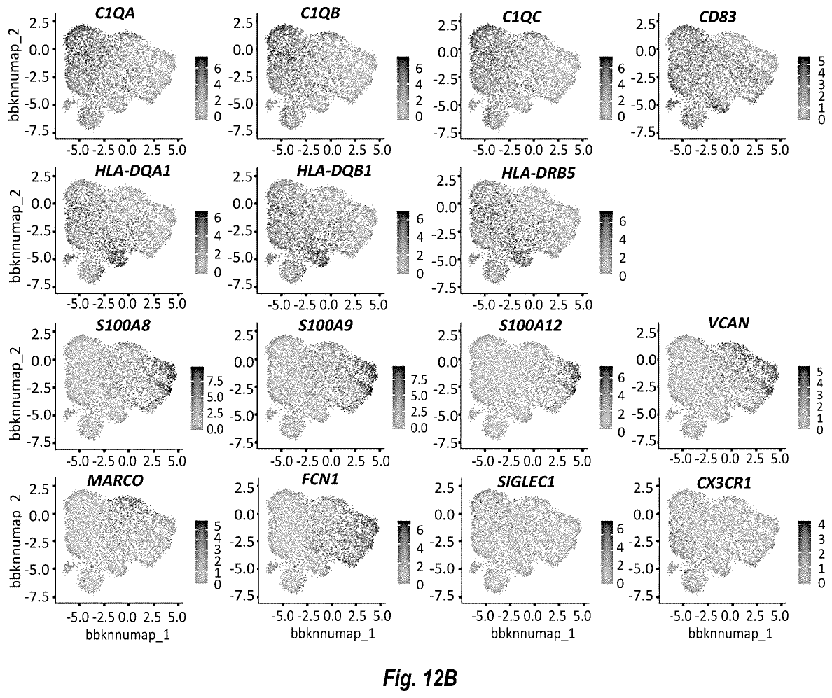

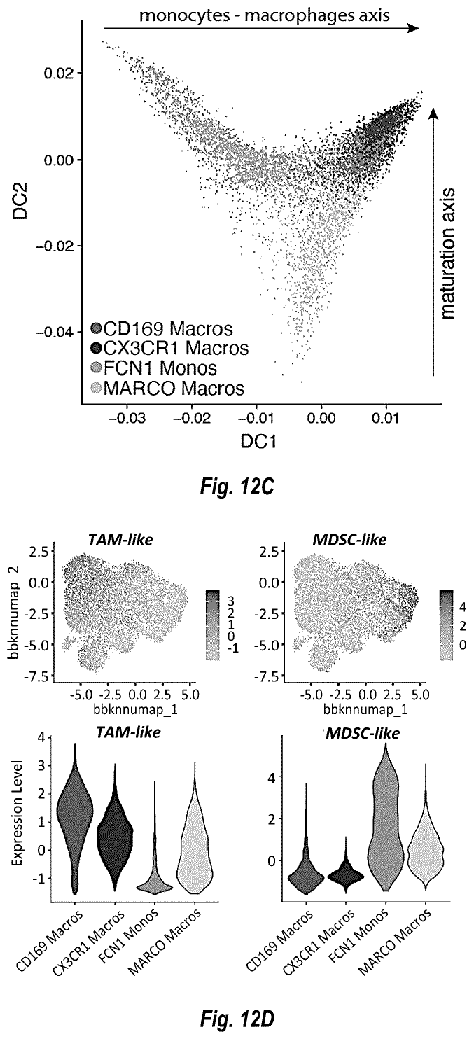

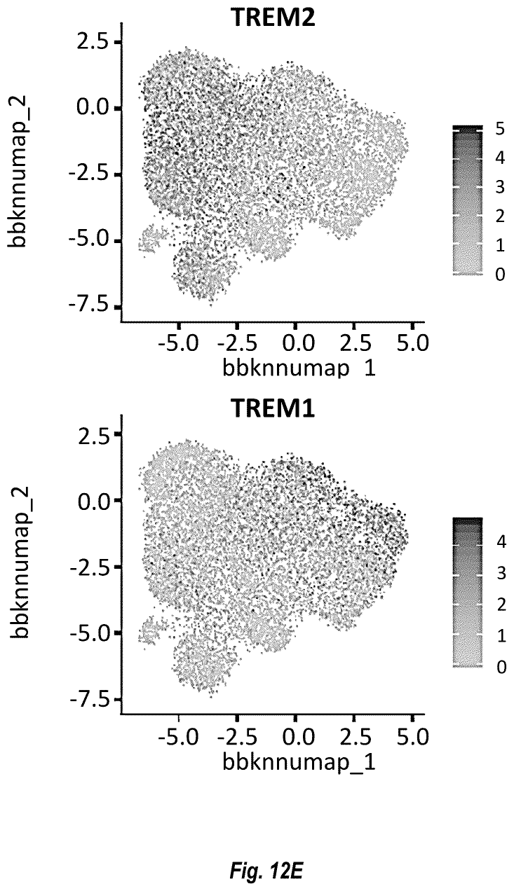

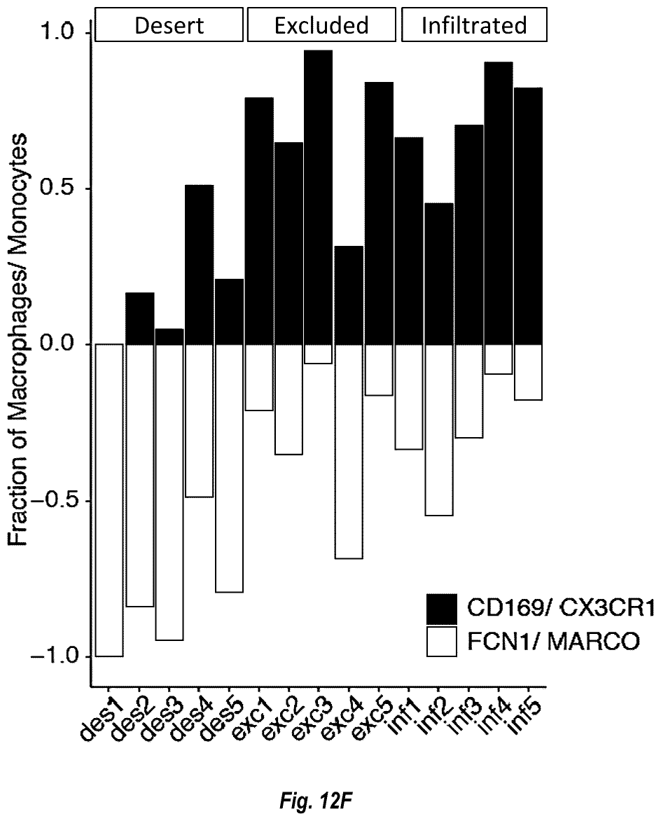

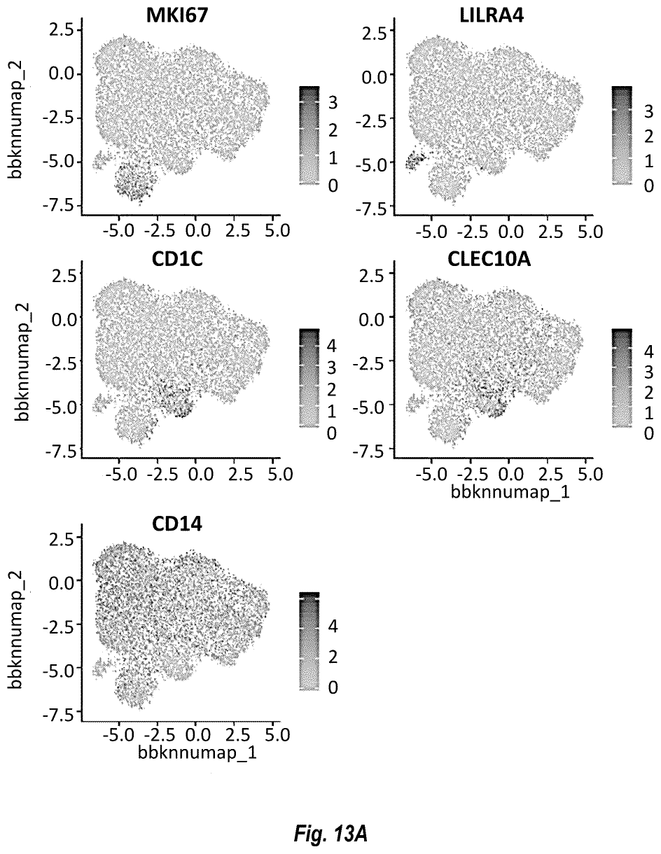

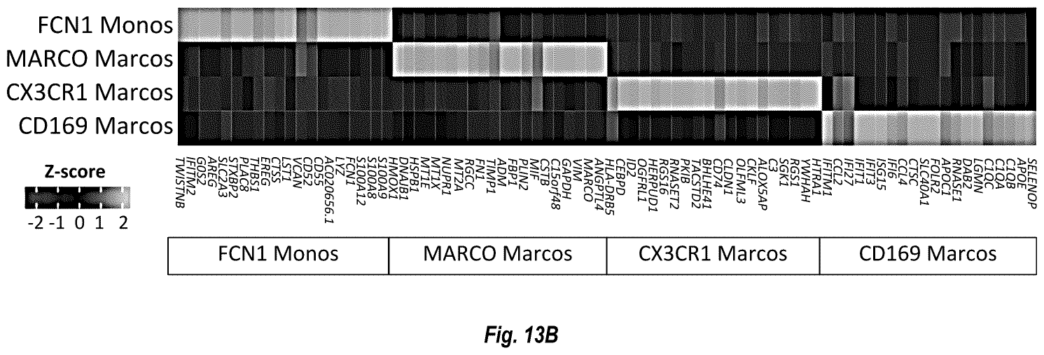

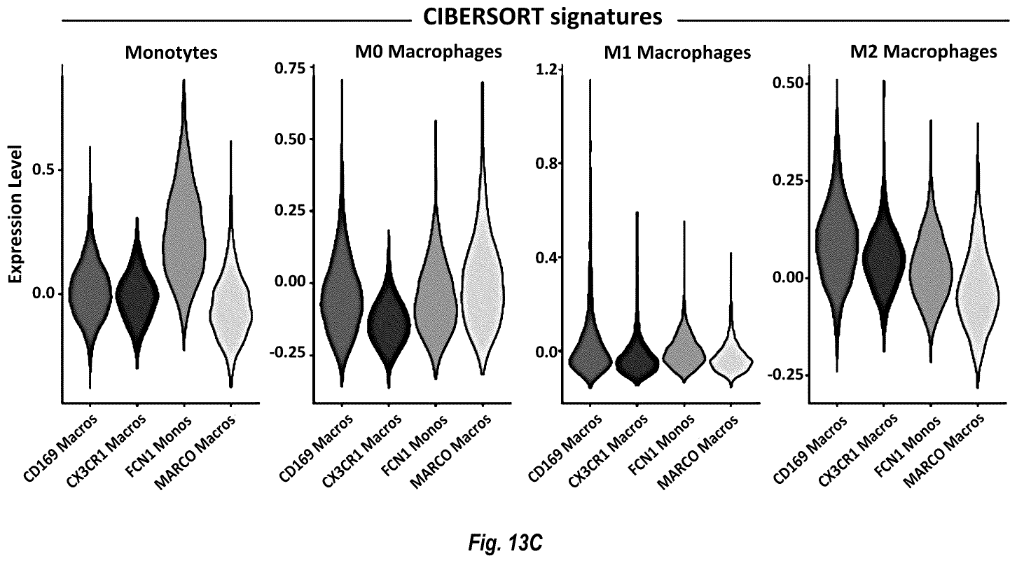

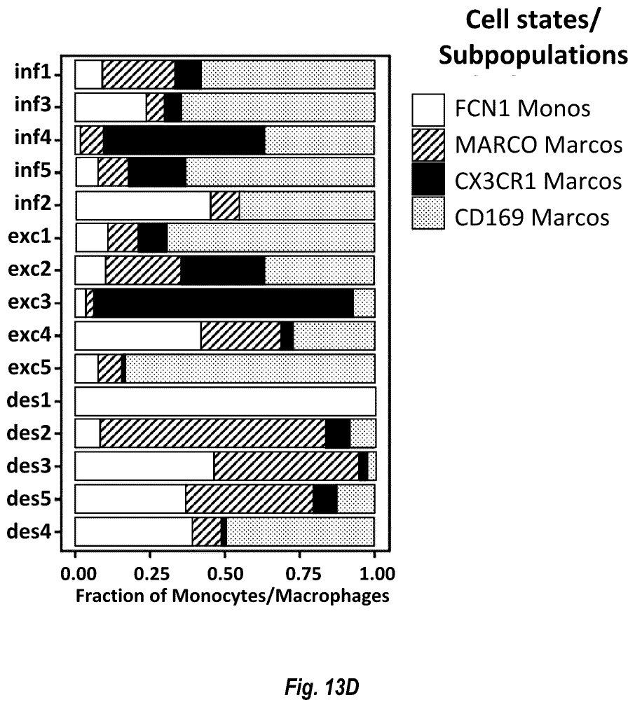

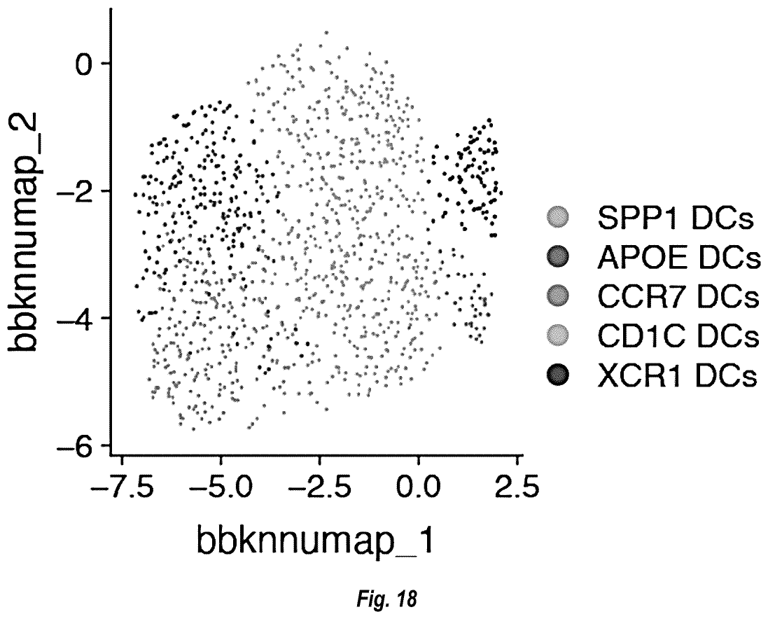

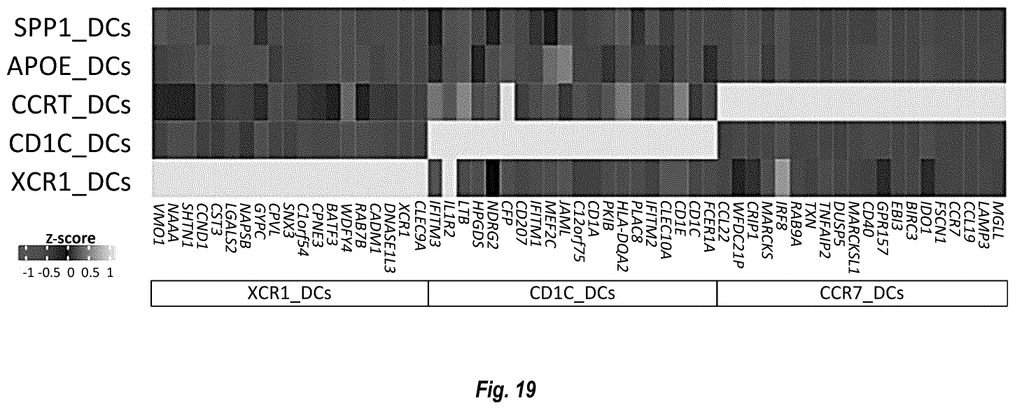

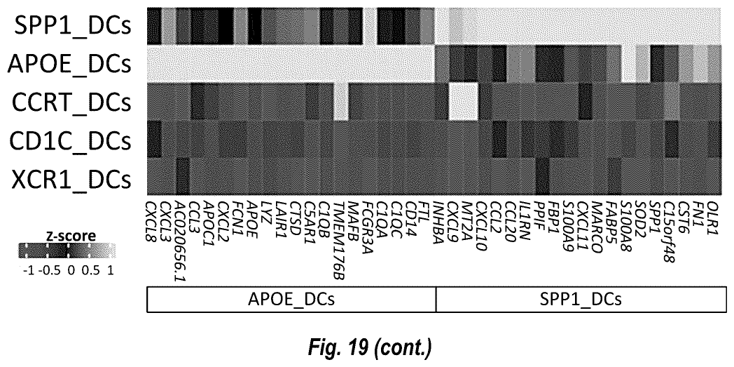

Analysis of the myeloid compartment (FIG. 7A) identified clusters representative of dendritic cells (DC) marked by CD1C and CLEC10A expression, plasmacytoid dendritic cells (pDC) expressing LILRA4, and four macrophage/monocyte clusters marked by CD14 expression (FIGS. 12A and 13A). A subcluster analysis of the DC population identified five clusters labeled according to the featured genes including: i) SPP1 DCs, ii) APOE DCs, iii) CCR7 DCs, iv) CD1C DCs and v) XCR1 DCs (FIG. 18). Three of these clusters can be confidently assigned to previously described DC subsets with XCR1 DCs representing the cDC1 population (XCR1, CLEC9A, CADM1), the CDC1 DCs as the cDC2 population (FCER1A, CD1C, CLEC10A) and the CCR7 DCs representing mature DCs (CCR7, LAMP3) (Zhang et al., 2019). Based on the expression of MAFB, FCGR3A, CD14, FCGR1A and CD163, we hypothesized that the APOE DCs cluster represents an inflammatory monocyte-derived DC population (Collin and Bigley, 2018), while the SPP1 DCs shared similarities with the CD1c− CD141− DC subset described by Villani et al. (SERPINA1 and FCGR3A) (Villani et al., 2017) (FIG. 19). Unfortunately, the number of DCs was too low to investigate the association of these DC subsets with the tumor immune phenotypes. Further work is warranted to validate the function of these cells and their distribution in the TME.

We further characterized the four macrophage/monocyte clusters according to gene markers that have been used previously to describe similar cell populations: FCN1, MARCO, SIGLEC1 (CD169) and CX3CR1 (FIGS. 12A and 12B). Aran, D. et al., Nat. Immunol., 20:163-172 (2019); Azizi, E. et al., Cell, 174:1293-1308-e36 (2018); Cassetta, L. et al., Cancer Cell, 35:588-602.e10 (2019); Lavin, Y. et al., Cell, 169:750-765.e17 (2017); Zilionis, R. et al., Immunity, 1-29, doi:10.1016/j.immuni.2019.03.009 (2019). The FCN1 cluster was characterized by high expression of VCAN and S100A transcripts that were previously associated with monocytes (FIGS. 12B and 13B). Villani, A.-C., et al., Science, 356(6335):eaah4573, doi:10.1126/science.aah4573 (2017); Zilionis, R. et al., Immunity, 1-29, doi:10.1016/j.immuni.2019.03.009 (2019). Moreover, this cluster exhibited the highest expression of the CIBERSORT monocyte signature (FIG. 13C). Since the CD169 and CX3CR1 populations highly expressed complement factor genes, maturation markers (CD83, HLA-DQA1, HLA-DQB1, HLA-DRB5) and the M2 CIBERSORT signature, we characterized these populations as tumor-associated macrophage (TAM)-like macrophages (FIGS. 12B, 13B, and 13C). Finally, the MARCO cluster was characterized by lower VCAN expression compared to the FCN1 cluster and lower M2 signature and maturation marker expression compared to the TAM-like macrophages. We therefore annotated these cells as MARCO macrophages similar to the previously described population. Zilionis, R. et al., Immunity, 1-29, doi:10.1016/j.immuni.2019.03.009 (2019). To interrogate the differentiation trajectory of the identified cell states, we performed a pseudotime diffusion map analysis. The trajectory along the diffusion map coordinate 2 follows a continuum from MARCO immature macrophages to CD169 and CX3CR1 mature macrophages, suggesting a maturation trajectory, while the diffusion map coordinate 1 reflects a differentiation trajectory from FCN1 monocytes to mature CD169/CX3CR1 macrophages (FIG. 12C). To further investigate the myeloid cell states, we derived and applied signatures of recently described TAM-like and MDSC-like cells in hepatocellular carcinoma. Zhang, Q. et al., Cell 179:829-845 (2019). Indeed, the TAM-like macrophage signature was expressed in the CD169 and CX3CR1 macrophages and the MDSC-like signature was highly expressed in the FCN1 monocytes and MARCO macrophages (FIG. 12D). Differential expression of two members of the Triggering Receptor Expressed on Myeloid cells (TREM) family, TREM1 and TREM2, has previously been associated with the TAM-like and MDSC-like states. Zhang, Q. et al., Cell 179:829-845 (2019). Consistent with this, we found TREM2 to be almost exclusively expressed in the TAM-like CD169/CX3CR1 macrophages, and TREM1, in the MDSC-like FCN1 monocytes/MARCO macrophages (FIG. 12E).

Finally, we evaluated whether these different myeloid cell populations were associated with particular tumor immune phenotypes. The MDSC-like myeloid subset (FCN1 monocytes and MARCO macrophages) were significantly enriched in desert tumors, whereas the TAM-like myeloid subset (CD169 and CX3CR1 macrophages) was enriched in excluded and infiltrated tumors (p=0.02, FIGS. 12F and 13C). Of note, no difference were observed between excluded and infiltrated tumors for either population.

### Example 6. Tumor Immune Phenotypes are Shaped by Cross-Compartment Interactions

### 6.1. Pseudo-Bulk Differential Expression

We used a pseudo-bulk approach to perform differential gene expression (DGE) analysis. For each sample, raw UMI counts for each gene were summed across cells of a cell population of interest derived from that sample, resulting in sample-level UMI counts. Samples with fewer than five cells and genes with less than 50 reads across samples were excluded from the analysis. We then calculated the size factors for each pseudo-bulk sample using calcNormFactors (edgeR) and used voom-limma to perform differential gene expression analysis on these sample-level pseudo-bulk expression profiles. For the G2/M corrected pseudo-bulk expression analysis in the tumor compartment, we calculated a G2/M score per cell using the CellCycleScoring function with G2/M cell cycle genes as previously described (Seurat, science.sciencemag.org/content/352/6282/189). The per cell scores were averaged by patient and added to the voom-limma design matrix as a covariate.

### 6.2. Gene Set Enrichment Analysis

Gene set enrichment analysis was performed on the results of the differential gene expression analysis using the fgsea package and the hallmark gene set from the molecular signatures database collection using the msigdbr package. Korotkevich, G. et al., bioRxiv 060012; doi:10.1101/060012 (2019); Subramaniam, A. et al., Proc. Natl. Acad. Sci. U.S.A., 102:15545-15550 (2005). In detail, the differentially expressed gene list was ranked according to the combined log fold change and adjusted p-value and used as an input for the gene set enrichment analysis.

### 6.3 Chemokine Receptor-Ligand Interaction Analysis

A database of known chemokine receptor and ligand pairs was curated using the combined information from cellphoneDB (Efremova et al., 2020) (cellphonedb.org/) and resources from the R&D systems website (rndsystems.com/resources/technical-information/chemokine-nomenclature). This database was filtered for possible chemokine-receptor interactions using the following criteria: 1) each receptor-ligand pair should be expressed in at least 10% of cells of a cell population, 2) each pair should be expressed within the same immune phenotype. This resulted in 6 possible chemokine receptor-ligand interactions, for which the expression levels and the number of cells expressing a receptor/ligand was assessed in each individual patient.

### 6.4 Results

Our single cell characterization of the tumor microenvironment also allowed us to interrogate potential interactions between cell compartments that may help to shape the tumor immune continuum. Although single-cell data cannot provide definitive evidence of cell-to-cell signaling, various groups have shown how consideration of cell-specific receptor and ligand expression patterns in single-cell data can be used to fruitfully generate hypotheses. Camp, J. G. et al., Nature, 546:533-538 (2017); Costa, A. et al., Cancer Cell, 33:463-479.e10 (2018); Skelly, D. A. et al., Cell Reports, 22:600-610 (2018); Vento-Tormo, R. et al., Nature, 563:347-353 (2018). Following such approaches, we focused on 23 known chemokine ligand-receptor pairs and determined which cell types express a chemokine ligand or receptor; for implicated cell types, we further noted the expression level and fraction of cells expressing the receptor or ligand. We excluded any putative interaction pair for which 1) the receptor was expressed by less than 1% of cells of a cell type or subpopulation and 2) the receptor and ligand expression did not co-occur in the same patient for the majority of the patients. This filtering resulted in six putative chemokine ligand-receptor interactions that we further investigated in our dataset. Intriguingly, we found evidence potentially implicating each of the compartments (tumor, immune and stromal) in the recruitment and migration of T cells.

TABLE 1
Hallmark Gene Set Enrichment Results Based on the Differentially Expressed
Genes in Desert vs Infiltrated/Excluded Tumor Cells

| | Adjusted | | |
|---|---|---|---|
| Pathway | p-value | NES | Gene set-leading Edge genes |
| HALLMARK_ | 0.0129 | 1.611 | CDKN2A, CCL4, B2M, TAP1, HLA-DQA1, CTSS, EREG, IL2RG, STAB1, |
| ALLOGRAFT_ | | | CD247, HLA-DOB, MMP9, TAP2, LTB, GBP2, ICAM1, CCR1, HLA-A, STAT1, |
| REJECTION | | | PRKCG, ELF4, HLA-G, CSF1, CD47, RPL3L, NCK1, IL27RA, IL15, CD40, |
| | | | TPD52, TAPBP, JAK2, C2, CSK, CAPG, LYN, TRAF2 |
| HALLMARK_ | 0.0337 | −1.685 | SPP1, SERPINA5, STC1, VCAN, FGFR1, NRP1, FSTL1 |
| ANGIOGENESIS | | | |
| HALLMARK_ | 0.0129 | 1.512 | CDKN2A, CDKN2C, CDC20, SUV39H1, JPT1, ASF1B, CDCA8, DDX39A, |
| E2F_TARGETS | | | TACC3, KIF2C, TK1, CTPS1, TRIP13, CCNE1, BIRC5, SPC24, MYC, CCNB2, |
| | | | CKS1B, POLD1, KPNA2, HUS1, PLK1, TCF19, AURKA, MELK, ORC6, |
| | | | UBE2S, RAD1, DEPDC1, CHEK2, AK2, CDKN3 |
| HALLMARK_ | 0.0129 | −1.749 | COL1A2, SPP1, DAB2, IGFBP4, WNT5A, MMP2, ACTA2, BGN, ADAM12, |
| EPITHELIAL_ | | | VIM, VCAN, BASP1, COL6A2, BMP1, FBN2, PMP22, SPARC, SERPINE2, |
| MESENCHYMAL_ | | | FBLN1, FBN1, LAMA1, CRLF1, LOXL2, COL4A1, SLIT2, FSTL1, SFRP1, |
| TRANSITION | | | FGF2, FBLN2, RGS4, CDH2, TGFB1, HTRA1, FOXC2, FMOD, GJA1, PRRX1, |
| | | | COL4A2, VEGFC, CDH6, SGCD, FN1, COL5A2, PDGFRB, SLIT3, EMP3, |
| | | | TIMP3, IGFBP2, COL6A3, GAS1, EFEMP2, MATN3, PTX3, DST, SERPINE1, |
| | | | PDLIM4, CAP2, SFRP4 |
| HALLMARK_ | 0.0035 | 2.056 | EPSTI1, ISG15, CMPK2, CXCL10, IFI44, CXCL11, BATF2, SAMD9, RTP4, |
| INTERFERON_ | | | PARP9, MX1, IFIH1, OASL, SAMD9L, B2M, IFIT3, TAP1, ADAR, PSMB9, |
| ALPHA_RESPONSE | | | PLSCR1, C1S, RSAD2, HLA-C, TDRD7, USP18, GBP2, IFIT2, UBE2L6, |
| | | | TMEM140, PARP12, HELZ2, CASP1, SP110, GBP4, PARP14, LAP3, IFI44L, |
| | | | CSF1, PROCR, NMI, IFI27, CD47, LGALS3BP, PSMB8, PSME2 |
| HALLMARK_ | 0.0035 | 2.130 | IDO1, EPSTI1, ISG15, CMPK2, IFIT1, NLRC5, CXCL10, ZBP1, IFI44, CXCL11, |
| INTERFERON_ | | | BATF2, VCAM1, RTP4, MX1, IFIH1, SAMHD1, CD274, OASL, IL10RA, |
| GAMMA_ | | | OAS3, SAMD9L, AC124319.1, MX2, B2M, IFIT3, TAP1, SECTM1, ADAR, |
| RESPONSE | | | HLA-DQA1, PDE4B, PSMB9, HLA-B, OAS2, PLSCR1, C1S, RSAD2, TDRD7, |
| | | | MYD88, TNFSF10, CIITA, USP18, DDX58, CFB, ICAM1, IFIT2, UBE2L6, |
| | | | PARP12, C1R, HELZ2, CASP1, SP110, HLA-A, GBP4, PARP14, STAT1, |
| | | | IL18BP, CD38, PSMB2, LAP3, IFI44L, HLA-G, NMI, IFI27, TNFAIP6, MT2A, |
| | | | BPGM, LGALS3BP, SOD2, PSMB8, PSME2, XAF1 |
| HALLMARK_ | 0.0361 | 1.446 | ATP6V0B, PDP1, COX7B, PDHA1, UQCRFS1, OPA1, MRPS12, MRPS15, |
| OXIDATIVE_ | | | NDUFS6, CYC1, SDHB, NDUFA1, NDUFB7, NDUFS2, SDHA, TIMM17A, |
| PHOSPHORYLATION | | | MFN2, MDH2, HSD17B10, MRPS11, HCCS, MRPL35, SLC25A20, PHYH, |
| | | | NQO2, NDUFA3, ATP6AP1, BCKDHA, OGDH, IDH2, NNT, RETSAT, COX6C, |
| | | | FDX1, COX5A, ATP6V1C1, IDH1, NDUFB2, ETFA, TCIRG1, POR, NDUFA8, |
| | | | IDH3A, ECH1, AIFM1, ISCA1, COX8A, NDUFC2, NDUFS8, UQCR11, |
| | | | MRPL15, ATP1B1, COX5B, DLD, NDUFB4, FH, ETFB, ATP6V1F, NDUFA6, |
| | | | COX17, ATP6V0E1, ECI1, COX6A1, TIMM50, ATP6V1E1, NDUFS7, |
| | | | NDUFB3, MGST3, SDHC, NDUFS3, CYCS, SURF1, ATP6V1G1, TIMM8B |

### 6.4.1 Tumor Cell-Immune Cell Crosstalk

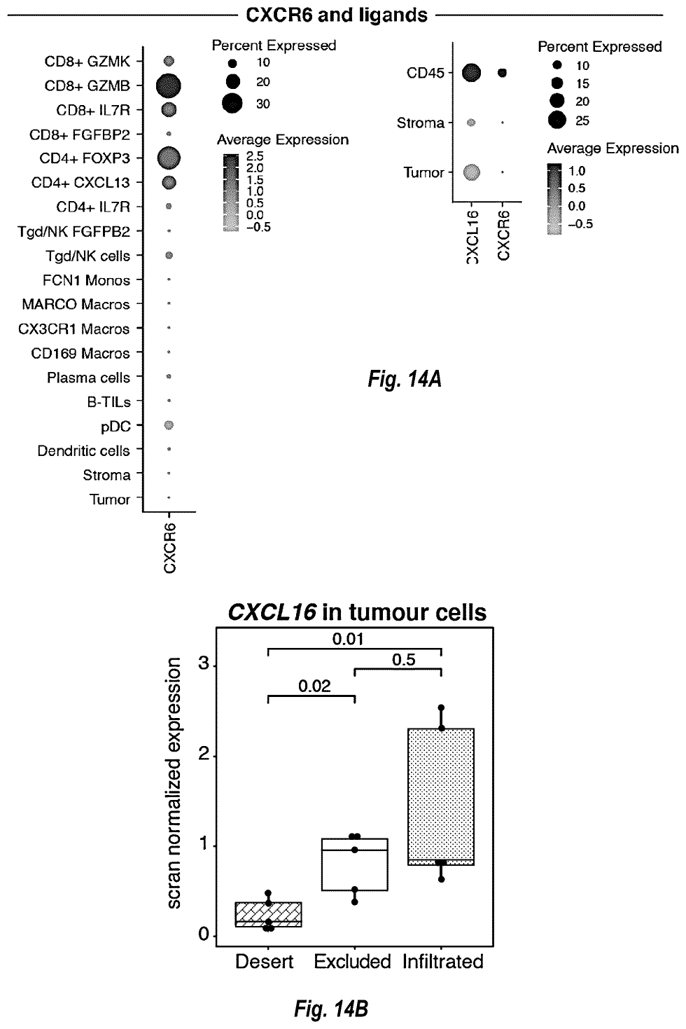

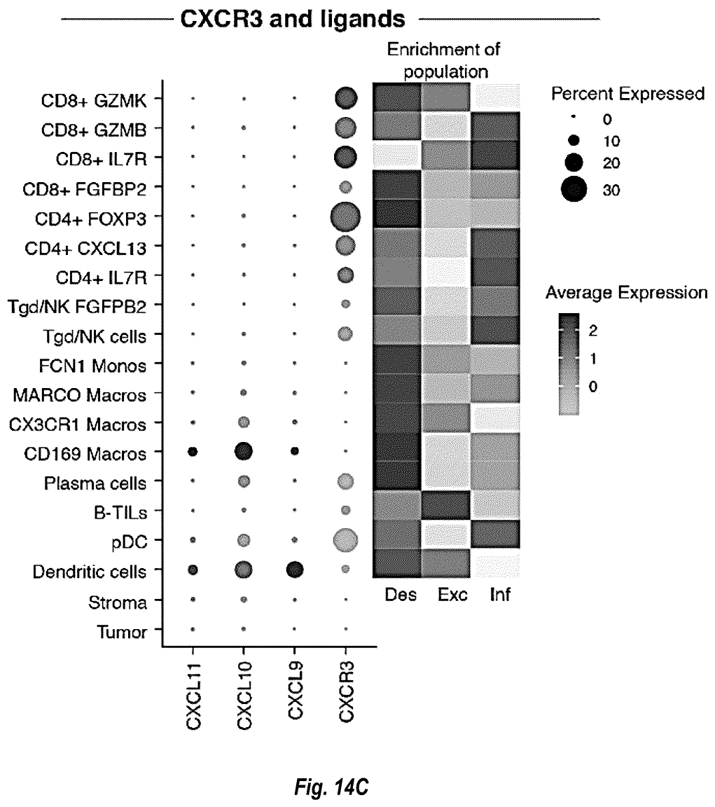

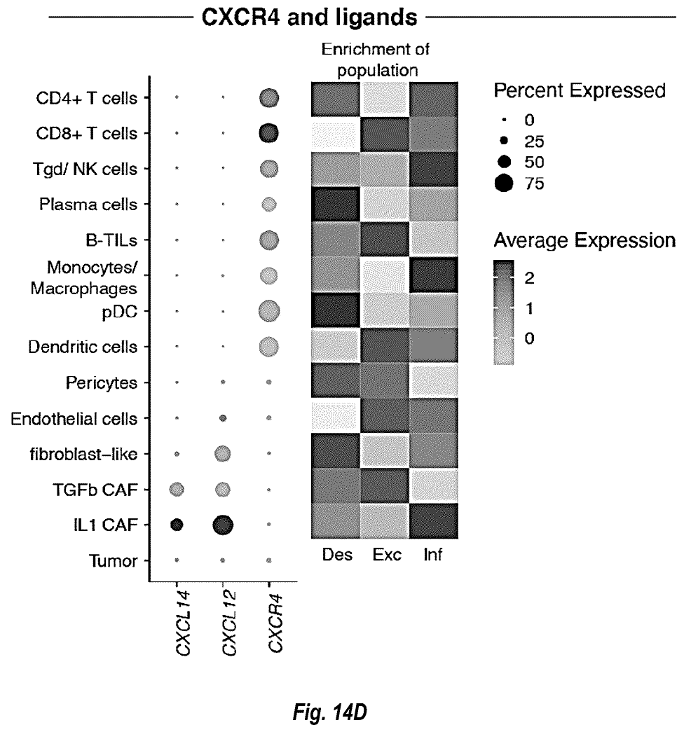

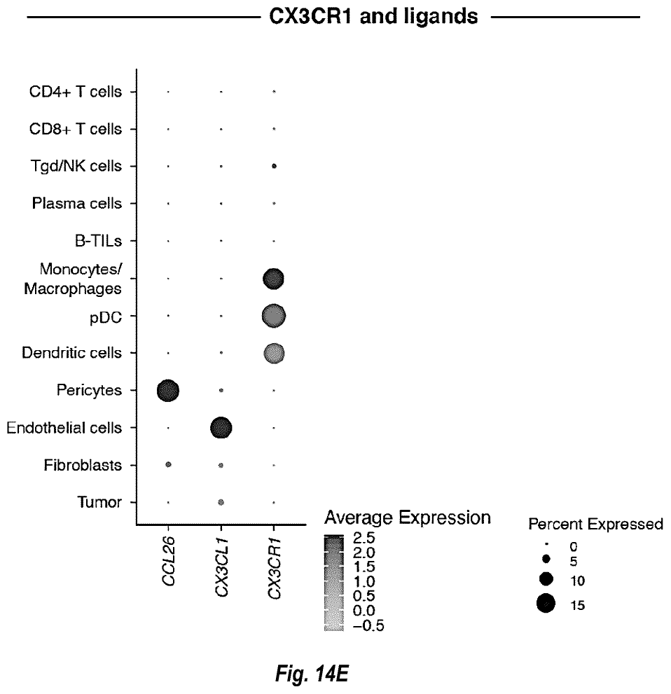

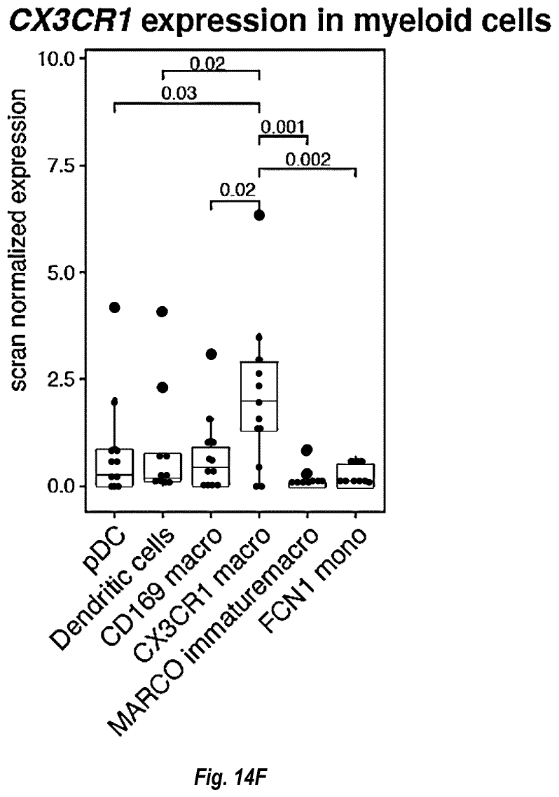

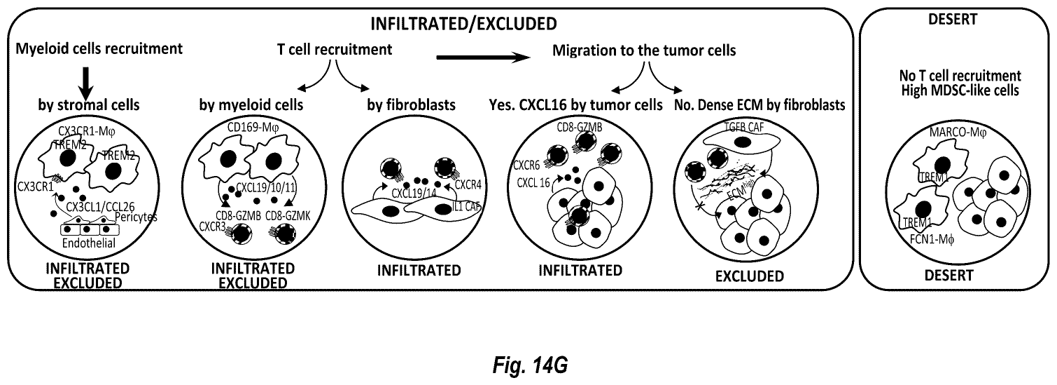

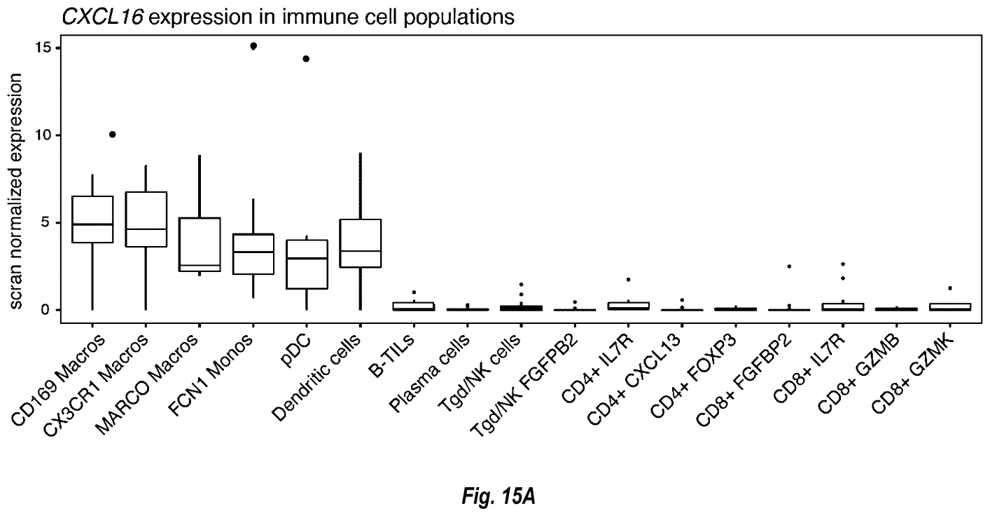

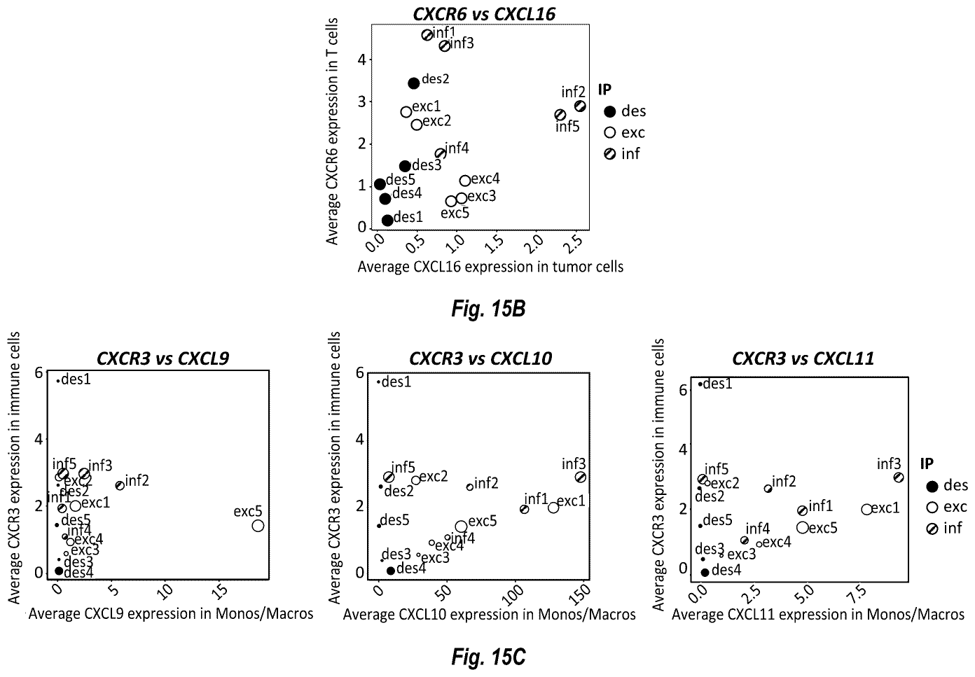

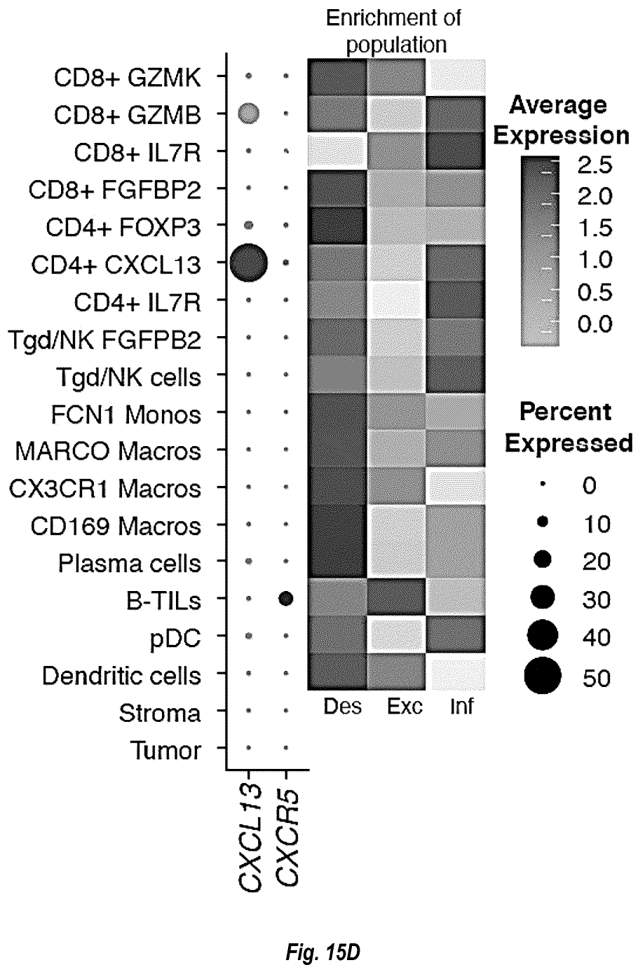

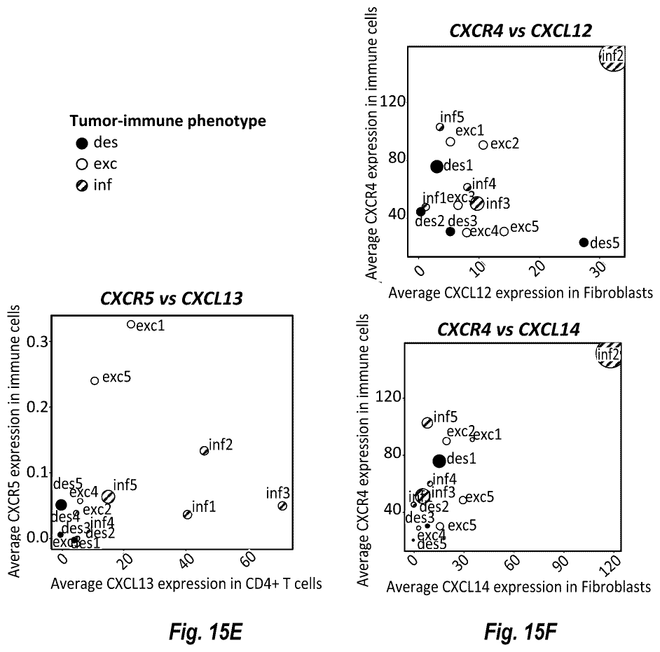

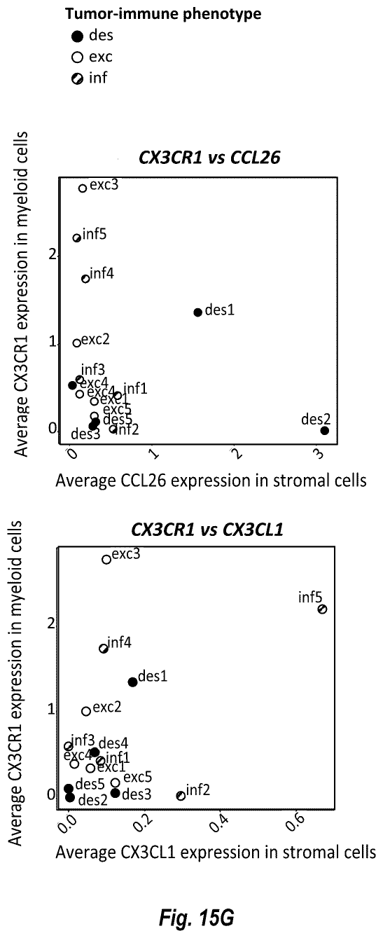

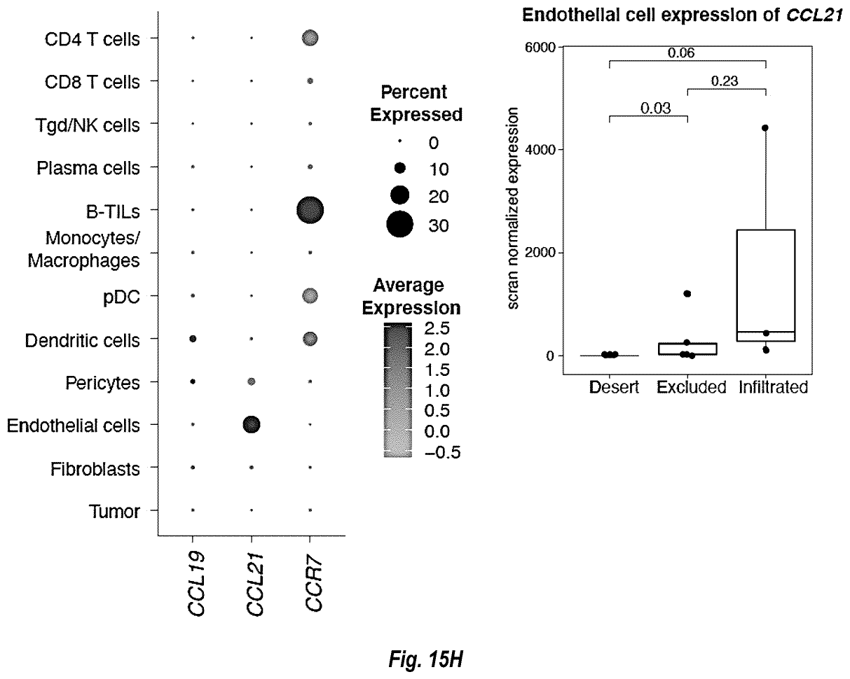

The chemokine CXCL16, a chemokine known for the recruitment of T cells, was expressed by tumor cells as well as immune cells mostly myeloid cells (FIGS. 14A and 15A). Matsumara, S. and Demaria, S. Radiat. Res., 173:418-425 (2010). Further, we observed that CXCL16 expression was significantly higher in the tumor cell compartment of infiltrated and excluded tumors compared to desert tumors (p-value=0.012 and 0.022, respectively, FIG. 14B). On the other hand, the CXCL16 receptor, CXCR6, was found to be specifically expressed in CD4+ and CD8+ T cells and particularly high in CD8+ GZMB T cells and CD4+ FOXP3 Tregs (FIG. 14A). Wilbanks, A. et al., J. Immunol., 166:5145-5154 (2001). This co-expression pattern could be confirmed in all individual patients for which we had enough cells, and it was particularly apparent in tumor cells of infiltrated tumors (FIG. 15B). Together with our observation that CD8+ GZMB T cells and CD4+ FOXP3 Tregs were enriched in infiltrated tumors, this finding suggested a potential mechanism by which CXCR6 T cells could be recruited to the tumor epithelium in infiltrated tumors.

### 6.4.2. Immune Cell-Immune Cell Crosstalk

Similar to the potential CXCL16/CXCR6 crosstalk between tumor cells and T cells, we identified possible crosstalk between T cells and myeloid cells. The CXCR3 receptor was expressed by CD8+ and CD4+ T cells, and its major ligands, CXCL9, CXCL10 and CXCL11 were mainly expressed by dendritic cells and CD169 macrophages (FIGS. 14C and 15C). In addition, the expression of CXCR5 by B-TILs and of the corresponding chemokine ligand CXCL13 by CD4+ CXCL13 and CD8+ GZMB T cells in infiltrated tumors suggests a potential mechanism of B-TILs recruitment to infiltrated tumors (Kazanietz, M. G. et al., Front Endocrinol (Lausanne) 10, 471 (2019)) (FIGS. 15D-E).

### 6.4.3. Stromal Cell-Immune Cell Crosstalk

Stromal cells may also participate in the recruitment of immune cells. The chemokines CXCL14 and CXCL12 were expressed by the IL1 CAF population (FIG. 14D), and they are known to bind to the receptor CXCR4. Collins, P. J. et al., FASEB J., 31:3084-3097 (2017); Tanegashima, K. et al., FEBS Letters, 587:1731-1735 (2013); Vega, B et al., Journal of Leukocyte Biology, 90:399-408 (2011). Notably, this receptor was found to be almost exclusively expressed by immune cells, and most strongly by CD8+ T cells (FIGS. 14D and 15F), suggesting a role for IL1 CAFs in the CD8+ T cell recruitment in infiltrated tumors since these show a weak enrichment of IL1 CAFs while T cells are rare in desert tumors.

Endothelial cells and pericytes were the only cell types to appreciably express the main ligands of CX3CR1: CX3CL1 and the recently described ligand CCL26 (FIG. 14E). El-Shazly, A. E. et al., Clin. Exp. Allergy, 43:322-331 (2013). Since CX3CR1 marked one of the major mature macrophage populations in infiltrated and excluded tumors (FIG. 14F), we hypothesize that a recruitment of CX3CR1 macrophages through endothelial cells and pericytes is specific to these two immune phenotypes (FIG. 15G).

In addition to myeloid cells, B-TILs showed evidence of possible chemokine receptor-ligand crosstalk with the stromal cell compartment. Endothelial cells expressed CCL21, a ligand enabling the recruitment of CCR7+ cells, with a significantly higher expression in endothelial cells of excluded tumors (p=0.03) and consistent but non-significant higher expression in infiltrated tumors (p=0.06, FIGS. 15H and 15I). Comerford, I. et al., Cytokine Growth Factor Rev. 24:269-283 (2013). In our dataset, most CCR7-expressing cells are B-TILs, suggesting possible recruitment via endothelial cells, specifically in excluded and infiltrated tumors.

### 7. Conclusions

To summarize these observations, we postulate a model in which the different composition of and the potential crosstalk within and between the tumor, immune and stromal compartments might shape the distinct tumor immune phenotypes (FIG. 14G). In our model, tumors with immune presence in the TME, including both infiltrated and excluded tumors, share many common features that are distinct from desert tumors. For example, both infiltrated and excluded tumors showed an enrichment of T cells and TAM-like macrophages, while desert tumors show an almost complete absence of T cells, but an enrichment of MDSC-like cells. The T cell presence in infiltrated and excluded tumors could be in part facilitated by a T cell-TAM-like macrophage crosstalk through CXCR3-CXCL9/10/11 signaling. Additionally, endothelial cells and pericytes expressing CX3CR1 ligands might also participate in the recruitment of CX3CR1 TAM-like macrophages in infiltrated and excluded tumors. On the other hand, there were also important differences between infiltrated and excluded tumors. The greater extent of T cell infiltration in the infiltrated tumors might be influenced by two factors: 1) infiltrated tumor cells showed the highest CXCL16 expression, which may promote the recruitment of CXCR6+ T cells to the tumor epithelium; and 2) infiltrated tumors are enriched in IL1 CAFs, whose expression of CXCL12/14 may further facilitate T cell recruitment via CXCR4.

### 8. Discussion

While cancer immunotherapy is effective in certain indications, even there it is not effective for everyone and variability in response is not completely understood. Sharma, P. et al., Cell, 168:707-723 (2017). Developing a better understanding of the composition of the tumor and its microenvironment should in turn improve and extend the benefit of cancer immunotherapies to more patients and enable personalized approaches to treatment. Hegde, P. S. and Chen, D. S., Immunity, 52:17-35 (2020). In this study, we report a comprehensive scRNAseq dissection of the entire tumor ecosystem in the context of the three tumor immune phenotypes that comprise the tumor immunity continuum in ovarian cancer. Our study not only provides a high-resolution depiction of the cellular diversity in each of the tumor, immune and stromal compartments, but it also highlights how the crosstalk within and between the compartments may contribute to shaping the biology of tumor immune phenotypes and ultimately could influence the response to immunotherapies.

We first investigated whether intrinsic properties of tumor cells themselves contribute to a milieu that favors or hinders immune infiltration. Galon, J. and Bruni, D., Nat Rev Drug Discov, 18:197-218 (2019). Indeed, scRNAseq of the tumor cell compartment revealed significant differences in the transcriptional profiles between desert and infiltrated/excluded tumors such as an enrichment of the interferon response/antigen presentation pathway, representative of high CD8+ T cell infiltration, in the infiltrated/excluded tumors. Downregulation of antigen presentation has been observed in our previous bulk RNA sequencing and in situ MHC-I IHC studies in desert and excluded tumors. Desbois, M et al., Nature Communications, 11:5583, doi:10.1038/s41467-020-19408-2 (2020). Although we did not observe a strong downregulation of antigen presentation at the gene level in the excluded tumors in this study, we cannot rule out that MHC may be downregulated at the protein level. Another feature characterizing infiltrated/excluded tumors was upregulation of the oxidative phosphorylation pathway. It has been previously reported that metabolism of tumor cells can directly impair T cell infiltration and function through, for example, competition for metabolites that are essential for T cell function, or accumulation of waste products like lactate that create an unfavorable microenvironment and impair T cells migration. Haas, R. et al., PLoS Biol, 13:e1002202, (2015); Sugiura, A. and Rathmell, J. C., J Immunol, 200:400-407 (2018). Using oxidative phosphorylation instead of aerobic glycolysis, tumor cells consume pyruvate, and less lactate is accumulated (Sugiura, A. and Rathmell, J. C., J Immunol, 200:400-407 (2018)), and this may generate a more favorable milieu for effective immune cell infiltration.

The tumor immunity continuum has largely been characterized in terms of the quantity and location of T cells in the tumor bed. Hegde, P. S. et al., Clinical Cancer Research, 22:1865-1874 (2016). Our single-cell interrogation of the immune compartment provided far more detail and revealed diverse phenotypic and functional T cell and myeloid cell states, as well as their differential enrichment in the tumor immune phenotypes. While the CD8+ GZMB and CD8+ GZMK T cell subpopulations we identified have been previously described in single-cell studies (Guo, X. et al., Nature Medicine, 24:978-985 (2018); Li, H. et al., Cell, 176:77-789, doi:10.1016/j.cell.2018.11.043 (2019); Wu, T. D. et al., Nature, 579:274-278 (2020); Yost, K. E. et al., Nature Medicine, 25:1251-1259 (2019); Zhang, L. et al., Nature, 54:321-33 (2018); Zhang, Q. et al., Cell 179:829-845 (2019), one of our key findings is that they not only represent different functional states, but are also differentially enriched in the different tumor immune phenotypes. Our analysis of the association of these CD8+ T cell subpopulations with the tumor immune phenotypes provided two insights: 1) the dysfunctional/exhausted CD8+ GZMB T cells were less activated/exhausted in excluded tumors; and 2) the pre-dysfunctional/effector memory CD8+ GZMK T cells were enriched in excluded tumors compared to infiltrated tumors. Both observations might be explained by a potential link between the dysfunction/activation states and T cell spatial distribution, i.e., infiltration or exclusion of the T cells from the tumor epithelium. The immune cell exclusion in excluded tumors might result in a lack of sustained antigenic stimulation by tumor cells and therefore contribute to the less activated/exhausted CD8+ GZMB T cells and the enrichment of pre-dysfunctional CD8+ GZMK T cells. In line with this model, we found an enrichment of resting CD4+ T cell populations in the excluded tumors, and activated CD4+ T cells and regulatory T cells in the infiltrated tumor. Taken together, these observations point towards a more antigen-stimulated immune landscape in infiltrated compared to excluded tumors, as one would expect. However, our spatial analysis also suggests that the pre-dysfunctional CD8+ GZMK T cell population has the capability of infiltrating the tumor epithelium. Therefore, the spatial localization, i.e. the exclusion of the CD8+ GZMK T cells from the tumor epithelium, cannot fully explain their functional state.

One important question being investigated by many groups is whether immune checkpoint blockades (ICB), in particular anti-PD-(L)1 antibodies, can reinvigorate the dysfunctional tumor-infiltrating CD8+ T cells. While additional investigation is needed, recent studies support a model where pre-dysfunctional rather than late dysfunctional T cells are targeted by ICB and promote anti-tumor response. In particular, the Tscm cells belonging to the pool of pre-dysfunctional T cells have been found to expand upon ICB treatment (Sade-Feldmanm, M. et al., Cell, 175:998-1013 e1020 (2018); Utzschneider, D. T. et al., Immunity, 45:415-427 (2016)) and to associate with a longer duration of response to ICB treatment (Miller, B. C. et al., Nat Immunol 20:326-336 (2019)). While our understanding of the role of these cells is increasing in the context of immunotherapies, many questions remain on their role in other cancer therapies, such as chemotherapy used in ICON7. Hence, the mere presence of Tscm cells in our cohort of ovarian cancer patients might not impact their survival. Moreover, in our previous work (Desbois, M et al., Nature Communications, 11:5583, doi:10.1038/s41467-020-19408-2 (2020)) and in this present study, we observed that the quantity of CD8+ TILs infiltrating the tumor is only weakly associated with PFS. Patients with excluded tumors showed shorter PFS, and the amount of CD8+ GZMK cells among all CD8+ cells predicted shorter PFS under chemotherapy. Because we found these pre-dysfunctional cells enriched in excluded tumors it is possible that the observed association with PFS is intimately linked. Although the presence of pre-dysfunctional cells is inversely correlated with PFS in a chemo cohort, these observations do not rule out an optimal clinical efficacy if both activated stroma and pre-dysfunctional/Tscm cells are simultaneously targeted under ICB.

Another key finding of this study is the in-depth dissection of the heterogeneous myeloid cell population in the context of different tumor immune phenotypes. We identified four myeloid cell states: FCN1 monocytes, and MARCO, CD169 and CX3CR1 macrophages, with a spectrum of maturation, from MDSC-like FCN1 monocytes and MARCO immature macrophages to the more mature, TAM-like CD169 and CX3CR1 macrophages. Whereas desert tumors were enriched in MDSC-like myeloid cells (FCN1 and MARCO) infiltrated/excluded tumors were enriched in TAM-like myeloid cells. One possible explanation of the observed link between the maturation levels of myeloid cells and the different tumor immune phenotypes might be the interferon levels in the TME. Interferon signaling has been shown to play a major role in the activation of macrophages, their antigen presentation activity and their pro-inflammatory functions. Hu, X. and Ivashkiv, L. B., Immunity, 31:539-550 (2009). The main producers of IFNγ are immune cells, including Th1 CD4+ T cells, cytotoxic CD8+ T cells and NK cells. Schoenborn, J. R. and Wilson, C. B., Adv Immunol, 96:41-101 (2007). Hence, in excluded and infiltrated tumors, infiltrating immune cells can promote the maturation of myeloid cells through their interferon production. Supporting this hypothesis, we found that the interferon response pathway was enriched in the tumor compartment of infiltrated and excluded tumors compared to desert tumors.

It is also worth noting that the findings in this study may inform new therapeutic strategies for cancer immunotherapies. For example, we found the two triggering receptors of myeloid cells, TREM1 and TREM2, differentially expressed in MDSC-like vs. TAM-like cells. High TREM1 expression in macrophages infiltrating human tumors has been shown to be associated with aggressive tumor behavior and poor patient survival. Ho, C. C. et al., Am J Respir Crit Care Med, 177:763-770 (2008). On the other hand, TREM2 has been shown to act as a tumor suppressor in hepatocellular carcinoma (Tang, W. et al., Oncogenesis, 8:9 (2019)) and colorectal cancer (Kim, S. M. et al., Cancers (Basel), 11 (2019). Targeting TREM molecules has recently drawn increased attention as a novel therapeutic opportunity for the treatment of inflammatory disorders and cancer (Nguyen et al., 2015). Our findings on the specific linkage of TREM1 and TREM2 to different subsets of myeloid cells associated with distinct tumor immune phenotypes, provides additional insights into their potential roles and may inform therapeutic strategies for targeting TREM molecules.

Lastly, we dissected how the cellular components of the tumor, stromal and immune compartments may interact through chemokine-receptor signaling, and thereby help to shape the tumor immunity continuum. Our study revealed a mechanism by which tumor cells may potentially mediate T cell recruitment via the CXCR6-CXCL16 axis. We found CXCL16 to be expressed by myeloid cells as previously described (van der Voort, R. et al., Arthritis Rheum, 52:1381-1391 (2005), but more importantly, our analysis revealed that CXCL16 is also expressed on tumor cells, especially in infiltrated and excluded ovarian tumor cells. CXCL16 is known to signal through the chemokine receptor CXCR6 (Wilbanks et al., 2001), and we found the highest expression of CXCR6 on CD4+ FOXP3 Treg cells and dysfunctional CD8+ GZMB T cells. These observations suggest potential recruitment of these T cell subsets by tumor cells in infiltrated and excluded tumors. Supporting these findings, it has been previously shown that ionizing radiation can induce the secretion of CXCL16, which would otherwise recruit CXCR6+CD8+ activated T cells to the tumor in a poorly immunogenic breast cancer mouse model. Matsumura, S. et al., J Immunol. 181:3099-3107 (2008). Hence, the CXCL16-CXCR6 axis could represent an important factor contributing to the tumor immunity continuum in ovarian cancer. Nevertheless, the effect of the CXCL16 chemotaxis gradient might be different between excluded and infiltrated tumors, whereby T cells cannot reach the tumor epithelium in excluded tumors despite the presence of a chemokine gradient. In fact, we found a large fraction of myofibroblasts in the excluded tumors not only express myofibroblast-specific marker ACTA2 (αSMA) (Sahai, E. et al., Nat Rev Cancer, 20:174-186 (2020)), but also collagen genes (fCOL10A1, COL11A1, COL6A3, COL1A1) and genes previously shown to contribute to reactive stroma (POSTN, MMP11, FN1) (Planche, A. et al., PLoS One, 6:e18640 (2011); Ryner, L et al., Clin Cancer Res, 21:2941-2951 (2015)). These observations further support the hypothesis that specific CAFs create a physical barrier that block the access of T cells to the tumor epithelium by producing a dense extracellular matrix. Salmon, H. et al., J Clin Invest, 122:899-910 (2012); Zeltz, C. et al., Semin Cancer Biol, 62:166-181 (2020).

Our study has several limitations worth noting. By sorting live cells based on their compartment of origin (i.e. immune, stromal or tumor compartment), we were able to enrich for low abundant cell populations and achieve a high-resolution dissection of each compartment. However, such an approach does not allow to describe the composition of the microenvironment as a whole and can only report the proportion of the identified cell states relative to their respective individual compartment. In addition, it has been reported that ovarian tumors and their immune microenvironments are heterogeneous. Jimenez-Sanchez, A. et al., Cell, 170:927-938 e920 (2017); Roberts, C. M. et al., Cancers (Basel): 11 (2019). Although our study has utilized different tumor sections from the same primary tumor and employed orthogonal methods (transcriptome classifier-based predictions and the CD8 IHC categorization) to assign a tumor immune phenotype for each tumor, it is still possible that such classification may not represent the whole tumor. Future studies including characterization of multiple segments of the same tumor as well as metastatic tumors of different sites from the same patient may provide additional insights into the heterogeneous tumor microenvironment of ovarian cancer. Finally, our chemokine receptor-ligand analysis across the different compartments is derived from a transcriptomic analysis only and further validation of these potential interactions by high-dimensional multiplex in situ analysis and functional assays in future studies are warranted.

To conclude, our study provides an in-depth dissection of the diverse cellular and functional phenotypes in the TME and their dynamic interplay, enabling a richer characterization of the tumor immunity continuum. Our work also provides additional insights into the biology that may help to shape the TME and immune phenotype. Finally, our findings may also enable identification of therapeutic targets and inform novel therapeutic strategies for overcoming immune suppression and increasing or expanding response to cancer immunotherapies.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of designing a treatment protocol for a human patient with ovarian cancer comprising:
a. determining the expression level of at least one of GZMK, TREM1, and TREM2 in a tumor sample from the patient; and
b. comparing the expression level to the expression level in a reference sample, wherein an increased level of GZMK expression compared to the reference sample is associated with reduced survival, an increased level of TREM1 compared to the reference sample is associated with the presence of MDSC-like myeloid cells, and an increased level of TREM2 compared to the reference sample is associated with the presence of TAM-like macrophages.

2. A method of treatment of ovarian cancer in a human patient comprising:
a. determining the expression level of at least one of GZMK, TREM1, and TREM2 in a tumor sample from the patient,
b. comparing the expression level to the expression level in a reference sample, wherein an increased level of GZMK expression is associated with reduced survival, an increased level of TREM1 is associated with the presence of MDSC-like myeloid cells, and an increased level of TREM2 is associated with the presence of TAM-like macrophages; and
c. if the patient has
i. increased expression of TREM1 compared to the reference sample, administering to the patient a therapy targeting MDSC-like myeloid cells;
ii. increased expression of TREM2 compared to the reference sample, administering to the patient a therapy targeting TAM-like macrophages; and/or
iii. decreased expression of GZMK compared to the reference sample, administering to the patient chemotherapy.

3. The method of treatment of claim 2, wherein after increased GZMK expression has been shown in the tumor:
a. chemotherapy is stopped, and/or
b. palliative care is given to the patient.

4. (canceled)

5. A method of characterizing an ovarian cancer in a human patient as a desert, excluded, or infiltrated type of tumor comprising:
a. determining the expression level of at least one of GZMB, GZMK, CD8A, TREM1, and TREM2 in a tumor sample from the patient,
b. comparing the expression level to the expression level in a reference sample, wherein higher expression of GZMB compared to the reference sample is associated with infiltrated type of tumors, higher expression of GZMK compared to the reference sample is associated with excluded tumors, higher expression of CD8A/GZMK double positive cells compared to the reference sample is associated with excluded tumors, higher expression of TREM1 compared to the reference sample is associated with desert tumors, and higher expression of TREM2 compared to the reference sample is associated with excluded and infiltrated tumors.

6. A method of treating a human patient with ovarian cancer comprising:
a. characterizing an ovarian cancer as desert, excluded, or infiltrated by determining the expression level of at least one of GZMB, GZMK, CD8A, TREM1, and TREM2 in a tumor sample from the patient,
b. comparing the expression level to the expression level in a reference sample, wherein higher expression of GZMB compared to the reference sample is associated with infiltrated type of tumors, higher expression of GZMK compared to the reference sample is associated with excluded tumors, higher expression of CD8A/GZMK double positive cells compared to the reference sample is associated with excluded tumors, higher expression of TREM1 compared to the reference sample is associated with desert tumors, and higher expression of TREM2 compared to the reference sample is associated with excluded and infiltrated tumors and
c. treating the patient with:
i. chemotherapy or autologous/allogenic effector cells if the higher expression of TREM1 suggests a desert tumor;
ii. immunotherapy (including checkpoint therapy) if the higher expression of GZMB and/or TREM2 suggests an infiltrated tumor;
iii. palliative care if the higher expression of GZMK suggests an excluded tumor;
iv. palliative care if the higher expression of CD8A/GZMK double positive cells suggests an excluded tumor; and/or
v. immune effector cells, such as T cell trafficking modulators, epigenetic modulators, tumor microenvironment (TME) remodeling molecules, and/or radiation therapy.

7. The method of claim 2, wherein the reference sample is data compiled across a plurality of patients and/or subjects.

8. The method of claim 2, where the reference sample is a healthy subject.

9. The method of claim 2, wherein the reference sample is a patient who responded to therapy.

10. The method of claim 2, wherein the reference sample is a patient who has a known desert tumor, a known excluded tumor, or a known infiltrated tumor.

11. (canceled)

12. (canceled)

13. (canceled)

14. The method of claim 2, wherein the method comprises evaluating all of GMZK, TREM1, and TREM2 in a tumor cell from the patient.

15. The method of claim 2, wherein the method further comprises obtaining a tumor sample from the patient before determining the expression level of at least one of GZMK, TREM1, and TREM2.

16. The method of claim 2, wherein the expression level of GZMK, TREM1, and/or TREM2 is determined using immunohistochemistry.

17. The method of claim 2, wherein the expression level of GZMK, TREM1, and/or TREM2 is determined by measuring mRNA transcript levels.

18. The method of claim 17, wherein the method further comprises determining the expression level of at least one reference gene in the tumor sample.

19. The method of claim 17 wherein the method further comprises normalizing the level of the mRNA transcripts against a level of an mRNA transcript of the at least one reference gene in the tumor sample to provide a normalized level of the mRNA transcript of GZMK, TREM1, and/or TREM2.

20. The method of claim 17, wherein the levels of the mRNA transcripts is determined by scRNAseq.

21. The method of claim 2, wherein the tumor sample is separated into tumor, stromal, and immune cells before evaluating the expression level of at least one of GZMK, TREM1, and or TREM2.

22. The method of claim 21, wherein the cell separation occurs through FACS.

23. The method of claim 19, wherein an increased normalized level of mRNA transcripts of GZMK is in CD8+ T cells.

24. The method of claim 23, wherein the number of CD8+ T cells that are GZMK positive are greater than the number of CD8+ T cells that are GZMK negative.

25. The method of claim 19, wherein the increased normalized level of mRNA transcripts of TREM2 is in macrophages.

26. The method of claim 2, wherein the chemotherapy comprises Albumin bound paclitaxel (nab-paclitaxel (Abraxane®)), altretamine (Hexalen®), Capecitabine (Xeloda®), carboplatin, cisplatin, cyclophosphamide (Cytoxan®), docetaxel (Taxotere®), etoposide (VP-16), gemcitabine (Gemzar®), ifosfamide (Ifex®), irinotecan (CPT-11 (Camptosar®)), doxorubicin (such as liposomal doxorubicin (Doxil®)), melphalan, paclitaxel (Taxol®), pemetrexed (Alimta®), topotecan, vinorelbine (Navelbine®), Niclosamide, Metformin, BAY 87-2243, Decitabine, Guadecitabine, Azacytidine, Abagovomab, Oregovomab, NeoVax with Nivolumab, Anlotinib, Enoxaparin with Rosuvastatin, Niraparib, Chiauranib, Trabectedin with pegylated liposomal Doxorubicin, ACB-S6-500, SGI-110, Letrozole, Pazopanib, Palbociclib, Apatinib, Masitinib, Cabazitaxel, IMAB027, Fludarabine, ABT-888, Fostamatinib, Olaparib, Temozolomide, Talazoparib, P53-SLP, OMP-54F28, Hydralazine and magnesium valproate, Fludarabine, Lapatinib, Bendamustine HCL, Sorafenib, Camrelizumab, Tremelilumab, Tocotrienol, and/or Exemestane.

27. The method of claim 2, wherein a therapy targeting MDSC myeloid cells comprises:
a. Cisplatin, 5-flurouracil, gemcitabine, and/or paclitaxel;
b. Liver X receptor (LXR) beta agonist;
c. a checkpoint inhibitor, such as
i. anti-PD-1 therapy (such as anti-PD-1 antibodies including pembrolizumab (Ketruda®), nivolumab (Opdivo®), cemiplimab (Libtayo®), Toripalimab, CT-011, monoclonal antibody HX0088, and antibody AK105;
ii. anti-PD-L1 therapy (such as anti PD-L1 antibodies including atezolizumab (Tecentriq®), avelumab (Bavencio®), durvalumab (MEDI4736 anti-PD-L1; Imfinzi®), Tremelimumab, mAb ZKAB001, Tremelimumab, and Ramucirumab (Cyramza); and/or
d. Anti-TGFβ therapy (such as anti-TGFβ antibodies including Pembrolizumab and Fresolimumab).

28. (canceled)
